(19) United States
(12) Patent Application Publication
(10) Pub. No.: US 2004/0009164 A1
(43) Pub. Date: Jan. 15, 2004

(54) PSP94 diagnostic reagents and assays

(76) Inventors: Jonathan Reeves, Hawkesbury (CA); Jerome Edward Tanner, Dollard-des-Ormeaux (CA); Chandra J. Panchal, London (CA)

Correspondence Address: Ronald S. Kosie, BROUILLETTE KOSIE PRINCE, 25th Floor, 1100 Rene-Levesque Boulevard West, Montreal, QC, H3B 5C9, CA

(21) Appl. No.: 10/429,668

(22) Filed: May 1, 2003

(30) Foreign Application Priority Data
May 1, 2002 (CA) 2,380,662
Jun. 25, 2002 (CA) 2,391,438

(51) Int. Cl. C12Q 1/68; C07H 21/04; A61K 38/46; C12N 9/20; C12P 21/02; C12N 5/06

(52) U.S. Cl. 424/94.6; 435/6; 435/69.1; 435/198; 435/320.1; 435/325; 536/23.2

(57) ABSTRACT

In the serum, PSP94 occurs as a free form or is associated with a carrier protein. PSP94 in its bound form has been quantified in the blood of prostate cancer patients and these measurements have shown utility as evaluation of prognosis. The present invention identifies a carrier protein to which PSP94 is bound (named PSP94-binding protein) its purification process, its nucleic acid and amino acid sequence and to the use of these sequences in the diagnosis and prognosis of PSP94 related disease. More particularly, the present invention discloses improved diagnostic and prognostic assays as well as reagents useful for the evaluation of conditions linked with abnormal or elevated levels of PSP94, such as prostate cancer and benign prostatic hyperplasia.

### FIELD OF THE INVENTION

This invention relates to new polypeptides able to bind PSP94 (PSP94-binding protein), as well as nucleic acid and amino acid sequences, and the use of these sequences in the diagnosis and prognosis of diseases.

This invention also relates to improved diagnostic assays, kit and reagents such as antibodies able to recognize PSP94 or a PSP94-binding protein.

### BACKGROUND OF THE INVENTION

The prostate gland, which is found exclusively in male mammals, produces several components of semen and blood and several regulatory peptides. The prostate gland comprises stromal and epithelial cells, the latter group consisting of columnar secretory cells and basal nonsecretory cells. A proliferation of these basal cells as well as stromal cells gives rise to benign prostatic hyperplasia (BPH), which is one common prostate disease. Another common prostate disease is prostatic adenocarcinoma (CaP), which is the most common of the fatal pathophysiological prostate cancers, and involves a malignant transformation of epithelial cells in the peripheral region of the prostate gland. Prostatic adenocarcinoma and benign prostatic hyperplasia are two common prostate diseases, which have a high rate of incidence in the aging human male population.

Approximately one out of every four males above the age of 55 suffers from a prostate disease of some form or another. Prostate cancer is the second most common cause of cancer related death in elderly men, with approximately 185,000 cases diagnosed and about 39,000 deaths reported annually in the United States.

Studies of the various substances synthesized and secreted by normal, benign and cancerous prostates carried out in order to gain an understanding of the pathogenesis of the various prostate diseases reveal that certain of these substances may be used as immunohistochemical tumor markers in the diagnosis of prostate disease. The three predominant proteins or polypeptides secreted by a normal prostate gland are: (1) Prostatic Acid Phosphatase (PAP); (2) Prostate Specific Antigen (PSA); and, (3) Prostate Secretory Protein of 94 amino acids (PSP94), which is also known as Prostatic Inhibin Peptide (PIP), Human Seminal Plasma Inhibin (HSPI), or β-microseminoprotein (β-MSP), and which is hereinafter referred to as PSP94.

PSP94 is a simple non-glycosylated cysteine-rich protein, and constitutes one of three predominant proteins found in human seminal fluid along with Prostate Specific Antigen (PSA) and Prostate Acid Phosphatase (PAP). PSP94 has a molecular weight of 10.7 kDa, and the complete amino acid sequence of this protein has already been determined. The cDNA and gene for PSP94 have been cloned and characterized (Ulvsback, et al., Biochem. Biophys. Res. Comm., 164:1310, 1989; Green, et al., Biochem. Biophys. Res. Comm., 167:1184, 1990). Immunochemical and in situ hybridization techniques have shown that PSP94 is located predominantly in prostate epithelial cells. It is also present, however, in a variety of other secretory epithelial cells (Weiber, et al., Am. J. Pathol., 137:593, 1990). PSP94 has been shown to be expressed in prostate adenocarcinoma cell line, LNCap (Yang, et al., J. Urol., 160:2240, 1998). As well, an inhibitory effect of exogenous PSP94 on tumor cell growth has been observed both in vivo and in vitro (Garde, et al., Prostate, 22:225, 1993; Lokeshwar, et al., Cancer Res., 53:4855, 1993), suggesting that PSP94 could be a negative regulator for prostate carcinoma growth via interaction with cognate receptors on tumor cells.

Native PSP94 has been shown to have a therapeutic effect in the treatment of hormone refractory prostate cancer (and potentially other prostate indications). For example, PSP94 expression within prostate cancer is known to decrease as tumor grade and agressivity increases. Tumor PSP94 expression is stimulated upon anti-androgen treatment, particularly in high grade tumors. U.S. Pat. No. 5,428,011 (Sheth A. R. et al., issued 1995-06-27), incorporated herein by reference, describes pharmaceutical preparations comprising native PSP94 used in the in-vitro and in-vivo inhibition of prostate, gastrointestinal and breast tumor growth. These pharmaceutical preparations include either native PSP94 alone or a mixture of native PSP94 and an anticancer drug such as, for example, mitomycin, idalubicin, cisplatin, 5-fluorouracil, methotrexate, adriamycin and daunomycin. In addition, the therapeutic effect of recombinant human PSP94 (rhuPSP94) and polypeptide analogues such as PCK3145 has been described in Canadian Patent Application No. 2,359,650 (incorporated herein by reference).

Immunohistochemical studies and investigations at the level of mRNA have shown that the prostate is a major source of PSP94. PSP94 is involved in the feedback control of, and acts to suppress secretion of, circulating follicle-stimulating hormone (FSH) both in-vitro and in-vivo in adult male rats. PSP94 acts both at the pituitary as well as at the prostate site since both are provided with receptor sites for PSP94. PSP94 has been demonstrated to suppress the biosynthesis and release of FSH from the rat pituitary as well as to possibly affect the synthesis/secretion of an FSH-like peptide by the prostate. These findings suggest that the effects of PSP94 on tumor growth in vivo, could be attributed to the reduction in serum FSH levels.

Recently, it has been shown that PSP94 concentrations in serum of patients with BPH or CaP are significantly higher than normal. The highest serum concentration of PSP94 observed in normal men is approximately 40 ng/ml, while in men with either BPH or CaP, serum concentrations of PSP94 have been observed up to 400 ng/ml.

In the serum, PSP94 occurs as a free (unbound) form or bound form associated with a carrier protein(s) of unknown identity. PSP94 in its bound form (state) has been quantified in the blood of prostate cancer patients and these measurements have been analyzed for their utility as prognostic evaluation (Bauman, G. S., et al., The Prostate J. 2:94-101, 2000; Xuan, J. W. U.S. Pat. No. 6,107,103; Wu, D. et al., J. Cell. Biochem. 76:71-83, 1999). It was suggested that measurements of the free and bound forms of PSP94 are likely to have a greater clinical relevance in several areas of prostate cancer than measurements of the free form alone. In addition, it was demonstrated that measurements of both forms of PSP94 allows an accurate prediction of relapse free interval in post-radiotherapy prostate cancer. However current assay for PSP94 measurement, such as the one described in U.S. Pat. No. 6,107,103 rely on a purification step for separating bound and free forms of the protein and therefore lack the simplicity necessary for a useful and efficient commercial assay.

### SUMMARY OF THE INVENTION

Methods for evaluating (quantifying) levels of PSP94 (free or bound forms of PSP94 as well as total PSP94) are described herein. The present invention relates to antibodies having specificity for PSP94 or a PSP94-binding protein and improved diagnostic and prognostic assays, hybridomas, kits and reagents thereof.

In addition, the carrier protein(s) to which PSP94 is bound is described, identified and characterized in the present application.

Due to its ability to be associated with PSP94, a PSP94-binding protein(s) and related antibodies may have an impact on the biological activity of PSP94 and may therefore be used herein as a diagnostic and prognostic marker of (PSP94-related) disease.

This invention therefore relates to polypeptides (SEQ ID NO.:2, SEQ ID NO.:3, SEQ ID NO.:7, SEQ ID NO.:8, SEQ ID NO.:9) identified herein as PSP94-binding protein(s), purification process, nucleic acid and amino acid sequence and the use of these sequences in the diagnosis, and prognosis of diseases (e.g., prostate cancer or diseases characterized by abnormal or elevated levels of PSP94 and/or follicle stimulating hormone (FSH) and/or abnormal or elevated levels of a PSP94-binding protein).

In a first aspect, the present invention provides a (e.g., isolated) polynucleotide (e.g., encoding a PSP94-binding protein), which may comprise a member selected from the group consisting of

a) a polynucleotide as set forth in SEQ ID NO.: 1,

b) a polynucleotide as set forth in SEQ ID NO.: 6,

c) a polynucleotide having sequence 1 to 1392 of SEQ ID NO.:6,

d) a polynucleotide having sequence 1 to 1653 of SEQ ID NO.:6,

e) a polynucleotide of a size between 10 and 2005 (or 2004) bases in length identical in sequence to a contiguous portion of at least 10 bases of the polynucleotide as set forth in SEQ ID NO.: 1, and

f) a polynucleotide of a size between 10 and 1876 (or 1875) bases in length identical in sequence to a contiguous portion of at least 10 bases of the polynucleotide as set forth in SEQ ID NO.: 6.

The polynucleotide may preferably be the polynucleotide as set forth in SEQ ID NO.:1 or the polynucleotide as set forth in SEQ ID NO.:6 or the polynucleotide having sequence 1 to 1392 of SEQ ID NO.:6 or a polynucleotide having sequence 1 to 1653 of SEQ ID NO.:6. The polynucleotide of the present invention may particularly be chosen based on the ability of the encoded protein to bind PSP94. It is to be understood herein that SEQ ID NO.: 1 may be considered an analogue of SEQ ID NO.: 6.

In a second aspect, the present invention provides polypeptides and polypeptides analogues such as for example,

a polypeptide as set forth in SEQ ID NO.: 2,

a polypeptide as set forth in SEQ ID NO.: 3,

a polypeptide as set forth in SEQ ID NO.: 7,

a polypeptide as set forth in SEQ ID NO.: 8,

a polypeptide as set forth in SEQ ID NO.: 9,

a polypeptide of a size between 10 and 505 amino acids in length identical to a contiguous portion of the same size of SEQ ID NO.:2,

a polypeptide of a size between 10 and 592 amino acids in length identical to a contiguous portion of the same size of SEQ ID NO.:3,

a polypeptide of a size between 10 and 624 amino acids in length identical to a contiguous portion of the same size of SEQ ID NO.:7,

a polypeptide analogue having at least 90% of its amino acid sequence identical to the amino acid sequence set forth in SEQ ID NO: 2, in SEQ ID NO.:3, in SEQ ID NO.:7, in SEQ ID NO: 8 or in SEQ ID NO.:9,

a polypeptide analog having at least 70% of its amino acid sequence identical to the amino acid sequence set forth in SEQ ID NO: 2, in SEQ ID NO.:3, in SEQ ID NO.:7, in SEQ ID NO: 8 or in SEQ ID NO.:9,

a polypeptide analog having at least 50% of its amino acid sequence identical to the amino acid sequence set forth in SEQ ID NO: 2 in SEQ ID NO.:3, in SEQ ID NO.:7, in SEQ ID NO: 8 or in SEQ ID NO.:9,

a polypeptide analogue having at least 90% of its amino acid sequence identical to the amino acid sequence of

a polypeptide of a length from between 10 and 505 contiguous amino acids of SEQ ID NO.:2,

a polypeptide of a length from between 10 and 592 contiguous amino acids of SEQ ID NO.:3 or,

a polypeptide of a length from between 10 and 624 contiguous amino acids of SEQ ID NO.:7,

a polypeptide analogue having at least 70% of its amino acid sequence identical to the amino acid sequence of

a polypeptide of a length from between 10 and 505 contiguous amino acids of SEQ ID NO.:2,

a polypeptide of a length from between 10 and 592 contiguous amino acids of SEQ ID NO.:3 or,

a polypeptide of a length from between 10 and 624 contiguous amino acids of SEQ ID NO.:7,

a polypeptide analogue having at least 50% of its amino acid sequence identical to the amino acid sequence of

a polypeptide of a length from between 10 and 505 contiguous amino acids of SEQ ID NO.:2,

a polypeptide of a length from between 10 and 592 contiguous amino acids of SEQ ID NO.:3 or,

a polypeptide of a length from between 10 and 624 contiguous amino acids of SEQ ID NO.:7.

In accordance with the present invention, the polypeptide may preferably be the polypeptide as set forth SEQ ID NO.: 2, the polypeptide as set forth SEQ ID NO.: 3, the polypeptide as set forth SEQ ID NO.:7, the polypeptide as set forth SEQ ID NO.:8 or the polypeptide as set forth SEQ ID NO.:9. The polypeptide of the present invention may particularly be chosen based on its ability to bind PSP94. It is to be understood herein that SEQ ID NO.: 2 and SEQ ID NO.: 3 may be considered analogues of SEQ ID NO.: 7. SEQ ID NO.: 8 and SEQ ID NO.:9 may also be considered analogues of SEQ ID NO.:7.

In an additional aspect, the present invention provides an immunizing composition including, for example, a vector comprising a polynucleotide as defined herein. It is sometimes preferable to have a polynucleotide of at least 21 bases in length of a desired sequence since a polypeptide of 7 amino acids (encoded by a 21 base pair polynucleotide sequence) is often associated with the major histocompatibility complex (MHC) during antigen presentation. The vector may comprise, for example, a polynucleotide selected from the group consisting of a polynucleotide as set forth in SEQ ID NO.: 1, a polynucleotide as set forth in SEQ ID NO.: 6, a polynucleotide having sequence 1 to 1392 of SEQ ID NO.:6, a polynucleotide having sequence 1 to 1653 of SEQ ID NO.:6, a polynucleotide of a size between 21 and 2005 bases in length identical in sequence to a contiguous portion of the same size of the polynucleotide set forth in SEQ ID NO.: 1 or a polynucleotide of a size between 21 and 1876, bases in length, identical in sequence to a contiguous portion of the same size of the polynucleotide set forth in SEQ ID NO.: 6, and a diluent or buffer. It is to be understood herein that the vector may enable the expression of a polypeptide encoded from said polynucleotide. The vector may be linear or circular and may contain minimal sequences in addition to the polynucleotide itself (e.g., sequence for integration into the genome, promoter, CpG sequences). Administration of a polynucleotide of the present invention (without any additional sequence, i.e, without a vector) may sometimes be sufficient to initiate a desired immune response.

In a further aspect, the present invention relates to an immunizing composition comprising a polypeptide as defined herein (e.g., SEQ ID NO.:2, SEQ ID NO.:3, SEQ ID NO.:7, SEQ ID NO.:8, SEQ ID NO.:9), a polypeptide analogue, variant, fragment or combination thereof and a diluent or a buffer. Immunization with a combination of any of the immunizing composition described herein is also encompassed by the present invention.

The immunizing composition(s) may further comprise an adjuvant. In an additional embodiment, the immunizing composition may also comprise PSP94 (native and/or recombinant), PSP94 variant, PSP94 fragment, a vector comprising a polynucleotide encoding PSP94, a polynucleotide encoding a PSP94 variant, a polynucleotide encoding a PSP94 fragment and combination thereof. Again, the vector may enable the expression of a polypeptide encoded from said polynucleotide. For reference on native PSP94, recombinant PSP94 (e.g., rHuPSP94), PSP94 variants, analogues and fragments, please see Canadian patent application No.: 2,359,650 or international patent application, published under No. WO 02/33090.

In a further aspect, the present invention relates to a method of (for) generating an antibody (monoclonal or polyclonal) to a polypeptide (e.g., PSP94, PSP94-binding protein and/or PSP94/PSP94-binbing protein complex), said method comprising administering to a mammal an immunizing composition (comprising a polypeptide, polypeptide analogue, a polynucleotide and combination thereof etc.) as defined herein.

In accordance with the present invention, mammals that may be immunized using the present method include, for example, a human, a mouse, a rabbit, a sheep, a horse, a cow, a rat, a pig, and other mammals having a functional immune system. A “mammal having a functional immune system” is to be understood herein as a mammal able to produce antibodies (immunoglobulins) when immunized with an antigen (i.e., having a humoral immune response and/or a cellular immune response to the antigen).

Further aspects of the present invention relate to a monoclonal antibody produced by the hybridoma cell line deposited to the ATCC under Patent Deposit No.: PTA-4242 and antigen binding fragments thereof, to a monoclonal antibody produced by the hybridoma cell line deposited to the ATCC under Patent Deposit No.: PTA-4243 and antigen binding fragments thereof, to an hybridoma cell line deposited to the ATCC under Patent Deposit No.: PTA-4242 and to a hybridoma cell line deposited to the ATCC under Patent Deposit No.: PTA-4243.

In an additional aspect, the present invention relates to a cell that has incorporated (has been transformed, transduced, transfected, etc.) with any of the polynucleotide of the present invention e.g., SEQ ID NO.: 1, SEQ ID NO.:6, antisenses, fragments, variants, mRNA, etc.

In yet an additional aspect, the present invention relates to a (isolated) cell that has incorporated and/or that is expressing at least one of the polypeptides of the present invention, e.g., SEQ ID NO.: 2, SEQ ID NO.:3, SEQ ID NO.:7, SEQ ID NO.:8, SEQ ID NO.:9, variants, fragments, analogues or combination thereof.

In another aspect, the present invention comprises the use of a polynucleotide as defined herein (SEQ ID NO.:1, SEQ ID NO.:6, fragments, antisense, analogues, mRNA), in the diagnosis or prognosis, (or treatment) of a condition linked with abnormal (e.g., high, elevated) levels of PSP94, or with abnormal (e.g., high, elevated) levels of a PSP94-binding protein.

In yet another aspect, the present invention provides the use of the polypeptide as defined herein (e.g., SEQ ID NO.:2, SEQ ID NO.:3, SEQ ID NO.:7, SEQ ID NO.:8, SEQ ID NO.:9, analogue, variant, fragments) in the diagnosis or prognosis, (or treatment) of a condition linked with abnormal (e.g., high, elevated) levels of PSP94 or with abnormal (e.g., high, elevated) levels of a PSP94-binding protein.

In accordance with the present invention the polynucleotide defined herein or the polypeptide defined herein may be used in the diagnosis, or prognosis of a condition such as, for example, prostate cancer, stomach cancer, breast cancer, endometrial cancer, ovarian cancer, other cancers of epithelial secretion and benign prostate hyperplasia (BPH) or a disease characterized with an elevated level of FSH.

In an additional aspect, the present invention relates to a method for measuring, in a sample, the amount of a polypeptide as defined herein, for example, a polypeptide selected from the group consisting of SEQ ID NO.: 2, SEQ ID NO.: 3, SEQ ID NO.:7, SEQ ID NO.:8 and SEQ ID NO.:9 (as well variants, analogues and fragments thereof) or combination thereof. In accordance with the present invention, the method may comprise contacting said sample with a molecule (an antibody or a polypeptide) able to recognize said polypeptide. The method contemplated herein may be applied to polypeptides that are immobilized to a blot membrane, a plate, a matrix or not (in solution).

It is to be understood herein that in order to develop a quantitative assay to assess the level of a polypeptide, a preferred molecule may have sufficient affinity and specificity for the desired polypeptide. Affinity and specificity may be determined, for example, by comparing binding of the molecule to irrelevant polypeptides, by competition assays for the polypeptide of interest, etc.

In one embodiment of the present invention, the molecule used for the above described method may include, for example, the monoclonal antibody produced by the hybridoma cell line deposited to the ATCC under Patent Deposit No.: PTA-4242 and the monoclonal antibody produced by the hybridoma cell line deposited to the ATCC under Patent Deposit No.: PTA-4243. In another embodiment of the present invention, the molecule may be, for example PSP94 and analogues thereof.

The method for measuring the amount of a polypeptide selected from the group consisting of SEQ ID NO.: 2, SEQ ID NO.: 3, SEQ ID NO.:7, SEQ ID NO.:8 and SEQ ID NO.:9 contemplated herein may further comprise, for example, the following steps:

a) bringing a sample comprising at least one of the polypeptide of the present invention into contact with an antibody immobilized to a suitable substrate (e.g., ELISA plate, matrix, SDS-PAGE, Western blot membranes),

b) adding to step a) a detection reagent comprising a label or marker, and;

c) detecting a signal resulting from a label or marker.

Suitable detection reagents may comprise, for example, an antibody or a polypeptide having an affinity for a polypeptide(s) of the present invention, and the detection reagent may have preferably, a different binding site than the antibody. As described herein, the detection reagent may either be directly coupled (conjugated) to a label (or marker) or able to be recognized by a second molecule carrying (conjugated with) said label or marker.

An example of an antibody that may be used in step a) is the monoclonal antibody (17G9) produced by the hybridoma cell line deposited to the ATCC under Patent Deposit No.: PTA-4243. In that case, the monoclonal antibody (3F4) produced by the hybridoma cell line deposited to the ATCC under Patent Deposit no.: PTA-4242 may be used as a detection reagent in step c).

Any antibodies able to bind to a PSP94-binding protein (SEQ ID NO.:2, SEQ ID NO.:3, etc.), such as those antibodies listed in table 10 (identified as clones), may be used in the methods described herein (e.g., (clone) 2B10, 1B11, 9B6, P8C2, B3D1, 26B10). When two antibodies are needed to perform the present methods it may be preferable to choose antibodies binding to different epitopes.

Another example of an antibody that may be used in step a) is the monoclonal antibody (3F4) produced by the hybridoma cell line deposited to the ATCC under Patent Deposit no.: PTA-4242. In that case the monoclonal antibody (17G9) produced by the hybridoma cell line deposited to the ATCC under Patent Deposit no.: PTA-4243 may be used as a detection reagent in step c).

In a further aspect, the present invention relates to a method for measuring, in a sample the amount of a polypeptide selected from the group consisting of SEQ ID NO.: 2, SEQ ID NO.:3, SEQ ID NO.:7, SEQ ID NO.:8 and SEQ ID NO.:9 (variants, analogues, fragments) or combination thereof, that is not bound (i.e., free (unbound)) to PSP94, said method comprising;

a) removing, from said sample, a complex formed by PSP94 and any one of the polypeptide selected from the group consisting of SEQ ID NO.: 2, SEQ ID NO.:3, SEQ ID NO.:7, SEQ ID NO.:8 and SEQ ID NO.:9 (variants, analogues, fragments) generating a complex-free sample, and;

b) contacting said complex-free sample with an antibody able to recognize any one of the polypeptide selected from the group consisting of SEQ ID NO.: 2, SEQ ID NO.:3, SEQ ID NO.:7, SEQ ID NO.:8 and SEQ ID NO.:9 (variants, analogues, fragments) and combination thereof.

In one embodiment of the present invention, the antibody used in step b) may be selected from the group consisting of the monoclonal antibody produced by the hybridoma cell line deposited to the ATCC under Patent Deposit No.: PTA-4242 and the monoclonal antibody produced by the hybridoma cell line deposited to the ATCC under Patent Deposit No.: PTA-4243.

The method for measuring the amount of the polypeptide of the present invention that is not bound to PSP94 contemplated above may, for example, comprise the following step;

a) removing, from said sample, a complex formed by PSP94 and any one of the polypeptide selected from the group consisting of SEQ ID NO.: 2, SEQ ID NO.:3, SEQ ID NO.:7, SEQ ID NO.:8 and SEQ ID NO.:9, generating a complex-free,

b) immobilizing (coating, adsorbing) an antibody to a suitable substrate (ELISA plate, matrix, SDS-PAGE, Western blot membranes),

c) adding said complex-free sample,

d) adding a detection reagent comprising a label or marker, and;

e) detecting a signal resulting from a label or marker.

The removal of the complex may be performed, for example, by using the monoclonal antibody produced by the hybridoma cell line deposited to the ATCC under Patent Deposit No.: PTA-4241.

Suitable antibodies that may be used in step b) are antibodies selected from the group consisting of the monoclonal antibody (3F4) produced by the hybridoma cell line deposited to the ATCC under Patent Deposit No.: PTA-4242 and the monoclonal antibody (17G9) produced by the hybridoma cell line deposited to the ATCC under Patent Deposit No.: PTA-4243.

In an additional aspect, the present invention includes the use of an (monoclonal) antibody selected from the group consisting of a monoclonal antibody (2D3) produced by the hybridoma cell line deposited to the ATCC under Patent Deposit No.: PTA-4240, a monoclonal antibody (P1E8) produced by the hybridoma cell line deposited to the ATCC under Patent Deposit No.: PTA-4241, a monoclonal antibody (3F4) produced by the hybridoma cell line deposited to the ATCC under Patent Deposit No.: PTA-4242 and a monoclonal antibody (17G9) produced by the hybridoma cell line deposited to the ATCC under Patent Deposit No.: PTA-4243, for evaluating (in a sample) the amount (quantity, concentrations) (free, bound, and/or total amounts) of SEQ ID NO.:2, SEQ ID NO.: 3, SEQ ID NO.:7, SEQ ID NO.:8, SEQ ID NO.:9, variants, fragments, analogues, and/or combination thereof.

In another aspect, the present invention includes the use of a molecule selected from the group consisting of a polypeptide as set forth in SEQ ID NO.:2, a polypeptide as set forth in SEQ ID NO.: 3, a polypeptide as set forth in SEQ ID NO.: 7, a polypeptide as set forth in SEQ ID NO.: 8, a polypeptide as set forth in SEQ ID NO.: 9, a monoclonal antibody (2D3) produced by the hybridoma cell line deposited to the ATCC under Patent Deposit No.: PTA-4240, a monoclonal antibody (P1E8) produced by the hybridoma cell line deposited to the ATCC under Patent Deposit No.: PTA-4241, a monoclonal antibody (3F4) produced by the hybridoma cell line deposited to the ATCC under Patent Deposit No.: PTA-4242 and a monoclonal antibody (17G9) produced by the hybridoma cell line deposited to the ATCC under Patent Deposit No.: PTA-4243, for evaluating (in a sample) the amount of PSP94 or for the diagnostic of a condition linked with abnormal or elevated levels of PSP94 or of a PSP94-binding protein.

In another aspect, the present invention relates to an antibody conjugate comprising a first moiety and a second moiety, said first moiety being selected from the group consisting of a monoclonal antibody (2D3) produced by the hybridoma cell line deposited to the ATCC under Patent Deposit No.: PTA-4240, a monoclonal antibody (P1E8) produced by the hybridoma cell line deposited to the ATCC under Patent Deposit No.: PTA-4241, a monoclonal antibody (3F4) produced by the hybridoma cell line deposited to the ATCC under Patent Deposit No.: PTA-4242 and a monoclonal antibody (17G9) produced by the hybridoma cell line deposited to the ATCC under Patent Deposit No.: PTA-4243 and said second moiety being selected from the group consisting of a pharmaceutical agent, a solid support, a reporter molecule, a group carrying a reporter molecule, a chelating agent, an acylating agent, a cross-linking agent, and a targeting group, wherein said second moiety or conjugation of said second moiety does not interfere with the biological activity (e.g., affinity, stability) of the first moiety.

In one embodiment of the present invention, examples of solid support may consist in carbohydrates, liposomes, lipids, colloidal gold, microparticles, microcapsules, microemulsions, and the matrix of an affinity column.

In an additional embodiment, reporter molecule may be selected from the group consisting of a fluorophore (e.g., rhodamine, fluoroscein, and green fluorescent protein), a chromophore, a dye, an enzyme (e.g., alkaline phosphatase, horseradish peroxidase, beta-galactosidase, chloramphenicol acetyl transferase), a radioactive molecule and a molecule of a binding/ligand (e.g., biotin/avidin (streptavidin)) complex.

In yet an additional embodiment, the pharmaceutical agent may be selected from the group of a toxin (e.g., bacterial toxins), a (e.g., anti-cancer) drug and a pro-drug.

In a further aspect, the present invention includes a kit for use in evaluating (in a sample) the amount of PSP94 or for the diagnosis of a condition linked with abnormal (e.g., high, elevated) levels of PSP94 (or of a PSP94-binding protein) comprising a container having a molecule able to recognize (bind) PSP94. It is to be understood herein that the kit may be provided (sold) in separate constituents.

In one embodiment of the present invention, the molecule able to recognize PSP94 that may be included in the kit, may (comprise, for example) be a molecule selected from the group consisting of (one or more of the following) a monoclonal antibody (2D3) produced by the hybridoma cell line deposited to the ATCC under Patent Deposit No.: PTA-4240, a monoclonal antibody (P1E8) produced by the hybridoma cell line deposited to the ATCC under Patent Deposit No.: PTA-4241, a monoclonal antibody (3F4) produced by the hybridoma cell line deposited to the ATCC under Patent Deposit No.: PTA-4242, a monoclonal antibody (17G9) produced by the hybridoma cell line deposited to the ATCC under Patent Deposit No.: PTA-4243, the antibody conjugate(s) of the present inventions and a polypeptide selected from the group consisting of SEQ ID NO.:2, SEQ ID NO.:3, SEQ ID NO.:7, SEQ ID NO.:8 and SEQ ID NO.:9.

In another embodiment of the present invention, the kit may further comprise a container having an antibody able to recognize (bind) a polypeptide selected from the group consisting of the polypeptide set forth in SEQ ID NO.:2, the polypeptide set forth in SEQ ID NO.:3 and the-polypeptide set forth in SEQ ID NO.:7, the polypeptide set forth in SEQ ID NO.:8, the polypeptide set forth in SEQ ID NO.:8, variant, fragment, analogues and combination thereof. Contemplated by the present invention are the monoclonal antibody (17G9) produced by the hybridoma cell line deposited to the ATCC under Patent Deposit No.: PTA-4243 and a monoclonal antibody (3F4) produced by the hybridoma cell line deposited to the ATCC under Patent Deposit No.: PTA-4242.

It is to be understood herein that kits may be provided in separate constituents. The antibodies provided with the kit may be in different forms such as bound to plates or membranes or other type of solid matrix or in vials containing concentrated forms or suitable working dilutions of the antibodies.

In another aspect, the present invention provides a method for preparing a polypeptide as defined herein (a PSP94-binding protein, e.g., a polypeptide selected from the group consisting of the polypeptide set forth in SEQ ID NO.:2, the polypeptide set forth in SEQ ID NO.:3, the polypeptide set forth in SEQ ID NO.:7, the polypeptide set forth in SEQ ID NO.:8 and the polypeptide set forth in SEQ ID NO.:9) comprising:

a) cultivating a host cell under conditions which provide for the expression of said polypeptide by the cell; and

b) recovering the polypeptide by one or more purification step.

In yet another aspect, the present invention provides a method for preparing the polypeptide as defined herein (a PSP94-binding protein, e.g., a polypeptide selected from the group consisting of the polypeptide set forth in SEQ ID NO.:2, the polypeptide set forth in SEQ ID NO.:3, the polypeptide set forth in SEQ ID NO.:7 the polypeptide set forth in SEQ ID NO.:8, the polypeptide set forth in SEQ ID NO.:9 and combination thereof) comprising:

a) collecting one or more biological sample containing said polypeptide; and

b) recovering the polypeptide by one or more purification step.

It is to be understood herein that the purification step either alone or in combination may be selected from the group consisting of ammonium sulfate precipitation, size exclusion chromatography, affinity chromatography, ion-exchange chromatography or the like.

In another embodiment of the present invention, the purification step may comprise;

a) adding ammonium sulfate to said biological sample,

b) performing ion-exchange chromatography,

c) performing affinity-chromatography using a PSP94-conjugated affinity matrix,

d) performing size-exclusion chromatography, and

e) recovering a fraction containing a substantially pure PSP94-binding protein.

In a further aspect, the present invention also includes a process for the purification of a PSP94-binding protein from a sample comprising:

a) adding ammonium sulfate to said sample (e.g., human male serum) in a manner as to provide precipitation of a PSP94-binding protein,

b) centrifuging the mixture of step a) to recover precipitated proteins,

c) resuspending said precipitated proteins,

d) performing ion-exchange chromatography to recover a fraction of proteins containing a PSP94-binding protein,

e) performing affinity-chromatography using a PSP94-conjugated affinity matrix to recover a fraction of proteins containing a PSP94-binding protein,

f) performing size exclusion chromatography to recover a fraction of proteins containing a PSP94-binding protein and;

g) recovering a fraction containing a substantially pure PSP94-binding protein (e.g., a polypeptide selected from the group consisting of the polypeptide defined in SEQ ID NO.:2, the polypeptide defined in SEQ ID NO.:3, the polypeptide defined in SEQ ID NO.:7, the polypeptide set forth in SEQ ID NO.:8, the polypeptide set forth in SEQ ID NO.:9 and combination thereof).

In one embodiment of the present invention, the precipitation of a PSP94-binding protein in step a) may be effected by adding ammonium sulfate to a final concentration of up to 47%.

In a second embodiment of the present invention, the ion-exchange chromatography of step d) may be performed by using an anion-exchange chromatography matrix.

The present invention in a further aspect thereof comprises a purification process for a PSP94-binding protein (e.g., a polypeptide selected from the group consisting of the polypeptide defined in SEQ ID NO.:2, the polypeptide defined in SEQ ID NO.:3, the polypeptide defined in SEQ ID NO.:7, the polypeptide defined in SEQ ID NO.:8, the polypeptide defined in SEQ ID NO.:9 and combination thereof) (summarized in FIG. 8). The purification of a PSP94-binding protein from serum may comprise, for example, the following steps:

a) adding ammonium sulfate to a human (male) serum sample to provide a solution with a final concentration of ammonium sulfate of 32%,

b) centrifuging the solution of the previous step to recover a pellet fraction of proteins containing unspecific human serum proteins and a supernatant fraction of proteins containing a PSP94-binding protein,

c) recovering the supernatant fraction of proteins containing a PSP94-binding protein and adjusting the concentration of ammonium sulfate to a final concentration of 47% to provide a solution of precipitated proteins containing a PSP94-binding protein,

d) centrifuging the mixture to recover precipitated proteins containing a PSP94-binding protein,

e) resuspending said precipitated proteins containing a PSP94-binding protein in an aqueous media (e.g., water, phosphate buffered saline, 10 mM MES, 10 mM MOPS, 10 mM Bicine: these solution (when applicable) may be at a pH comprised, for example, between 4.7 and 9.0, preferably between 5.7 and 8.0 and more preferably between 5.7 and 6.7) However a preferred aqueous media is 10 mM MES buffer at a pH of 6.5,

f) loading (contacting, charging) said aqueous solution of proteins containing a PSP94-binding protein in an ion-exchange (anion-exchange) chromatography column containing an ion-exchange (anion-exchange) chromatography matrix (resin, gel),

g) adding a salt solution selected from the group consisting of sodium chloride, magnesium chloride, potassium chloride to recover (elute, detach) proteins containing a PSP94-binding protein from said ion-exchange chromatography column, preferably sodium chloride with a molarity ranging from, for example, 100 mM to 1000 mM,

h) recovering a fraction (peak) of proteins containing a PSP94-binding protein,

i) contacting (charging, passing through) a PSP94-conjugated affinity matrix with the fraction recovered in order to generate a PSP94-conjugated affinity matrix bound to a PSP94-binding protein,

j) adding an eluting reagent (free PSP94, urea, sodium acetate or CAPS; preferably free PSP94) to said PSP94-conjugated affinity matrix bound to a PSP94-binding protein to recover (elute, detach) a PSP94-binding protein,

k) recovering a fraction containing a PSP94-binding protein,

l) loading said PSP94-binding protein in a size exclusion chromatography column containing a size exclusion chromatography matrix to separate PSP94-binding protein from contaminants, and;

m) recovering a fraction containing a (substantially) pure PSP94-binding protein.

It is to be understood that some of the purification steps described herein may prove to be unnecessary depending on the level of purification required or depending on the optimization of one or more of the remaining steps.

In a further aspect, the present invention relates to the product obtained from the purification process defined above.

In accordance with the present invention, samples (e.g., biological sample) referred herein may comprise, for example, blood, plasma, serum, urine, seminal fluid, cell culture media, cell lyzate, etc. The sample is preferably a human (e.g., male) sample.

In another aspect, the present invention relates to an antibody, and antigen binding fragments thereof, able to recognize a PSP94 epitope (i.e., exposed epitope) that is available even when PSP94 is bound to another polypeptide (another molecule). Such polypeptide may be for example, a polypeptide selected from the group consisting of SEQ ID NO.: 2, SEQ ID NO.: 3, SEQ ID NO.: 7, SEQ ID NO.:8, SEQ ID NO.:9, variant, fragment, analogue and combination thereof. The hybridoma cell line producing such antibody is also contemplated by the present invention. An example of such antibody is the monoclonal antibody produced by the hybridoma cell line deposited to the ATCC under Patent Deposit NO.: PTA-4241 (P1E8) or a polyclonal antibody able to recognize free and bound forms of PSP94.

The identification of an exposed epitope may be performed by testing a panel of antibody for their specificity to free and bound forms of PSP94. Antibodies which react (recognize) with both forms may represent candidate antibodies. In parallel, partial trypsin digestion may be performed on the PSP94/PSP94-binding protein complex. PSP94 epitopes (e.g., linear epitopes) available in the complexed forms may then be identified by amino acid sequence analysis. Antibodies able to bind to this or these (available) epitope(s) may be generated. Exposed epitopes are to be understood herein, as epitopes of a molecule (e.g., PSP94, SEQ ID NO.:2, SEQ ID NO.:3. SEQ ID NO.: 7, SEQ ID NO.:8, SEQ ID NO.:9 and their complex) that are accessible to an antibody, preferably when the molecule(s) or complex is in its native (natural) state (e.g., non-denatured, natural or 3D form).

In a further aspect, the present invention provides a method for removing PSP94 from a sample, said method comprising

a) contacting said sample with a molecule able to bind to PSP94 (the molecule may be directly or indirectly bound to a matrix or solid support) and

b) recuperating a sample free of PSP94.

It may proved useful to remove PSP94 from a sample (biological sample) for example, removing excess PSP94 from serum of individuals (i.e., serum depletion of PSP94) having elevated levels of PSP94 and to reinfuse a depleted serum into the individual (e.g., patient in need). In other instance, it may be useful to remove PSP94 from a sample in order to optimize measurement of other serum constituents. Removal of PSP94 is based on the affinity between PSP94 and any one of the sequence set forth in SEQ ID NO.: 2, SEQ ID NO.: 3, SEQ ID NO.: 7, SEQ ID NO.: 8, SEQ ID NO.: 9, PSP94 antibodies, and combination thereof.

The molecule referred above may molecule may be selected from the group consisting of SEQ ID NO.: 2, SEQ ID NO.:3, SEQ ID NO.: 7, SEQ ID NO.:8, SEQ ID NO.: 9, a monoclonal antibody produced by the hybridoma cell line deposited to the ATCC under Patent Deposit No.: PTA-4240 and a monoclonal antibody produced by the hybridoma cell line deposited to the ATCC under Patent Deposit No.: PTA-4241.

In yet a further aspect, the present invention provides a method for removing a complex formed by PSP94 and any one of the polypeptide defined in SEQ ID NO: 2, SEQ ID NO.:3, SEQ ID NO.:7, SEQ ID NO.:8, SEQ ID NO.:9 and combination thereof (e.g., PSP94/SEQ ID NO:2 and/or PSP94/SEQ ID NO.:3 and/or PSP94/SEQ ID NO:7, etc.) from a sample, said method comprising;

a) contacting said sample with an antibody able to recognize an available (exposed) epitope of said complex (e.g., the antibody may be directly or indirectly bound to a matrix or solid support) and

b) recuperating a sample free of said complex.

In one embodiment of the present invention, the antibody used in step b) may comprise, for example, a monoclonal antibody produced by the hybridoma cell line deposited to the ATCC under Patent Deposit No.: PTA-4241, a monoclonal antibody produced by the hybridoma cell line deposited to the ATCC under Patent Deposit No.: PTA-4242 and a monoclonal antibody produced by the hybridoma cell line deposited to the ATCC under Patent Deposit No.: PTA-4243. Preferably used is the monoclonal antibody produced by the hybridoma cell line deposited to the ATCC under Patent Deposit No.: PTA-4243.

Other aspects of the present invention encompass the monoclonal antibody produced by the hybridoma cell line deposited to the ATCC under Patent Deposit (e.g., Accession) No.: PTA-4240, as well as the monoclonal antibody produced by the hybridoma cell line deposited to the ATCC under Patent Deposit (e.g., Accession) No.: PTA-4241 and antigen binding fragments thereof.

Also covered by the present invention are the hybridoma cell lines producing the antibodies described herein. These include the hybridoma cell line deposited to the ATCC under Patent Deposit (e.g., Accession) No.: PTA-4240 and the hybridoma cell line deposited to the ATCC under Patent Deposit (e.g., Accession) No.: PTA-4241.

In another aspect, the present invention provides a method for measuring, in a sample, the total amount of PSP94, said method may comprise contacting said sample with an antibody able to recognize PSP94 even when PSP94 is bound to another polypeptide (such as for example, SEQ ID NO.:2, SEQ ID NO.:3. SEQ ID NO.:7, SEQ ID NO.:8, SEQ ID NO.:9 variants, fragments and analogues). This aspect of the invention encompasses any method which comprises this step, irrelevant of the fact that one or more steps are to be performed or not.

In one embodiment, the antibody that may be used in measuring the total amount of PSP94 in a sample, may be, for example, the monoclonal antibody produced by the hybridoma cell line deposited to the ATCC under Patent Deposit No.: PTA-4241 or it may be a polyclonal antibody able to recognize free and bound forms of PSP94.

The method for measuring total (free (unbound) and bound) amount of PSP94 in a sample contemplated above may comprise the following steps;

a) immobilizing (coating, adsorbing) a PSP94-antibody to a suitable substrate (ELISA plate, matrix, SDS-PAGE, Western blot membranes). The antibody may be able to recognize PSP94 even when bound to a PSP94-binding protein (such as SEQ ID NO.:2, SEQ ID NO.:3, SEQ ID NO.:7, SEQ ID NO.:8, SEQ ID NO.:9);

b) adding a sample comprising PSP94,

c) adding a PSP94 detection reagent comprising a label or marker, and;

d) detecting a signal resulting from a label or marker.

Examples of suitable detection reagents that may be used in step c) of the present method, include an antibody and a polypeptide having an affinity for PSP94. However, the detection reagent may preferably have a different binding site than the PSP94-antibody and a PSP94-binding protein. The detection reagent may either be directly coupled to a label (or marker) (e.g., antibody conjugate of the present invention) or able to be recognized by a second molecule carrying (conjugated with) said label or marker.

An example of a PSP94-antibody that may be used in step a) is the antibody (P1E8) produced by the hybridoma cell line deposited to the ATCC under Patent Deposit no.: PTA-4241. In that case, the detection reagent may be, for example, the antibody (2D3) (e.g., antibody-conjugate) produced by the hybridoma cell line deposited to the ATCC under Patent Deposit no.: PTA-4240 or any other suitable PSP94 antibody.

It is to be understood herein that a polyclonal antibody (one or more polyclonal antibodies) able to recognize free and bound forms of PSP94 may be suitable for any of steps a) or c) in combination with any of the monoclonal antibody described herein. For example, total PSP94 may be captured with a polyclonal antibody (an antibody able to recognize free and bound forms of PSP94) and detection may be performed (directly or indirectly) with another antibody such as P1E8 (and vice versa).

In addition, total PSP94 may be captured with an antibody able to recognize PSP94 in its free and bound forms (e.g., bound to a PSP94-binding protein as described herein), such as, for example, a polyclonal antibody or the P1E8 antibody (produced by the hybridoma cell line PTA-4241), and detection of the captured proteins (complex) may be performed with a combination of two or more antibodies i.e., one able to detect the free PSP94 (e.g., 2D3 produced by hybridoma cell line PTA-4240) and one or more antibodies able to detect PSP94-binding protein (e.g., 17G9 produced by the hybridoma cell line PTA-4243; and/or 3F4 produced by the hybridoma cell line PTA-4242).

In yet another aspect, the present invention provides an improved method for measuring the amount of free PSP94 in a sample, said method comprising contacting said sample with an antibody able to recognize PSP94 (e.g., in its free form).

In an embodiment of the present invention, suitable antibodies may include for example, the monoclonal antibody produced by the hybridoma cell line deposited to the ATCC under Patent Deposit No.: PTA-4240 and the monoclonal antibody produced by the hybridoma cell line deposited to the ATCC under Patent Deposit No.: PTA-4241. However, other suitable antibodies are encompassed by the present invention, such as the 12C3 antibody (Table 10).

In an additional aspect, the present invention provides an improved method for measuring the amount of free (unbound PSP94) PSP94 (and/or PSP94 fragments and analogues thereof) in a sample, said method comprising, contacting a sample free of the PSP94/PSP94-binding protein complex with an antibody able to recognize PSP94, PSP94 fragments and analogues thereof. For example, the improved method may for measuring the amount of free PSP94 in a sample may comprise;

a) removing a complex formed by PSP94 and any one of the polypeptide selected from the group consisting of SEQ ID NO.: 2, SEQ ID NO.:3, SEQ ID NO.:7 SEQ ID NO.:8, SEQ ID NO.:9 and combination thereof, generating a complex-free sample, and;

b) contacting said complex-free sample with an antibody able to recognize PSP94.

The improved method for measuring the amount of free (unbound PSP94) PSP94 in a sample contemplated herein may also comprise, for example, the following steps;

a) removing a complex formed by PSP94 and any one of the polypeptide selected from the group consisting of SEQ ID NO.: 2, SEQ ID NO.:3, SEQ ID NO.:7, SEQ ID NO.:8, SEQ ID NO.:9 variants, fragments analogues and combination thereof, generating a complex-free sample (e.g., using methods described herein)

b) immobilizing (coating, adsorbing) a PSP94-antibody to a suitable substrate (ELISA plate, matrix, SDS-PAGE, Western blot membranes),

c) adding said complex-free sample comprising free (unbound) PSP94,

d) adding a (PSP94) detection reagent comprising a label or marker, and;

e) detecting a signal resulting from a label or marker.

Examples of suitable detection reagents that may be used in the present invention are reagents selected from the group consisting of an antibody and a polypeptide having an affinity for PSP94. The detection reagent may have a different binding site than the PSP94-antibody, and the detection reagent may either be directly coupled to a label (or marker) or able to be recognized by a second molecule carrying (conjugated with) said label or marker.

An example of a PSP94-antibody used in step b) is the monoclonal antibody (2D3) produced by the hybridoma cell line deposited to the ATCC under Patent Deposit no.: PTA-4240. In that case, the monoclonal antibody (P1E8) (e.g., conjugated) produced by the hybridoma cell line deposited to the ATCC under Patent Deposit no.: PTA-4241 may be used as a detection reagent (directly or indirectly as described herein).

Another example of a PSP94-antibody that may be used in step b) is the monoclonal antibody (P1E8) produced by the hybridoma cell line deposited to the ATCC under Patent Deposit no.: PTA-4241. In that case the monoclonal antibody (2D3) (e.g., conjugated) produced by the hybridoma cell line deposited to the ATCC under Patent Deposit no.: PTA-4240 may be used as a detection reagent (directly or indirectly as described herein).

In a further aspect, the present invention relates to a method for measuring the amount of total PSP94 (bound and unbound (free)) in a sample, the method may comprise using a first and a second antibody able to bind to PSP94 even when PSP94 is bound to another polypeptide (e.g., SEQ ID NO.:2, SEQ ID NO.:3, SEQ ID NO.:7, SEQ ID NO.:8, SEQ ID NO.:9). It may be preferable that the first and second antibodies bind to a different PSP94 epitope.

In yet a further aspect, the present invention relates also to a method for measuring total PSP94 in a sample, the method comprising using a first and a second antibody, wherein said first antibody is able to bind to PSP94 even when PSP94 is bound to a polypeptide and wherein said second antibody is able to bind to PSP94 and to displace any one of the polypeptide selected from the group consisting of SEQ ID NO.:2, SEQ ID NO.:3, SEQ ID NO.:7, SEQ ID NO.:8, SEQ ID NO.:9 from a complex formed by PSP94 and said polypeptide.

In an embodiment of the present invention, the first antibody may be, for example, the monoclonal antibody produced by the hybridoma cell line deposited to the ATCC under Patent Deposit No.: PTA-4241, or any other suitable antibody. The second antibody may be, for example, the monoclonal antibody produced by the hybridoma cell line deposited to the ATCC under Patent Deposit No.: PTA-4240.

In an additional aspect the present invention provides a method for measuring the level (amount, concentration) of PSP94 in a sample said method comprising contacting said sample with an antibody that is able to recognize PSP94 in its free and bound forms (e.g., bound to SEQ ID NO.:2, SEQ ID NO.:3, SEQ ID NO.:7, SEQ ID NO.:8, SEQ ID NO.:9 etc.) forms.

In an embodiment of the present invention, the monoclonal antibody produced by the hybridoma cell line deposited to the ATCC under Patent Deposit NO.: PTA-4241 may be used.

When methods (e.g., measuring total PSP94, free PSP94, free or total PSP94-binding protein and calculating ratios) described herein are applied to clinical samples (serum, blood, plasma, etc.), they may be useful for screening subjects for a condition linked to abnormal or elevated levels of PSP94 (e.g., prostate cancer (e.g., prediction of relapse free interval in post-radiotherapy prostate cancer)) and for assessing, for example, prognosis in a subject diagnosed with prostate cancer. For example, it may be found that the higher the level of total PSP94 (or ratio of free PSP94/total PSP94, or total PSP94-binding protein) in individual with prostate cancer, relative to control subjects, the poorer the prognosis or higher the chance of having (developed recurrent) prostate cancer. In addition, when a raised level of total PSP94 (or other parameter described herein) is observed in a subject, it may be predictive (or suggestive) of prostate cancer in that subject. Thus, diagnostic and prognostic methods for screening subject for prostate cancer (or any other condition linked with an abnormal or elevated level of PSP94 or of PSP94-binding protein) are also encompassed by the present invention.

If desired or necessary, methods of the present invention may also include a step of collecting a sample; for example, a blood sample from an individual with a condition linked with elevated levels of PSP94 or other condition and performing the above-mentioned methods and assays.

Methods of the present invention may further comprise detecting a signal from a label that is provided (carried) by said molecule (antibody, polypeptide; e.g., from the label attached to the molecule) or by a second molecule (antibody or binding/ligand system) carrying said label.

Methods of the present invention may also include comparing (detecting) the signal (results) obtained for the sample with signal (results) obtained for a control sample containing a known amount of the polypeptide of interest.

In a further aspect, the present invention relates to the use of a PSP94 antibody for the treatment of a condition associated with elevated levels of PSP94. It is to be understood that a method of treating a patient with such condition, comprising administering a PSP94 antibody is also encompassed herein.

In yet a further aspect, the present invention relates to the use of a PSP94 antibody in the manufacture of a medicament for the treatment of a condition associated with elevated levels of PSP94.

The PSP94 antibodies may be for example, a monoclonal antibody produced by the hybridoma cell line deposited to the ATCC under Patent Deposit No.: PTA-4240 or a monoclonal antibody produced by the hybridoma cell line deposited to the ATCC under Patent Deposit No.: PTA-4241.

A sample, is to be understood herein as an aliquot of blood, serum, plasma, biological fluid, or it may be, for example, proteins (containing other constituents or not) bound to the well of an ELISA plate, a membrane, a gel, a matrix, etc.

In yet a further aspect, the present invention relates to the use of a molecule selected from the group consisting of the polypeptide as set forth in SEQ ID NO.:2, SEQ ID NO.:3, SEQ ID NO.: 7, SEQ ID NO.:8, SEQ ID NO.:9, a monoclonal antibody (2D3) produced by the hybridoma cell line deposited to the ATCC under Patent Deposit No.: PTA-4240, a monoclonal antibody (P1E8) produced by the hybridoma cell line deposited to the ATCC under Patent Deposit No.: PTA-4241, a monoclonal antibody (3F4) produced by the hybridoma cell line deposited to the ATCC under Patent Deposit No.: PTA-4242 and a monoclonal antibody (17G9) produced by the hybridoma cell line deposited to the ATCC under Patent Deposit No.: PTA-4243, for evaluating the amount of PSP94 (free and/or bound and/or total), PSP94 variants and analogues thereof in a sample.

According to the present invention, Conditions that are contemplated for methods and uses described herein may comprise, for example, prostate cancer, stomach cancer, breast cancer, endometrial cancer, ovarian cancer, other cancers of epithelial secretory cells and benign prostate hyperplasia (BPH).

It is to be understood herein that other antibody may be used (are suitable) in the methods described herein. For example, PSP94-binding protein specific antibodies listed in table 10 are interchangeable and are encompassed by the present invention (including their hydridoma cell lines). For example the monoclonal antibody (3F4) produced by the hybridoma cell line deposited to the ATCC under Patent Deposit NO.: PTA-4242 may be interchanged with the monoclonal antibodies 2B10, 9B6, 1B11, etc. and the monoclonal antibody (17G9) produced by the hybridoma cell line deposited to the ATCC under Patent Deposit NO.: PTA-4243 may be interchanged with the monoclonal antibody P8C2, 1B11, 26B10, 9B6, etc. A variety of other conditions are possible. However, when two antibodies are needed to perform the present methods it is preferable to choose antibodies that bind to different epitopes.

It is also to be understood herein that antibody fragments, such as an antigen-binding fragment (e.g., antigen binding site) of any of the (monoclonal) antibodies disclosed herein are encompassed by the present invention.

General Molecular Biology and Definitions

Unless otherwise indicated, the recombinant DNA techniques utilized in the present invention are standard procedures, known to those skilled in the art. Example of such techniques are explained in the literature in sources such as J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present) and are incorporated herein by reference.

“Polynucleotide” generally refers to any polyribonucleotide or polydeoxyribonucleotide, which may be unmodified RNA or DNA, or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus “polynucleotide” embraces chemically, enzymatically or metabolically modified forms of polynucleotides as typically found in nature, as well as the chemical forms of DNA and RNA characteristic of viruses and cells. “Polynucleotide” includes but is not limited to linear and end-closed molecules. “Polynucleotide” also embraces relatively short polynucleotides, often referred to as oligonucleotides.

Therefore, in accordance with the present invention, the polynucleotide may be, for example, a polyribonucleotide, a polydeoxyribonucleotide, a modified polyribonucleotide, a modified polydeoxyribonucleotide, a complementary polynucleotide (e.g., antisense) or a combination thereof.

“Polypeptide” refers to any peptide or protein comprising two or more amino acids joined to each other by peptide bonds or modified peptide bonds (i.e., peptide isosteres). “Polypeptide” refers to both short chains, commonly referred as peptides, oligopeptides or oligomers, and to longer chains generally referred to as proteins. As described above, polypeptides may contain amino acids other than the 20 gene-encoded amino acids.

“Variant” as the term used herein, is a polynucleotide or polypeptide that differs from reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion and truncations in the polypeptide encoded by the reference sequence, as discussed herein. A typical variant of a polypeptide differs in amino acid sequence from another, reference polypeptide. Generally, differences are limited so that the sequence of the reference polypeptide and the variant are closely similar overall and, in many regions, identical. A variant and reference polypeptide may differ in amino acid by one or more substitutions, additions, deletions, or any combination therefore. A substituted or inserted amino acid residue may or may not be one encoded by the genetic code. A variant polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. “Variants” as used herein encompass (active) mutants, analogues, homologues, chimeras, fragments and portions thereof. However, “variants” as used herein may retain parts of the biological activity of the original polypeptide.

As used herein, “pharmaceutical composition” means therapeutically effective amounts of the agent together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

An “immunizing composition” or “immunogenic composition” as used herein refers to a composition able to promote an immune response in the host receiving such composition. An “immunizing composition” includes a compound, such as for example, a polypeptide (or a DNA or RNA able to encode a polypeptide) for which an antibody is sought. The polypeptide is usually diluted in a buffer, diluent or a pharmaceutically acceptable carrier. An “immunizing composition” may comprise an adjuvant such as or example complete Freund's adjuvant, incomplete Freund's adjuvant and aluminum hydroxide.

Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

As used herein, “PSP94-binding protein” relates to a protein (such as SEQ ID NO.: 2, SEQ ID No.: 3, SEQ ID NO.:7, SEQ ID NO.: 8, SEQ ID NO.: 9) that is able to bind (i.e., associate) to PSP94, usually in a reversible fashion.

As used herein, the term “free PSP94” relates to a PSP94 protein that is not associated with another polypeptide. The term “free PSP94” means that PSP94 is in an unbound form (state).

As used herein, the term “antibody” refers to either monoclonal antibody, polyclonal antibody, humanized antibody, single-chain antibody, antibody fragments including Fc, F(ab)2, F(ab)2′ and Fab and the like.

As used herein, the term “antigen binding fragment” relates to an antibody fragment (antigen binding domain) able to recognize (bind) the antigen of interest. An “antigen binding fragment”, may be isolated from the gene(s) (e.g., gene encoding a variable region) encoding the antibody using molecular biology methods. The isolated gene(s) may engineered to create, for example, a single chain antibody.

As used herein “PSP94” relates to the native and recombinant PSP94.

Gene (cDNA) Cloning and Protein Expression

The identified and isolated gene (i.e., polynucleotide) may be inserted into an appropriate cloning or expression vector (i.e., expression system). A large number of vector-host systems known in the art may be used. Possible vectors include, but are not limited to, plasmids or modified viruses (e.g., bacteriophages, adenoviruses, adeno-associated viruses, retroviruses), but the vector system must be compatible with the host cell used. Examples of cloning vectors include, but are not limited to, Escherichia coli (E. coli), bacteriophages such as lambda derivatives, or plasmids such as pBR322 derivatives or pUC plasmid derivatives (e.g., pGEX vectors, pmal-c, pFLAG, etc). Examples of expression vectors are discussed bellow. The insertion into a cloning or expression vector can, for example, be accomplished by ligating the DNA fragment into a cloning vector, which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, lipofection, infection, electroporation, etc. The cloned gene may be contained on a shuttle vector plasmid, which provides for expansion in a cloning cell, e.g., E. coli, and facilitate purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2. mu. plasmid.

It is to be understood herein that when the polynucleotide (e.g., gene, cDNA, RNA) of the present invention is inserted into the appropriate vector, it may be used, for example, as a way to express the protein in a foreign host cell for its isolation (such as bacteria, yeast, insect, animal or plant cells) or in a (isolated) cell from an individual for purpose of gene therapy treatment or cell-mediated vaccination (using, for example, dendritic cells). For example, cells may be isolated from a mammal and treated (e.g., exposed, transfected, lipofected, infected, bombarded (using high velocity microprojectiles)) ex-vivo with the polynucleotide (cDNA, gene, RNA, antisense) of the present invention before being re-infused in the same individual or in a compatible individual. In vivo delivery of a polynucleotide may be performed by other methods than the one described above. For example, liposomal formulations when injected, may also be suitable for mediating in vivo delivery of a polynucleotide.

Any of a wide variety of expression systems may be used to provide a recombinant polypeptide (protein). The precise host cell used is not critical to the invention. Polypeptides of the present invention may be produced in a prokaryotic host (e.g., E. coli or Bacillus subtilis (B. subtilis)) or in a eukaryotic host (yeast e.g., Saccharomyces or Pichia Pastoris; mammalian cells, e.g., monkey COS cells, mouse 3T3 cells (Todaro G J and Green H., J. Cell Biol. 17: 299-313, 1963), Chinese Hamster Ovary cells (CHO) (e.g., Puck T T et al., J. Exp. Med. 108: 945-956, 1958), BHK, human kidney 293 cells (e.g., ATCC: CRL-1573), or human HeLa cells (e.g., ATCC:CCL-2); or insect cells).

In a yeast cell expression system such as Pichia Pastoris (P. Pastoris), DNA sequence encoding polypeptides of the present invention may be cloned into a suitable expression vector such as the pPIC9 vector (Invitrogen). Upon introduction of a vector containing the DNA sequence encoding all or part of the polypeptides of the present invention into the P. Pastoris host cells, recombination event may occur for example in the AOX1 locus. Such recombination event may place the DNA sequence of polypeptides of the present invention under the dependency of the AOX1 gene promoter. Successful insertion of a gene (i.e. DNA sequence) encoding polypeptides of the present invention may result in an expression of such polypeptides that is regulated and/or induced by methanol added in the growth media of the host cell (for reference see Buckholz, R. G. and Gleeson, M. A. G., Biotechnology, 9:1067-1072,1991; Cregg, J. M., et al., Biotechnology, 11:905-910, 1993; Sreekrishna, K., et al., J. Basic Microbiol., 28:265-278, 1988; Wegner, G. H., FEMS Microbiology Reviews, 87:279-284, 1990).

In mammalian host cells, a number of viral-based expression systems may be utilized. For example, in the event where an adenovirus is used as an expression vector for the polypeptides of the present invention, nucleic acid sequence may be ligated to an adenovirus transcription/translation control complex (e.g., the late promoter and tripartite leader sequence). This chimeric gene may be inserted into the adenovirus genome, for example, by in vitro or in vivo recombination. Insertion into a non-essential region of the viral genome (e.g., region E1 or E3) may result in a recombinant virus that is viable and capable of expressing polypeptides of the present invention in infected hosts.

Proteins and polypeptides of the present invention may also be produced by plant cells. Expression vectors such as cauliflower mosaic virus and tobacco mosaic virus and plasmid expression vectors (e.g., Ti plasmid) may be used for the expression of polypeptides in plant cells. Such cells are available from a wide range of sources (e.g., the American Type Culture Collection, Rockland, Md.). The methods of transformation or transfection and the choice of expression vehicle are of course to be chosen accordingly to the host cell selected.

In an insect cell expression system such as Autographa californica nuclear polyhedrosis virus (AcNPV), which grows in Spodoptera frugiperda cells, AcNPV may be used as a vector to express foreign genes. For example, DNA sequence coding for polypeptides of the present invention may be cloned into non-essential regions of the virus (for example the polyhedrin gene) and placed under control of an AcNPV promoter, (e.g., the polyhedrin promoter). Successful insertion of a gene (i.e., DNA sequence) encoding polypeptides of the present invention may result in inactivation of the polyhedrin gene and production of non-occluded recombinant virus (i.e., virus lacking the proteinaceous coat encoded by the polyhedrin gene). These recombinant viruses may be used to infect spodoptera frugiperda cells in which the inserted gene is expressed.

In addition, a host cell may be chosen for its ability to modulate the expression of the inserted sequences, or to modify or process the gene product in a specific, desired fashion. Such modifications (e.g., glycosylation) and processing (e.g., cleavage) of protein products may be important for the function of the protein. Different host cells have characteristics and specific mechanisms for posttranslational processing and modification of proteins and gene products. Of course, cell lines or host systems may be chosen to ensure desired modification and processing of the foreign protein expressed. To this end, eukaryotic host cells that possess the cellular machinery for proper processing of the primary transcript, glycosylation, and phosphorylation of the gene product may be used. Such mammalian host cells comprise for example, but are not limited to, CHO, VERO, BHK, HeLa, COS, MDCK, 293, and 3T3.

Alternatively, polypeptides of the present invention may be produced by a stably-transfected mammalian cell line. A number of vectors suitable for stable transfection of mammalian cells are available to the public; methods for constructing such cell lines are also publicly available. In one example, cDNA encoding the rHuPSP94 protein may be cloned into an expression vector that includes the dihydrofolate reductase (DHFR) gene. Integration of the plasmid and, therefore, DNA sequence of polypeptides of the present invention, into the host cell chromosome may be selected for by including methotrexate in the cell culture media. This selection may be accomplished in most cell types.

Specific initiation signals may also be required for the efficient translation of DNA sequences inserted in a suitable expression vehicle as described above. These signals may include the ATG initiation codon and adjacent sequences. For example, in the event where gene or cDNA encoding polypeptides of the present invention, would not have their own initiation codon and adjacent sequences, additional translational control signals may be needed. For example, exogenous translational control signals, including, perhaps, the ATG initiation codon, may be needed. It is known in the art that the initiation codon must be in phase with the reading frame of the polypeptide sequence to ensure proper translation of the desired polypeptide. Exogenous translational control signals and initiation codons may be of a variety of origins, including both natural and synthetic. The efficiency of expression may be enhanced by the inclusion of appropriate transcription enhancer elements, transcription terminators. The transcription, translation signals may be specifically engineered to provide a desired expression pattern and level (e.g., signals that may require a specific inducer, signals that will allow expression in a defined cell type or in a specific time frame). However, these signals may be provided by the expression vector, which often contains a promoter enabling the expression of the polypeptide in a desired host cell.

Polypeptide Modifications (Mutants, Variants, Analogues, Homologues Chimeras and Portions/Fragments).

As may be appreciated, a number of modifications may be made to the polypeptides and fragments of the present invention without deleteriously affecting the biological activity of the polypeptides or fragments. Polypeptides of the present invention comprises for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino or carboxy-termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2^{nd} Ed., T. E. Creighton, W. H. Freeman and Company, New-York, 1993).

Other type of polypeptide modification may comprises for example, amino acid insertion (i.e., addition), deletion and substitution (i.e., replacement), either conservative or non-conservative (e.g., D-amino acids, desamino acids) in the polypeptide sequence where such changes do not substantially alter the overall biological activity of the polypeptide. Polypeptides of the present invention comprise for example, biologically active mutants, variants, fragments, chimeras, and analogs; fragments encompass amino acid sequences having truncations of one or more amino acids, wherein the truncation may originate from the amino terminus (N-terminus), carboxy terminus (C-terminus), or from the interior of the protein. Polypeptide analogs of the invention involve an insertion or a substitution of one or more amino acids. Variants, mutants, fragments, chimeras and analogs may have the biological property of polypeptides of the present invention.

It should be further noted that if the polypeptides are made synthetically, substitutions by amino acids, which are not naturally encoded by DNA may also be made. For example, alternative residues include the omega amino acids of the formula NH2(CH2)nCOOH wherein n is 2-6. These are neutral nonpolar amino acids, as are sarcosine, t-butyl alanine, t-butyl glycine, N-methyl isoleucine, and norleucine. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

It is known in the art that mutants or variants may be generated by substitutional mutagenesis and retain the biological activity of the polypeptides of the present invention. These variants have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in table 1. If such substitutions result in a change not desired, then other type of substitutions, denominated “exemplary substitutions” in table 1, or as further described herein in reference to amino acid classes, are introduced and the products screened.

Example of substitutions may be those, which are conservative (i.e., wherein a residue is replaced by another of the same general type). As is understood, naturally-occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same group as that of the amino acid be replaced. Thus, in some cases, the basic amino acids Lysine (Lys), Arginine (Arg) and Histidine (His) may be interchangeable; the acidic amino acids Aspartic acid (Asp) and Glutamic acid (Glu) may be interchangeable; the neutral polar amino acids Serine (Ser), Threonine (Thr), Cysteine (Cys), Glutamine (Gln), and Asparagine (Asn) may be interchangeable; the non-polar aliphatic amino acids Glycine (Gly), Alanine (Ala), Valine (Val), Isoleucine (Ile), and Leucine (Leu) are interchangeable but because of size Gly and Ala are more closely related and Val, Ile and Leu are more closely related to each other, and the aromatic amino acids Phenylalanine (Phe), Tryptophan (Trp) and Tyrosine (Tyr) may be interchangeable.

TABLE 1
Preferred amino acid substitution

| | | Conservative |
|---|---|---|
| Original residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, | Leu |
| | Phe, norleucine | |
| Leu (L) | Norleucine, Ile, Val, | Ile |
| | Met, Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, | Leu |
| | Ala, norleucine | |

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:

(1) hydrophobic: norleucine, methionine (Met), Alanine (Ala), Valine (Val), Leucine (Leu), Isoleucine (Ile)

(2) neutral hydrophilic: Cysteine (Cys), Serine (Ser), Threonine (Thr)

(3) acidic: Aspartic acid (Asp), Glutamic acid (Glu)

(4) basic: Asparagine (Asn), Glutamine (Gln), Histidine (His), Lysine (Lys), Arginine (Arg)

(5) residues that influence chain orientation: Glycine (Gly), Proline (Pro); and

(6) aromatic: Tryptophan (Trp), Tyrosine (Tyr), Phenylalanine (Phe)

Non-conservative substitutions will entail exchanging a member of one of these classes for another.

Mutant polypeptides will possess one or more mutations, which are deletions (e.g., truncations), insertions (e.g., additions), or substitutions of amino acid residues. Mutants can be either naturally occurring (that is to say, purified or isolated from a natural source) or synthetic (for example, by performing site-directed mutagenesis on the encoding DNA or made by other synthetic methods such as chemical synthesis). It is thus apparent that the polypeptides of the invention can be either naturally occurring or recombinant (that is to say prepared from the recombinant DNA techniques).

A protein at least 50% identical, as determined by methods known to those skilled in the art (for example, the methods described by Smith, T. F. and Waterman M. S. (1981) Ad. Appl. Math., 2:482-489, or Needleman, S. B. and Wunsch, C. D. (1970) J. Mol. Biol., 48: 443-453), to those polypeptides of the present invention are included in the invention, as are proteins at least 70% or 80% and more preferably at least 90% identical to the protein of the present invention. This will generally be over a region of at least 5, preferably at least 20, contiguous amino acids.

Amino acid sequence variants may be prepared by introducing appropriate nucleotide changes into DNA, or by in vitro synthesis of the desired polypeptide. Such variant include, for example, deletions, insertions, or substitutions of residues within the amino acid sequence. A combination of deletion, insertion and substitution can be made to arrive at the final construct, provided that the final protein product possesses the desired characteristics. The amino acid changes also may alter posttranslational processes such as changing the number or position of the glycosylation sites, altering the membrane anchoring characteristics, altering the intra-cellular location by inserting, deleting or otherwise affecting the transmembrane sequence of the native protein, or modifying its susceptibility to proteolytic cleavage.

Protein Purification

Some aspects of the present invention concern the purification, and in particular embodiments, the substantial purification, of a polypeptide. The term “purified polypeptide” as used herein, is intended to refer to a composition, isolatable from other components, wherein the polypeptide is purified to any degree relative to its naturally-obtainable state, (i.e., in this case, relative to its purity within a prostate, cell extract). A purified polypeptide therefore also refers to a polypeptide, free from the environment in which it may naturally occur.

Generally, “purified” will refer to a polypeptide composition, which has been subjected to fractionation to remove various other components, and which composition substantially retains its expressed biological activity. Where the term “substantially purified” is used, this will refer to a composition in which the polypeptide forms the major component of the composition, such as constituting about 50% or more of the polypeptides in the composition.

Various techniques suitable for use in polypeptide purification will be well known to those of skill in the art. These include, for example, precipitation with ammonium sulfate, PEG, antibodies and the like or by heat denaturation, followed by centrifugation; chromatography steps such as ion exchange, gel filtration (i.e., size exclusion chromatography), reverse phase, hydroxylapatite and affinity chromatography; isoelectric focusing; gel electrophoresis; and combinations of such and other techniques. These techniques may be used either alone or in combination. As is generally known in the art, it is believed that the order of conducting the various purification steps may be changed, or that certain steps may be omitted, and still result in a suitable method for the preparation of a substantially purified polypeptide.

The ability of purifying a protein by ammonium sulfate precipitation is based on the fact that a protein's solubility is lowered at high salt concentration. However, the solubility of proteins is affected in a different manner depending on their properties.

Size exclusion chromatography or gel filtration separates molecules based on their size. The gel (i.e., matrix, resin) media may consist of beads containing pores of a specific distribution. Separation may occurs when molecules of different size are included or excluded from the pores within the matrix. Small molecules may diffuse into the pores and their flow through the column is retarded, while large molecules do not enter the pores and are eluted in the column's void volume. Consequently, molecules separate based on their size as they pass through the column and are eluted in order of decreasing molecular weight.

Proteins can be separated on the basis of their net charge by ion-exchange chromatography. For example, if a protein has a net positive charge at pH 7, it will usually bind (adsorb) to beads (i.e., matrix) containing a negatively charged group. For example, a positively charged protein can be separated on a negatively charged carboxymethyl-cellulose or carboxymethyl-agarose matrix. Following elution, proteins that have a low density of net positive charge will tend to emerge first from the column followed by those having a higher charge density. Negatively charged proteins can be separated by chromatography on positively charged diethylaminoethyl-cellulose (DEAE-cellulose) or DEAE-agarose matrix. A charged protein bound to an ion-exchange matrix may be eluted (released, detached) by increasing the concentration of sodium chloride or another salt solution as an eluting buffer. Ions will compete with the charged groups on the protein for binding to the matrix.

Salt solutions may be added to the matrix in a sequential manner (i.e., by adding a solution of a specific molarity (e.g., 100 mM sodium chloride) followed by the addition of one or more solutions of different molarity (e.g., 200 mM, followed by a solution of 300 mM, followed by a solution of 400 mM, followed by a solution of 500 mM, followed by a solution of 1000 mM)) until the specific polypeptide of the invention (i.e., PSP94-binding protein (SEQ ID NO.:2, SEQ ID NO.:3, SEQ ID NO.:7, SEQ ID NO.:8, SEQ ID NO.:9) is eluted. In addition, salts solution may be added as a continuous gradient. For example, a salt solution of high molarity (e.g., 1000 mM) may be gradually added to a second solution of lower molarity (e.g., 100 mM) before entering the ion-exchange chromatography column. The salt solution entering the column will have a molarity slowly increasing from 100 mM to up to 1000 mM.

Affinity chromatography may be used when the specificity (affinity) of a polypeptide for a compound is known or suspected. For example, as a first step such compound (e.g., PSP94) is covalently attached to a column (e.g., a cyanogen bromide activated sepharose matrix) and a mixture (solution) containing a desired polypeptide (e.g., a PSP94-binding protein) may be added to the matrix. After washing the matrix, to remove unbound proteins, the desired polypeptide may be eluted from the matrix by adding a high concentration of the compound (e.g., PSP94) in a soluble form. Antibodies are an example of a compound, which is often used to purify proteins to which it binds.

It is known in the art, that equilibration and substantial washing of chromatography matrix (i.e., resin) (e.g., ion-exchange matrix, size-exclusion matrix, affinity matrix) is preferred in order to minimize binding of unwanted (i.e., unspecific) proteins (non-specific binding).

Antibodies and Hybridoma

Other aspects of the present invention relates to antibodies and hybridoma cell lines. The preparation and characterization of antibodies are well known in the art (See, e.g., Antibodies: A Laboratory Manual., Cold Spring Harbor Laboratory, 1988; incorporated herein by reference) and has been discussed in U.S. Pat. No. 6,156,515, the entire content of which is incorporated herein by reference.

For example, a polyclonal antibody preparation may be obtained by immunizing an animal with an immunogenic (immunizing) composition and collecting antisera from that immunized animal. A wide range of animal species may be used for the production of antisera. Typically the animal used for production of anti-antisera is a rabbit, a mouse, a rat, a hamster, a guinea pig or a goat.

It is often necessary to boost the host immune system by coupling, for example, an immunogen to a carrier (e.g., keyhole limpet hemocyanin (KLH) and bovine serum albumin (BSA)) or by incorporating an adjuvant to the immunizing composition, as described herein.

The production of antibodies may be monitored by sampling blood of the immunized animal at various time points following immunization. Sometimes, additional boosts may be required to provide a sufficient titer of the antibody(ies).

The desired antibody may be purified by known methods, such as affinity chromatography using, for example, another antibody or a peptide bound to a solid matrix.

Monoclonal antibodies (mAbs) may be readily prepared through use of known techniques, such as those exemplified in U.S. Pat. No. 4,196,265, the entire content of which is incorporated herein by reference. Mice (e.g., BALB/c mouse) and rats are the animals that are usually used for the immunization. Following immunization, B lymphocytes (B cells), are selected for use in the mAb generating protocol. Often, a panel of animals will have to be immunized and the animal having the highest antibody titer will be chosen. The antibody-producing B lymphocytes from the immunized animal are then fused (e.g., using polyethylene glycol) with cells of an immortal myeloma cell. Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 41, Sp210-Ag14, FO, NSO/JU, MPC-11, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

Fused hybrids are grown in a selective medium that enables the differentiation between fused cells and the parental cells (i.e., myeloma and B cells). The selective medium usually contains an agent (e.g., aminopterin, methotrexate, azaserine) that blocks the de novo synthesis of nucleotides. When aminopterin or methotrexate is used, the media is supplemented with hypoxanthine and thymidine as a source of nucleotides (HAT medium). Where azaserine is used, the media is supplemented with hypoxanthine. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells may operate this pathway, but they have a limited life span in culture and generally die within about two weeks. Therefore, the only cells that can survive in the selective media are those hybrids formed from myeloma and B cells.

Selection of hybridomas is performed by culturing the cells by single-clone dilution in microtiter plates, followed by testing the individual clonal supernatants for the desired reactivity. The selected hybridomas may then be serially diluted and cloned into individual antibody-producing cell lines, which clones may then be propagated indefinitely to provide mAbs.

Fragments of monoclonal antibody(ies) are encompassed by the present invention. These may be obtained by methods, which include digestion with enzymes such as pepsin or papain and/or cleavage of disulfide bonds by chemical reduction. Alternatively, monoclonal antibody fragments encompassed by the present invention may be synthesized using an automated peptide synthesizer or may be produced from cloned gene segments engineered to produce such fragment (e.g., single-chain antibody) in a suitable cell (cell line).

Antibody conjugates are also encompassed by the present invention. These may be generated by coupling the antibody with a fluorophore, a chromophore or dye (e.g., rhodamine, fluoroscein, and green fluorescent protein) or any other agent or label that gives rise to a detectable signal, either by acting alone or following a biochemical reaction (e.g., enzymes such as horseradish peroxidase, alkaline phosphatase and beta-galactosidase). A molecule such as diethylenetriaminepentaacetic acid (DTPA) may also be linked to the antibody. DTPA may act as a chelating agent that is able to bind to heavy metal ions including radioisotopes (e.g. Isotope 111 of Indium (¹¹¹In)). These conjugates may be used as detection tools in immunoassays or in imaging. Alternatively, conjugates having a therapeutic agent such as a toxin may be prepared from the monoclonal antibodies of the present invention, these may be used to target cancer cells and to promote their destruction.

It will be appreciated by those of skill in the art that monoclonal or polyclonal antibodies specific for proteins that are linked to prostate cancer will have utilities in several types of applications. These may include the production of diagnostic kits for use in detecting, diagnosing or evaluating the prognosis of individual with prostate cancer.

Antigen Detection

In terms of antigen detection, the biological sample analyzed may be any sample that is suspected of containing an antigen of interest, either a tissue, cell lysate, urine, blood, serum, plasma, etc.

Contacting the biological sample with the antigen detection (detecting) reagent (protein, peptide or antibody) is generally a matter of simply adding the composition to the sample and incubating the mixture for a period of time long enough for the antibodies to form immune complexes with the antigen. Washing of the sample (i.e., tissue section, ELISA plate, dot blot or Western blot) is generally required to remove any non-specifically bound antibody species. The antigen-antibody complex (immunocomplex) is then detected using specific reagents.

When, for example, the antigen detecting reagent is an antibody (a specific antibody), this antibody may be (directly) labeled with a marker (fluorophore, chromophore, dye, enzyme, radioisotope, etc.) for enabling the detection of the complex. In other instances, it may be advantageous to use a secondary binding ligand such as a secondary antibody or a biotin/avidin (streptavidin) (binding/ligand complex) arrangement, as is known in the art. Again, secondary antibodies may be labeled with a marker as described above or with an arrangement of biotin/avidin (i.e. avidin peroxidase), which allow the detection of the immunocomplex. United States Patents concerning the use of such labels include U.S. Pat. Nos. 3,817,837; 3,850,752; 3,939,350; 3,996,345; 4,277,437; 4,275,149 and 4,366,241, each incorporated herein by reference. Usually, the secondary antibody will be an antibody directed to the specific antibody (primary antibody) of a defined isotype and species such as, for example, an anti-mouse IgG.

On the other hand, the antigen detecting reagent may also be a polypeptide having affinity for an antibody or another polypeptide, which forms a complex (i.e., polypeptide-polypeptide complex or antibody-polypeptide complex). In that case, the polypeptide itself may be labeled using the markers described above, allowing direct detection. Again, the complex may be detected indirectly by adding a secondary (labeled) antibody or polypeptide.

Immunodetection methods, such as enzyme-linked immunosorbent assays (ELISA), Western blots, etc. have utility in the diagnosis of conditions such as prostate cancer. However, these methods also have applications to non-clinical samples, such as in the titering of antigen or antibody samples, in the selection of hybridomas, and the like.

ELISA

As noted, it is contemplated that the encoded polypeptides (SEQ ID NO.:2, SEQ ID No.:3, SEQ ID NO.:7, SEQ ID NO.:8, SEQ ID NO.:9) of the present invention will find utility in immunohistochemistry and in ELISA assays but also as immunogen (i.e., antigen) in connection with vaccine development. One evident utility of the encoded polypeptide and corresponding antibodies is in immunoassays for the diagnosis/prognosis of prostate cancer.

Immunoassays that may be performed using reagents (the polypeptide defined in SEQ ID NO.: 2, in SEQ ID NO.: 3, in SEQ ID NO.:7, in SEQ ID NO.:8 or in SEQ ID NO.:9 and antibodies) of the present invention includes, for example, enzyme linked immunosorbent assays (ELISAs) and radioimmunoassays (RIA), which are known in the art. Immunohistochemical detection using tissue sections is also particularly useful. However, it will be readily appreciated that detection is not limited to such techniques, and Western blotting, dot blotting, FACS analyses, and the like also may be used.

Examples of ELISA assays include the following; antibodies binding to a polypeptide (e.g., antibodies to PSP94 or antibodies to PSP94-binding protein (SEQ ID NO.:2, SEQ ID NO.: 3, etc.)) are immobilized onto a selected surface (i.e., suitable substrate) exhibiting protein affinity, such as a well in a polystyrene microtiter plate (ELISA plate). Then, a sample suspected of containing the polypeptide is added to the wells of the plate. After binding and washing to remove non-specifically bound immunocomplexes, the bound antigen may be detected. Detection may be achieved by the addition of a second antibody specific for the target polypeptide, which is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label (marker).

Another example of ELISA assay is the following; the samples suspected of containing the polypeptide of interest are immobilized onto the surface of a suitable substrate and then contacted with the antibodies of the invention. After binding and washing to remove non-specifically bound immunocomplexes, the bound antigen is detected. The immunocomplexes may be detected directly or indirectly as described herein.

An additional example of an ELISA assay is the following; again, polypeptides are immobilized to a substrate, however, in that case the assay involves a competition step. In this ELISA, a known amount of the polypeptide of interest is adsorbed to the plate. The amount of polypeptide in an unknown sample is then determined by mixing the sample with a specific antibody before or during incubation with wells containing the immobilized polypeptide. A detection reagent is added (e.g., antibody) to quantify the antibody that is able to bind to the immobilized polypeptide. The presence of the polypeptide in the sample acts to reduce the amount of antibody available for binding to the polypeptide contained in the well (immobilized polypeptide) and thus reduces the signal.

In order to get a correlation between the signal and the amount (concentration) of polypeptide in an unknown sample, a control sample may be included during the assay. For example, known quantities of a polypeptide (usually in a substantially pure form) may be measured (detected) at the same time as the unknown sample. The signal obtained for the unknown sample is then compared with the signal obtained for the control. The intensity (level) of the signal is usually proportional to the amount of polypeptide (antibody bound to the polypeptide) in a sample. However, the amount of control polypeptide and antibodies required to generate a quantitative assay needs to be evaluated first.

In coating a plate with either an antigen (polypeptide) or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

Conditions that may allow immunocomplex (antigen/antibody) formation include diluting the antigens and antibodies with solutions such as BSA, bovine gamma globulin (BGG) and phosphate buffered saline (PBS)/Tween. These added agents also tend to assist in the reduction of nonspecific background.

Suitable conditions involves that the incubation is at a temperature and for a period of time sufficient to allow effective binding. Incubation steps are typically from about 1 to 2 to 4 h, at temperatures preferably on the order of 20° C. to 27° C., or may be overnight at about 4° C. or so.

Often, the detection of the immunocomplex is performed with a reagent that is linked to an enzyme. Detections then requires the addition of the enzyme substrate. Enzymes such as, for example, alkaline phosphatase or peroxidase, when given an appropriate substrate will generate a reaction that may be quantified by measuring the intensity (degree) of color produced. The reaction is usually linear over a wide range of concentrations and may be quantified using a visible spectra spectrophotometer.

Kits

The present invention also relates to immunodetection kits and reagents for use with the immunodetection methods described above. As the polypeptide of the present invention may be employed to detect antibodies and the corresponding antibodies may be employed to detect the polypeptide, either or both of such components may be provided in the kit. The immunodetection kits may thus comprise, in suitable container means, a polypeptide (PSP94, or PSP94-binding protein), or a first antibody that binds to a polypeptide and/or an immunodetection reagent. The kit may comprise also a suitable matrix to which the antibody or polypeptide of choice may already be bound. Suitable matrix include an ELISA plate. The plate provided with the kit may already be coated with the antibody or polypeptide of choice. The coated ELISA plate may also have been blocked using reagents described herein to prevent unspecific binding. Detection reagents may also be provided and may include, for example, a secondary antibody or a ligand, which may carry the label or marker and/or an enzyme substrate. Kits may further comprise an antibody or polypeptide (usually of known titer or concentration) that may be used for control. Reagents may be provided, for example, lyophilized or in liquid form (of a defined concentration) and are provided in suitable containers (ensuring stability of reagents, safety etc.).

It is to be understood herein, that if a “range”, “group of substances” or particular characteristic (e.g., temperature, concentration, time and the like) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,

with respect to reaction time, a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;

and similarly with respect to other parameters such as concentrations, temperature, etc . . . .

It is also to be understood herein that non-PSP94-binding protein (or DNA encoding such polypeptide) are excluded of the polypeptide or polynucleotide of the present invention.

TABLE 2
Table of abbreviation.

| Abbreviation | Signification |
|---|---|
| M | Molar |
| mM | milliMolar |
| g | gram |
| mg | milligram |
| μg | microgram |
| ng | nanogram |
| ° C. or ° C. | Degree Celcius |
| % | percent |
| cm | centimeter |
| cpm (CPM) | Counts per minute |
| PBS | Phosphate buffered saline |
| NaCl | Sodium chloride |
| MES | 2-(N-Morpholino)ethanesulfonic acid |
| MOPS | 3-(N-Morpholino)propanesulfonic acid |
| UV | ultraviolet |
| Da | dalton |
| kDa | kilodalton |
| Kd | Dissociation constant |
| nm | nanometer |
| OD | Optical density |
| CAPS | 3-(Cyclohexylamino)-1-propanesulfonic |
| | acid |
| HMW | High molecular weight |
| LMW | Low molecular weight |
| FSH | Follicle stimulating hormone |
| PSP94 | Prostate Secretory Protein of 94 |
| | amino acids |
| SDS | Sodium dodecyl sulfate |
| PAGE | Polyacrylamide gel electrophoresis |
| DMSO | Dimethylsulfoxide |
| PVDF | Polyvinylidene difluoride |

The content of each publication, patent and patent application mentioned in the present application is incorporated herein by reference.

### BRIEF DESCRIPTION OF THE DRAWINGS

FIG. 1 is a graph showing size exclusion chromatography results of proteins from human male serum bound to PSP94 radiolabeled with isotope 125 of iodine (¹²⁵I) (specific binding). Binding of ¹²⁵I-PSP94 to human male serum protein is determined by the radioactivity, expressed in counts per minute (cpm), in each fraction. Non-specific binding was determined by including free PSP94 in the incubation mixture together with human male serum and ¹²⁵I-PSP94. The location of fractions containing free- and complexed-PSP94 (PSP94 associated with a carrier) are indicated in the graph.

FIG. 2 is a graph depicting results of ¹²⁵I-PSP94 binding in fractions of proteins, from human male serum, partially purified by ammonium sulfate precipitation. Whole human male serum was precipitated with various concentrations of ammonium sulfate (0 to 32%, 32 to 47%, 47 to 62% and 62 to 77% of ammonium sulfate (% are calculated in w/v)), and the presence of PSP94-binding activity within the fractions was assessed by measuring the ability of radiolabeled PSP94 to associate with proteins contain in each fraction (high molecular weight components) of serum. Results are expressed as the amount of radioactivity bound to human male serum proteins in each fraction relative to the total amount of radioactivity used in the binding assay (in terms of percentage).

FIG. 3 is a graph showing anion-exchange chromatography results using a MacroPrep High Q anion exchange column, loaded with proteins purified by ammonium sulfate. Proteins are eluted with sodium chloride. The peak located between point A and B represents the protein fraction containing PSP94-binding protein. Proteins are detected and quantified by the absorbance measured at 280 nm.

FIG. 4 is a picture of a reducing sodium dodecyl sulfate-polyacrylamide gel electrophoresis (SDS-PAGE) gel loaded with samples obtained following PSP94-affinity chromatography. The gel was run in an electric field and stained with Gelcode® Blue Code Reagent (Pierce). Lane 1 represents the molecular weight marker. Lane 2 represents proteins bound to the PSP94-conjugated affinity matrix. Lane 3 represents proteins that bound to the PSP94-conjugated affinity matrix when excess free PSP94 was included within the incubation mixture.

FIG. 5 is a picture of a non-reducing SDS-PAGE gel loaded with samples obtained following the elution of the PSP94-binding protein from the PSP94-conjugated affinity matrix using different eluting (dissociation) conditions. After incubation, in the different eluting buffers, the affinity matrix was removed from the eluting buffer by centrifugation. The matrix was washed in PBS, and boiled in non-reducing SDS-PAGE sample buffer. The SDS-PAGE was run in an electric field and was stained with Gelcode® Blue Code Reagent (Pierce). Arrows represent the position of the high molecular weight binding protein (HMW) and the low molecular weight binding protein (LMW). Lane A represents the molecular weight marker. Lane B represents untreated sample. Lane C represents sample incubated for 1 hour in PBS at 34 ²C. Lane D represents sample incubated for 1 hour in water at 34° C. Lane E represents sample incubated with 300 μg of PSP94 in 1 ml of PBS at 34° C. Lane F represents the competition control. Lane G represents sample incubated in 2 M urea. Lane H represents sample incubated in 8 M urea. Lane I represents sample incubated in 100 mM sodium acetate at pH 2.7. Lane J represents sample incubated in 100 mM 3-(Cyclohexylamino)-1-propanesulfonic acid (CAPS) at pH 11.0.

FIG. 6 is a graph showing affinity chromatography (using PSP94-conjugated affinity matrix) results of samples purified by ammonium sulfate precipitation followed by anion-exchange chromatography. PSP94-binding protein was eluted from the column by adding excess PSP94. The peak located between point A and B represents the PSP94-binding protein fraction. Proteins are detected and quantified by the absorbance at 280 nm.

FIG. 7 is a picture of a SDS-PAGE performed in non-reducing conditions. Lane A is the molecular weight marker. Lane B represents the PSP94-affinity matrix after incubation with PSP94-binding protein purified by ammonium sulfate precipitation and anion-exchange chromatography, and prior to elution with competing PSP94. Lane C represents the competition control. Lane D represents the affinity matrix after elution with excess PSP94. Lane E represents the final eluted and concentrated (substantially) pure PSP94-binding protein.

FIG. 8 is a schematic of a proposed purification process for the PSP94-binding protein.

FIG. 9a is a picture of a Northern blot performed on samples of human tissue poly-A RNA. Lane 1 represents brain RNA, lane 2 represents heart RNA, lane 3 represents skeletal muscle RNA, lane 4 represents colon RNA, lane 5 represents thymus RNA, lane 6 represents spleen RNA, lane 7 represents kidney RNA, lane 8 represents liver RNA, lane 9 represents small intestine RNA, lane 10 represents placenta RNA, lane 11 represents lung RNA and lane 12 represents peripheral blood lymphocytes (PBL) RNA.

FIG. 9b is a picture of a Northern blot performed on samples of human tissue poly-A RNA. Lane 1 represents spleen RNA, lane 2 represents thymus RNA, lane 3 represents prostate RNA, lane 4 represents testis RNA, lane 5 represents ovary RNA, lane 6 represents small intestine RNA, lane 7 represents colon RNA and lane 8 represents peripheral Blood Lymphocytes (PBL) RNA.

FIG. 10 is a picture of a Western blot showing recognition (binding) of PSP94-binding protein with a specific monoclonal antibody (1B11) Lane 1 is molecular weight markers (from top to bottom, 212, 132, 86, 44 kDa). Lane 2 is 0.2 μg of (substantially) purified PSP94-binding protein and lane 3 is 25 μl of partially pure PSP94-binding protein.

FIG. 11 is a picture of an ELISA plate where the specificity of monoclonal antibodies for bound and free forms of PSP94 is evaluated. Colored wells represent a positive result.

FIG. 12a is a schematic of a method used to measure the amount of free PSP94. FIG. 12b is a result of an ELISA assay using the method illustrated in FIG. 12a.

FIG. 13 is a schematic of a proposed method used to measure the amount (PSP94 sandwich ELISA) of total PSP94 in a sample.

FIG. 14a is a schematic of a method used to measure the amount of total PSP94-binding protein (using a PSP94-binding protein sandwich ELISA) in a sample. FIG. 14b is a result of an ELISA assay used to measure the PSP94-binding protein in a sample using the method illustrated in FIG. 14a.

FIG. 15A represents concentration of total PSP94 levels from serum of individuals in low (<4 ng/ml) and high (>4 ng/ml) PSA categories.

FIG. 15B represents concentration of free PSP94 levels from serum of individuals in low (<4 ng/ml) and high (>4 ng/ml) PSA categories.

FIG. 15C represents concentration of total PSP94 Binding protein levels from serum of individuals in low (<4 ng/ml) and high (>4 ng/ml) PSA categories.

FIG. 15D represents concentration of corrected free PSP94 levels from serum of individuals in low (<4 ng/ml) and high (>4 ng/ml) PSA categories. Free PSP94 values were corrected since 1-5% of PSP94 binding protein (and complexed PSP94) remained after absorption protocol. The correction subtracts the bound PSP94 x proportion of PSP94 binding protein not absorbed from the uncorrected free PSP94 value.

FIG. 16 represents total PSP94 binding protein concentration compared to total PSP94.

### DETAILED DESCRIPTION OF THE INVENTION

PSP94 was used as a bait in the isolation and identification of a PSP94-binding protein. For that purpose, labeled-PSP94 was used to detect the presence of the PSP94-binding protein(s) in serum fractions that were submitted to various purification steps. In addition, PSP94 was used for affinity chromatography purification of the PSP94-binding protein. Examples described below illustrate the purification, identification and utility of the PSP94-binding protein.

### EXAMPLE 1

Radiolabeling of PSP94 and PSP94-Binding Protein Kinetic Analysis.

Experiments to optimize ¹²⁵I-PSP94 labeling, ¹²⁵I-PSP94 binding assay to human male serum proteins and development of means to separate free-(i.e., unbound) and complexed-(i.e., bound, associated) ¹²⁵I-PSP94 were undertaken. Human male serum protein(s) that will bind to PSP94 (in the present case; ¹²⁵I-PSP94) will generate the formation of a complex of higher molecular weight than free-PSP94 (or free 125I PSP94).

Iodination of PSP94 was performed as followed. Twenty micrograms of native human PSP94 prepared as previously described (Baijal Gupta et al., Prot. Exp. and Purification 8:483-488, 1996) in 15 microliters of 100 mM sodium bicarbonate (pH 8.0) was labeled using one millicurie of mono-iodinated Bolton-Hunter reagent at 0° C. following the manufacturer's instructions (NEN Radiochemicals). The reaction was terminated after 2 hours by the addition of 100 microliters of 100 mM glycine. The free iodine was separated from the iodine incorporated into the PSP94 by a PD10 disposable gel filtration column according to manufacturer's instructions (BIORAD). Typically, the proportion of iodine that became incorporated into the PSP94 protein was about 60%, giving a specific activity of about 30 microcuries per microgram of PSP94.

Optimization of the binding assay of human male serum proteins to ¹²⁵I-PSP94 was performed in order to identify the optimal incubation time, temperature, and separation conditions. Equilibrium (e.g., no further significant increase in binding as incubation time lengthens) was approached after a considerable incubation time at 37° C., so a 16 hours incubation time was selected. Separation of the complexed form (i.e., bound form) PSP94 (or complexed-1251-PSP94), having a higher molecular weight and the free-PSP94 (or free-1251-PSP94), having a low molecular weight, was effected by gel filtration chromatography, using Sephadex G100 resin (Amersham Pharmacia Biotech Ltd) packed into a 1×20 cm column. The molecular sieve chromatography was performed at 4° C. since at higher temperatures dissociation of the complex during the procedure was shown to be significant.

Based on the optimization results described above, radioligand binding analysis of PSP94-binding serum components (i.e., PSP94-binding protein) was performed. This assay was done in a total volume of 500 microliters. The test samples contained PSP94-binding protein (neat serum, or fractions from purification trials) 50 ng of radiolabeled PSP94, with or without excess free competitor (10 micrograms free PSP94 (unlabeled)) in phosphate buffered saline-gelatin (PBS-gelatin: 10 mM sodium phosphate, 140 mM NaCl, 0.1% gelatin (Fisher Scientific, Type A), pH 7.5, including 8 mM sodium azide as an antibacterial agent). Those were incubated for 16 hours at 37° C. At this time, the equilibrated mixture was placed on ice, and the components separated according to their molecular weight by molecular sieve chromatography at 4° C. using a 1×20 cm sephadex G100 column equilibrated with PBS-gelatin. After the sample had run into the column, 3 ml was discarded, and 20 fractions of 0.5 ml were collected. A single fraction of 30 ml was also collected at the end of the run.

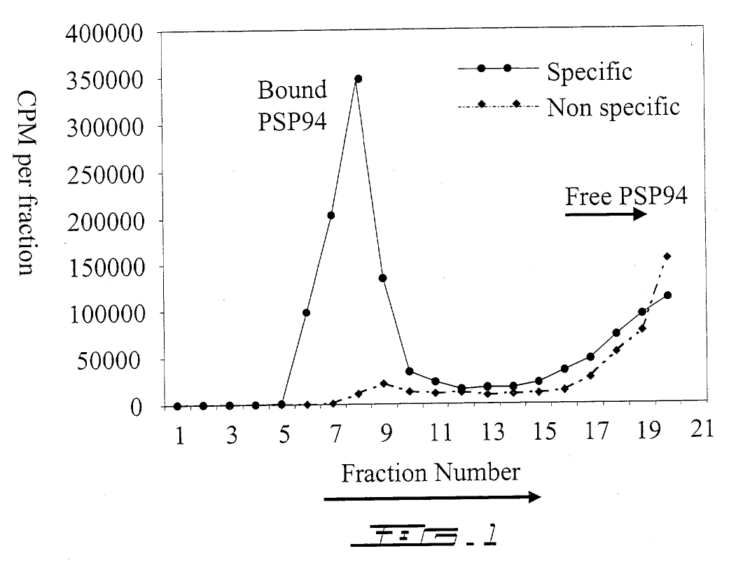

The radioactivity (expressed in counts per minute (cpm)) in the collected fractions was measured using an LKB rack gamma counter, and the total radioactivity in the high molecular weight peak (generally contained within fractions 4-14) and low molecular weight peak (the remainder of the 0.5 ml fractions and the single 30 ml fraction) were calculated. A typical elution profile is illustrated in FIG. 1.

FIG. 1 shows size exclusion chromatography results of proteins from human male serum bound to PSP94 radiolabeled with isotope 125 of iodine (¹²⁵I) (i.e., ¹²⁵I-PSP94) (specific biding). Binding of ¹²⁵I-PSP94 to human male serum protein is determined by the radioactivity, expressed in counts per minute (cpm), in each fraction. Non-specific binding was determined by including 10 μg of free PSP94 in the incubation mixture together with 250 μl of human male serum and 50 ng of ¹²⁵I-PSP94. The location of fractions containing free-(i.e., unbound) and complexed (i.e., bound)-PSP94 are indicated in the graph. The majority of the free PSP94 (¹²⁵I-PSP94) eluted later than fraction 20. Typically, about 33% of the total radioactive PSP94 added to the 250 microliters of human serum eluted in the earlier fractions as part of the PSP94-binding protein complex, and about 67% of the radioactive PSP94 remained uncomplexed eluting in the later fractions. In the competitive control, with the inclusion of 10 micrograms of unlabelled PSP94 in the incubation mixture, only about 3% of the radioactive PSP94 eluted in the earlier fractions as part of a high molecular weight complex, confirming the specificity of the PSP94 for the PSP94-binding protein.

Using this methodology, and by varying the concentration of radiolabeled and competing PSP94 and maintaining the quantity of human male serum, constant (250 μl) it was possible to perform kinetic analysis of the equilibrium binding data. Assuming that PSP94 is about one fifth of the molecular weight of a PSP94-binding protein, this would suggest that each milliliter of serum has about 1 microgram of PSP94-binding protein. The total protein content of serum is about 80 milligrams per milliliter, so the PSP94-binding protein: total protein ratio in serum is approximately 1:80,000.

Further information from radioligand binding analysis indicated that a PSP94-binding protein is present in human female serum, virgin female human serum, fetal bovine serum, and pooled mouse serum.

### EXAMPLE 2

### Ammonium Sulfate Precipitation

From the kinetic results obtained in example 1, it was shown that the PSP94-binding protein was poorly abundant in human serum.

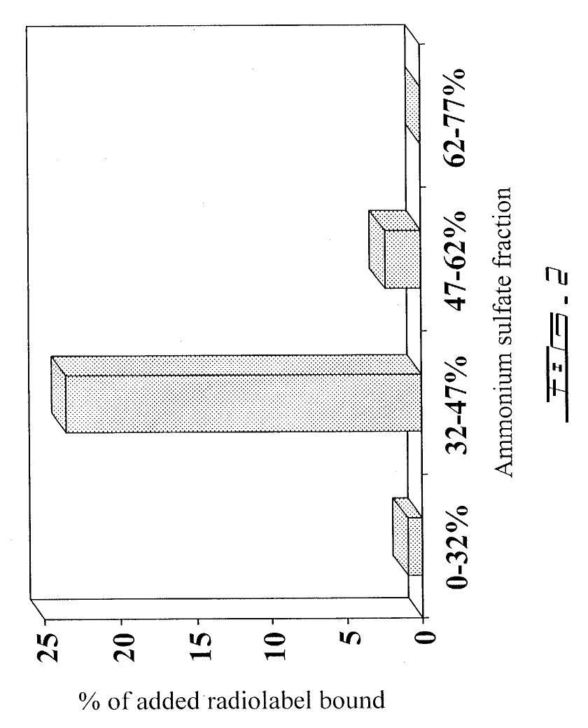

In order to isolate a PSP94-binding protein for further characterization and identification, a first purification step was performed by ammonium sulfate precipitation. To establish the appropriate concentration of ammonium sulfate necessary to precipitate a PSP94-binding protein, small scale ammonium sulfate precipitation trials were performed. The presence of a PSP94-binding protein in the precipitate was determined after dissolution and dialysis against PSP94 by radioligand binding analysis as discussed in example 1. These trials determined that the 32-47% ammonium sulfate fraction contained the vast majority of a PSP94 binding material as illustrated in FIG. 2.

Ammonium sulfate precipitation was routinely performed on a larger scale. Briefly, 1 liter of male frozen serum (Bioreclamation Inc, New York) was thawed and added to 1 liter of cold 10 mM Sodium Phosphate, 140 mM NaCl, pH 7.5 (phosphate buffered saline; PBS), and to this 370 g of ammonium sulfate (BDH ACS reagent grade) was added slowly under constant stirring to bring the ammonium sulfate concentration up to 32%. After dissolution of the salt, the mixture (i.e., male serum containing ammonium sulfate) was stirred for 20 minutes before centrifugation at 5,000×g for 15 minutes. The pellet was discarded, and the supernatant fraction of proteins containing a PSP94-binding protein was collected. Further ammonium sulfate (188 g) was added slowly under constant stirring to the supernatant fraction, bringing the total ammonium sulfate concentration to 47%. After 20 minutes, this mixture was also spun at 5,000×g, the supernatant was discarded, and the pellet was dissolved in a total of 500 ml of 10 mM MES ((2-[N-Morpholino]ethanesulfonic acid) hydrate), 100 mM NaCl, pH 6.5. This pellet was dialyzed using 6-8,000 molecular weight cut off dialysis tubing (Spectra/Por, Fisher Scientific Canada) with 16 liters of 10 mM MES, 100 mM NaCl, pH 6.5 for 16 hours at 4° C. followed by another dialysis step using a further 16 liters of the same buffer for an additional 7 hours. The protein concentration within the product was measured using 280 nm ultraviolet (UV) absorbance and the preparation was stored at −20° C. in 4 g of protein aliquots (generally about 150 ml). A typical ammonium sulfate precipitation assay is shown in FIG. 2.

### EXAMPLE 3

### Ion-Exchange Chromatography Assays

Ion exchange chromatography (IEX) separates molecules based on their net charge. Negatively or positively charged functional groups are covalently bound to a solid support matrix yielding a cation or anion exchanger. When a charged molecule is applied to an exchanger of opposite charge it is adsorbed, while neutral ions or ions of the same charge are eluted in the void volume of the column. The binding of the charged molecules is reversible, and adsorbed molecules are commonly eluted with a salt or pH gradient.

Without prior knowledge of any characteristics of the PSP94-binding protein, the ability of anion and cation exchange matrices to absorb a PSP94-binding protein at a range of pH values was determined in a series of ion-exchange assays. Aliquots of ammonium sulfate precipitated serum were exchanged into the buffers indicated in table 3 using a Biorad DG 10 column equilibrated with the appropriate buffer according to the manufacturer's instructions. Seven hundred microliters aliquots were incubated with 500 microliters of ion-exchange matrix (prepared according to the manufacturer's recommendations). After incubation for 90 minutes at room temperature with gentle agitation, the mixture was spun at 1000×g for 5 minutes to separate the matrix from the supernatant. If a PSP94-binding protein is bound (adsorbed) to the matrix, it will remain bound to it after centrifugation and will not be present in the supernatant. The supernatant was immediately neutralized with 0.3 volumes of 250 mM TRIS pH 7.5 and 250 microliters of this solution was assessed in the ¹²⁵I-PSP94 binding assay described herein (example 1). Conditions tested and results of these assays are presented in table 3.

TABLE 3

| | | ¹²⁵I-PSP94 | ¹²⁵I-PSP94 |
|---|---|---|---|
| | | binding before | binding after |
| | | incubation with | incubation with |
| | Buffer | matrix | matrix |
| Cation Matrix: | | | |
| Macro Prep High S | | | |
| (BIORAD) | | | |
| pH 4.7 | 10 mM Citrate | 9.5% | 0.08% |
| pH 5.7 | 10 mM MES | 11.9% | 7.7% |
| pH 6.7 | 10 mM MES | 20.6% | 18.6% |
| pH 7.9 | 10 mM MOPS | 20.5% | 11.9% |
| Anion Matrix | | | |
| Macro Prep High Q | | | |
| (BIORAD) | | | |
| pH 5.7 | 10 mM MES | 11.9% | 0.73% |
| pH 6.7 | 10 mM MES | 20.6% | 0.66% |
| pH 8.0 | 10 mM Bicine | 14.1% | 0.81% |
| pH 9.0 | 10 mM Bicine | 12.5% | 0.65% |

The major findings from these ion-exchange chromatography assays indicate that temporary exposure of a PSP94-binding protein to extremes of pH (8 and above, and 6 and below) resulted in a reduced ability of a PSP94-binding protein to bind to PSP94, suggesting that a PSP94-binding protein is pH sensitive. No adsorption of PSP94-binding protein to the cation matrix was seen at pH 4.7. Some adsorption to the cation matrix was seen at pH 5.7 and maximal adsorption was seen at pH 6.7. These results may suggest an isoelectric point of about pH 5.

The anion-exchange chromatography assays indicated good adsorption of a PSP94-binding protein to the matrix between pH 5.7 and 9.0, consistent with an isoelectric point of 5. It was clear that a preferred purification strategy would have to use the anion-matrix, because good adsorption could be attained at neutral (non-denaturing) pH values. So the anion-exchange matrix, and the 10 mM MES buffer at pH 6.5 was selected for further work using salt concentration elution rather than pH elution.

Optimization of conditions of PSP94-binding protein elution from the anion-exchange matrix was performed using various sodium chloride concentration.

A column (1×15 cm) containing Macro Prep High Q was equilibrated with buffer containing 10 mM MES, 100 mM NaCl, pH 6.5 and run at 0.5 ml per minute. Seven milliliters of the 32-47% ammonium sulfate cut (i.e., starting material of table 4) equilibrated into the same buffer, was applied to the column, and various buffers were applied to elute a PSP94-binding protein. The eluant was monitored with a UV recorder. The fractions were collected, and buffer was exchanged into PBS using CentriPrep concentrators with a molecular weight cut off of 10 kDa (Amicon). These samples were tested in the ¹²⁵I-PSP94 binding assay described in example 1. Table 4 summarizes the different conditions used and the results obtained in this experiment. A star (*) indicate that some losses was experienced during buffer exchange. Protein concentrations were estimated from absorbance at 280 nm (A280) with 1 OD unit equivalent to 1 mg of protein.

TABLE 4

| Sodium chloride | Total protein | Total protein in | %¹²⁵I-PSP94 |
|---|---|---|---|
| concentration | Eluted (mg) | binding assay | bound |
| Starting | 179 mg* | 7.2 mg | 12.7% |
| material | | | |
| (ammonium | | | |
| sulfate cut) | | | |
| 100 mM | 50 mg | 0.67 mg | 0.89% |
| (flow through) | | | |
| 200 mM | 37 mg | 0.80 mg | 1.4% |
| 300 mM | 12 mg | 0.63 mg | 24.4% |
| 400 mM | 5 mg | 0.30 mg | 1.5% |
| 500 mM | 8 mg | 0.62 mg | 0.9% |
| 1000 mM | 7 mg | — | — |

From these data, it is clear that the buffer containing 300 mM NaCl was effective and would be preferably used for eluting a PSP94-binding protein from the anion-exchange matrix. Using these results, a scale up ion-exchange protocol was developed allowing the application of 4 g of ammonium sulfate precipitated serum extract to a 5 cm×12 cm anion-exchange matrix as described below.

### EXAMPLE 4

### Large-Scale Anion-Exchange Chromatography Purification of PSP94-Binding Protein

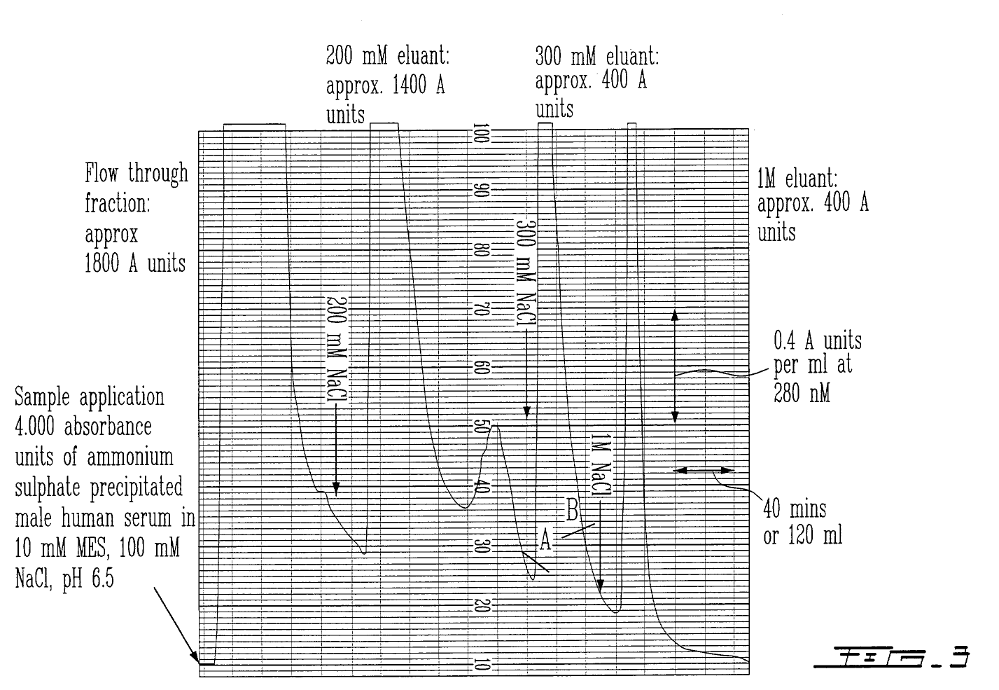

An anion exchange column (5 cm diameter x 12 cm length, Macro-Prep Hi Q, Biorad) was prepared and equilibrated in accordance with the manufacturer's guidelines in 10 mM MES, 100 mM NaCl, pH 6.5 and run at room temperature with a flow rate of about 3 ml per minute. An aliquot of ammonium sulfate precipitated serum (from example 2; 4 g total protein in about 150 ml of solution) was applied to the column which, was then washed with about 250 ml of 10 mM MES, 100 mM NaCl, pH 6.5 (FIG. 3). Elution was performed with about 400 ml of 10 mM MES, 200 mM NaCl, pH 6.5 buffer, followed by elution with 10 mM MES, 300 mM NaCl. The 300 mM eluting fraction was collected (FIG. 3). The profile of the eluting proteins was monitored by UV absorbance at 280 nm on a chart recorder. A typical profile is illustrated in FIG. 3. FIG. 3 is a graph showing anion-exchange chromatography results using a MacroPrep High Q anion exchange column, loaded with proteins purified by ammonium sulfate (about 4 grams). Proteins are eluted with stepwise increases in sodium chloride concentration. The peak located between point A and B represents the protein fraction containing a PSP94-binding protein. Proteins are detected by the absorbance measured at 280 nm.

The column could be regenerated with 10 mM MES, 1 M NaCl, pH 6.5 (300 ml) followed by an equilibration with 500 ml of 10 mM MES, 100 mM NaCl, pH 6.5. Sodium azide was added to this buffer at 0.05% (w/v) for storage of the column for greater than 24 hours.

The 300 mM fraction (about 90 ml) was collected (between markers A and B, FIG. 3) and this was shown previously to contain the majority of a PSP94-binding activity. This preparation identified “partially pure PSP94-binding protein” (PPBP) was concentrated to about 20 ml in centrifugal concentrators according to the manufacturer's instruction (Centriprep 10, Amicon) diluted with PBS to 60 ml, concentrated to 20 ml, further diluted with PBS to 60 ml, concentrated to 20 ml, and finally diluted with PBS to give a solution with an A280 of 2.0 (generally a final volume of about 150 ml). This solution was stored at −20° C. After a total application of 20 g of protein (5 cycles) the column was sanitized using 1 M NaOH and re-equilibrated in 10 mM MES, 100 mM NaCl, pH 6.5 using the protocol described by BIORAD.

Ammonium sulfate fractionation (i.e., precipitation) and anion exchange chromatography have resulted in approximately 4 fold and 10 fold purification of a PSP94-binding protein respectively. In neat serum, estimations indicated that the ratio of PSP94-binding protein total protein was 1:80,000. The efficiency of the two protein purification steps described in example 2 and example 4 were monitored using the PSP94 radioligand binding assay described in example 1. In both steps, the vast majority of the PSP94 binding material was confined within a single fraction. From this information, it appears that in combination, these two steps result in an efficient purification process with little loss (qualitatively) of the PSP94 binding material. However, assuming losses are small, the partially purified binding protein (PPBP) yielded by the combination of the two protein purification steps described in examples 2 and 4, should contain about 1 part of binding protein: 2000 parts of other proteins, by mass.

### EXAMPLE 5

### Affinity Chromatography Assays

Preparation of affinity matrix for PSP94-binding protein purification was performed as followed. Approximately 0.5 g of cyanogen bromide activated sepharose CL 4B (Sigma Chemical Company) was swelled in 1 mM HCl and prepared as per the manufacturer's recommendations. To 1 ml of this matrix, 5 ml of a solution containing 5 mg of PSP94 purified as described in Baijal Gupta et al. (Prot. Exp. and Purification 8:483-488, 1996) in 100 mM NaHCO₃ 0.5 M NaCl, pH 8.0 was added and the reactants incubated at 4° C. with periodic agitation. At time intervals, the reactants were spun at 200×g for 2 minutes, and the absorbance at 280 nm (A280) expressed in optical density (OD) units, of an aliquot of supernatant was measured in order to determine the proportion of binding of PSP94 to the matrix. Results showing the time course of conjugation (i.e., binding) of PSP94 to the activated sepharose (i.e., matrix) are summarized in table 5.

TABLE 5

| Duration of | A280 (OD) units | | |
|---|---|---|---|
| reaction | not bound to | A280 (OD) units | % of PSP94 |
| (min) | matrix | bound to matrix | incorporation |
| 0 (start) | 5.1 | 0 | 0 |
| 5 | 4.7 | 0.48 | 9.6 |
| 15 | 3.0 | 2.1 | 41 |
| 30 | 2.0 | 3.1 | 61 |
| 60 | 1.6 | 3.5 | 69 |

The conjugation reaction was continued until 70-80% of the PSP94 had bound to the matrix (after about 60 minutes in the preparation illustrated in table 5). At this time, 1 ml of 200 mM glycine was added to block any further reactive groups and the slurry was incubated overnight at 4° C. with gentle agitation. The matrix was washed according to the manufacturer's recommendations and diluted in PBS to give a slurry with a concentration with respect to PSP94 of 1 microgram per microliter. Sodium azide (NaN₃) was added to 0.05% as an anti-microbial agent.

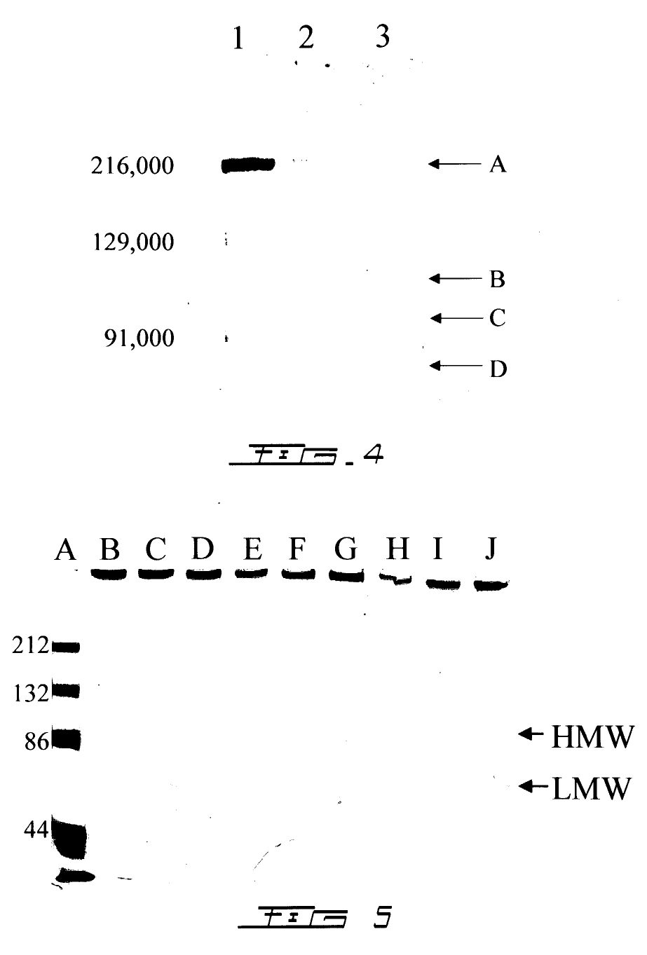

Based on the results of optimization assay described above, a PSP94 affinity matrix was prepared by conjugating PSP94 to cyanogen bromide activated sepharose. The matrix typically had 4 micrograms of PSP94 per microliter of packed matrix, and a working slurry with 1 microgram of PSP94 per microliter was prepared by dilution with PBS containing 0.05% NaN₃. The PSP94 affinity matrix (at a concentration of 5 micrograms per milliliter with respect to PSP94) was added to the partially pure PSP94-binding protein. Tween 20 at a concentration of 0.1% (v/v) and NaN₃ at 0.05% (w/v) were also included in the mixture, which was then incubated at 34 ²C for 18 hours on a rocking table. In a parallel control experiment, free-PSP94 was also added at a concentration of 50 micrograms per milliliter. The addition of free PSP94 in this control experiment would compete with the PSP94 conjugated to the matrix for the binding of a PSP94-binding protein. This will reverse the binding of a PSP94-binding protein to the affinity column thus enabling the identification of proteins specifically binding to PSP94. The affinity matrix was separated from the supernatant by rapid filtration, and the matrix was extensively washed in PBS at 4° C. The matrix was collected and boiled in sodium dodecyl sulfate-polyacrylamide gel electrophoresis (SDS-PAGE) reducing sample buffer (final concentration in sample: 5 mM Tris pH 6.8, 2% (w/v) SDS, 10% glycerol (v/v), 8 mM dithiothreitol, 0.001% Bromophenol blue) to dissociate the bound proteins and these were resolved by 7.5% SDS-PAGE. Result of this experiment is illustrated in FIG. 4

FIG. 4 shows results of a sodium dodecyl sulfate-polyacrylamide gel electrophoresis (SDS-PAGE) loaded with samples obtained following PSP94-affinity chromatography. The gel was run in an electric field and stained with Coomassie Brilliant Blue. Lane 1 represents the molecular weight marker (Kaleidoscope prestained standards, Bio-Rad). Lane 2 represents proteins bound to the PSP94-conjugated affinity matrix. Lane 3 represents proteins bound to PSP94-conjugated affinity matrix and incubated with excess of PSP94. Note that at least two proteins, A and C, remain present in the two lanes, (lane 2 and 3). Two bands, B and D, are present in the lane 3 but not in the control experiment (lane 2). These bands (B and D) are likely to be specific PSP94-binding proteins.

### EXAMPLE 6

### Optimization of PSP94-Binding Protein Elution From the PSP94-Affinity Matrix

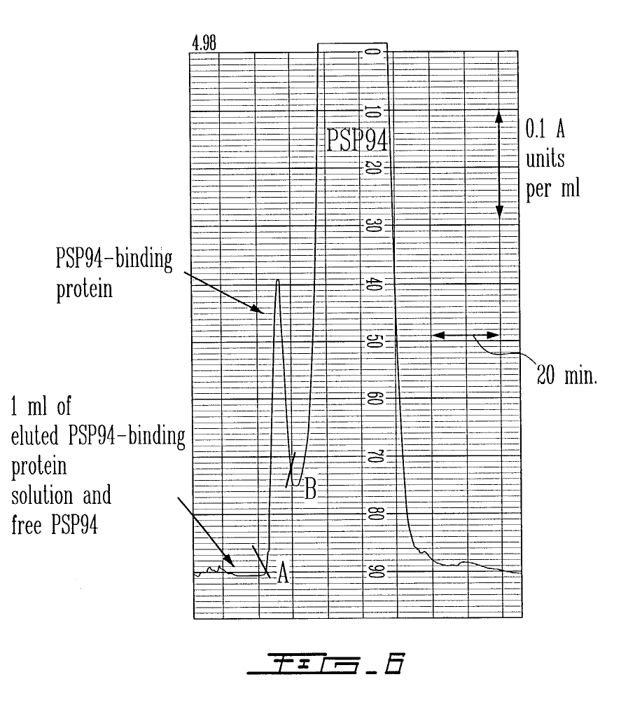

A range of conditions were assessed in order to dissociate a PSP94-binding protein from the affinity matrix using less denaturing conditions than boiling in SDS-PAGE sample buffer (either in non-reducing conditions or not). Conditions tested are summarized in table 6. Undenatured active PSP94-binding protein is required for antibody generation and further experimentation and development. Aliquots of PSP94-affinity matrix that had been pre-incubated with partially pure PSP94-binding protein and washed (i.e., with binding protein attached) were incubated for 1 hour in the elution (dissociation) conditions listed in table 6. After incubation, the affinity matrices were removed from the eluting buffers by centrifugation. The matrices were washed in PBS, and boiled in non-reducing SDS-PAGE sample buffer (final concentration in sample: 5 mM Tris pH 6.8, 2% (w/v) SDS, 10% glycerol (v/v), 0.001% Bromophenol blue) and proteins were resolved on 7.5% SDS-PAGE. If proteins remains associated with the matrix after elution, the conditions are not suitable for an appropriate dissociation. Thus if a PSP94-binding protein is absent from the SDS-PAGE illustrated in FIG. 5, elution (dissociation) conditions are suitable. Non-reducing conditions were found to provide superior separation conditions, because the major contaminating band was left at the top of the gel, rather than between the two PSP94-binding protein bands. Conditions tested and results of this experiment are illustrated in FIG. 5 and summarized in table 6.

TABLE 6

| | | Effect on PSP94-binding |
|---|---|---|
| Lane | Dissociation conditions | protein |
| A | Molecular weight marker | — |
| B | No treatment | None |
| C | 1 hour in PBS at 34° C. | None observable |
| D | 1 hour in water at 34° C. | None observable |
| E | 300 μg PSP94 in 1 ml PBS at 34° C. | Near total elution from |
| | | matrix |
| F | (Competition control) | (near full competition) |
| G | 2 M urea | None observable |
| H | 8 M urea | Some loss of binding |
| I | 100 mM sodium acetate pH 2.7 | Some loss of binding |
| J | 100 mM CAPS pH 11.0 | Some loss of binding |

FIG. 5 shows a SDS-PAGE loaded with samples obtained following the elution of a PSP94-binding protein from the PSP94-conjugated affinity matrix using different eluting (dissociation) conditions. After incubation, in the different eluting buffers, the affinity matrix was removed from the eluting buffer by centrifugation. The matrix was washed in PBS, and boiled in non-reducing SDS-PAGE sample buffer. The SDS-PAGE was run in an electric field and was stained with Gelcode® Blue Code Reagent (Pierce). Arrows represent the position of the high molecular weight binding protein (HMW) and the low molecular weight binding protein (LMW). Lane A represents the molecular weight marker (Kaleidoscope prestained standards, Bio-Rad). Lane B represents untreated sample. Lane C represents sample incubated for 1 hour in PBS at 34° C. Lane D represents sample incubated for 1 hour in water at 34° C. Lane E represents sample incubated with 300 μg of PSP94 in 1 ml of PBS at 34° C. Lane F represents the competition control, where the matrix was incubated with the PPBP in the same way as the sample from lane B, but included in this incubation was a saturating excess of free PSP94. Lane G represents sample incubated in 2 M urea. Lane H represents sample incubated in 8 M urea. Lane I represents sample incubated in 100 mM sodium acetate at pH 2.7. Lane J represents sample incubated in 100 mM 3-(Cyclohexylamino)-1-propanesulfonic acid (CAPS) at pH 11.0.

From the experiment described above, it is clear that a PSP94-binding protein and PSP94-affinity matrix interaction was highly stable under a variety of conditions. Some dissociation was seen with 8 M urea, and extremes of pH, however these denaturing conditions were less favored than non-denaturing competitive dissociation using excess free ligand (i.e., PSP94). This approach was therefore selected in order to purify the active PSP94-binding protein.

Data indicate that the HMW and LMW bands of FIG. 5 are the same as bands B and D of FIG. 4, respectively.

### EXAMPLE 7

### PSP94-Binding Protein Purification by PSP94-Affinity Chromatography

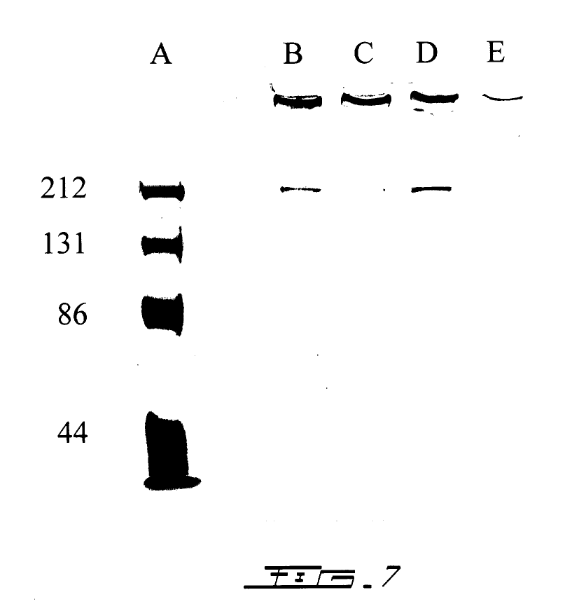

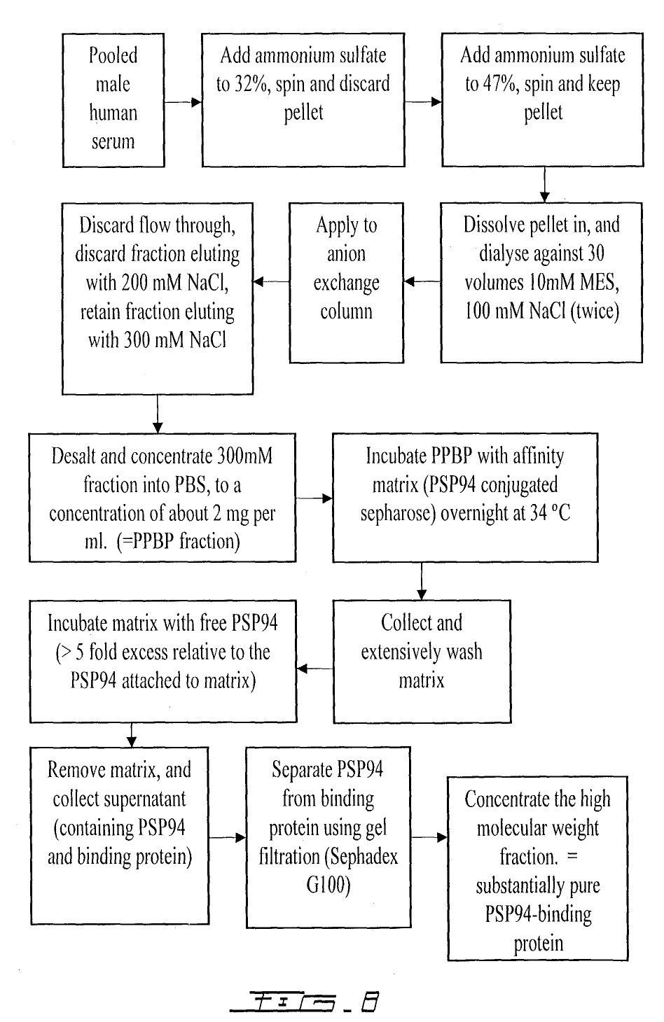

One hundred milliliters of partially pure PSP94-binding protein (preparation generated as described in-example 4), containing 0.1% (v/v) Tween-20 and 0.05% (w/v) NaN₃, was incubated with 250 micrograms (with respect to PSP94) of affinity matrix for 16 hours at 34° C. The matrix was separated from the soluble fraction by rapid filtration using a disposable Poly-Prep Column (Bio Rad). The liquid was forced through the column by applying air pressure from a 10 ml syringe attached to the column end cap. The matrix was washed three times with 10 ml of ice cold PBS similarly, and the matrix was collected from the column's polymer bed support with a micropipette. The matrix was resuspended in 1 milliliter of 10 mM sodium phosphate, 500 mM NaCl pH 7.5 containing 2 mg of free PSP94 and incubated with gentle agitation for 5 hours at 34° C. The matrix was then separated from the solution by centrifugation (1000×g for 30 seconds) and the supernatant (containing the eluted PSP94-binding protein and free PSP94) was resolved by molecular sieve chromatography at room temperature using a 1×20 cm sephadex G100 column equilibrated with 10 mM sodium phosphate, 500 mM NaCl, pH 7.5 and run at a flow rate of approximately 0.7 ml per minute. The absorbance at 280 nm of the eluant was recorded on a chart recorder (FIG. 6). Qualitative assessments of PSP94-binding protein capture, elution, and purified product were made by non-reducing 7.5% SDS-PAGE (FIG. 7).

FIG. 6 shows affinity chromatography (using PSP94-conjugated affinity matrix (Sephadex G-100)) results of samples purified by ammonium sulfate precipitation and anion-exchange chromatography. PSP94-binding protein was eluted from the column by adding excess PSP94 (free-PSP94). The high molecular weight proteins were collected (between points A and B) in a total volume of 4 ml. This solution was buffer exchanged into PBS (150 mM NaCl) using centrifugal concentrators (Centricon-10 from Amicon) and concentrated to approximately 100 ng per microliter. Typical yield=40 micrograms from 100 ml of PPBP starting material. The peak located between points A and B represents a PSP94-binding protein fraction. Proteins are detected and quantified by the absorbance measured at 280 nm. Results obtained indicate a proper separation between free PSP94 and a PSP94-binding protein.

FIG. 7 is a picture of a SDS-PAGE (7.5%) performed in non-reducing conditions. Lane A is the molecular weight marker (Kaleidoscope prestained standards, Bio-Rad). Lane B represents a PSP94-affinity matrix after incubation with a PSP94-binding protein purified by ammonium sulfate precipitation and anion-exchange chromatography, and prior to elution with competing (i.e., excess) PSP94 (i.e., free-PSP94). Lane C represents the competition control. Lane D represents the affinity matrix after elution with excess PSP94. Lane E represents the final eluted and concentrated (substantially) pure PSP94-binding protein. Results obtained indicate that affinity chromatography increase the purity of a PSP94-binding protein(s) in a significant manner.

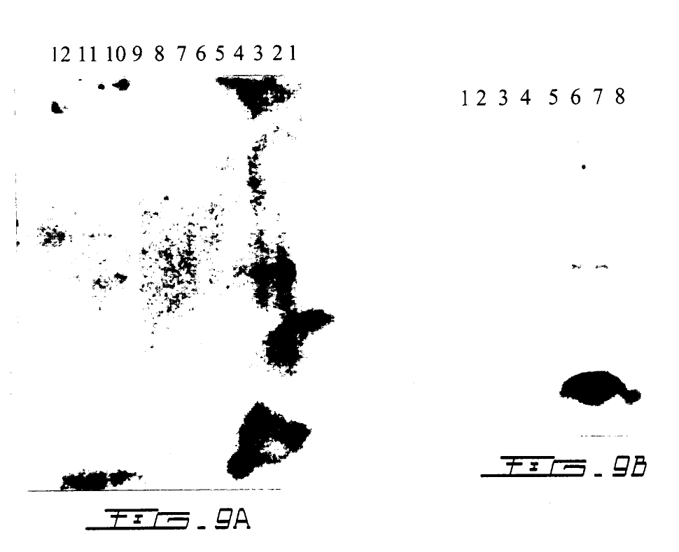

The purification process of a PSP94-binding protein has been summarized in FIG. 8.

### EXAMPLE 8

### PSP94-Binding Protein Amino-Terminal Amino Acid Sequencing

A SDS-PAGE gel was prepared as described in example 5. However the proteins were transferred to sequencing grade PVDF membranes (ProBlott membranes, Applied Biosystem) using a Mini Trans-Blot transfer cell (Bio-Rad) according to the manufacturer's recommendations for sequencing preparation. This membrane was stained with Coomassie Brilliant blue, and analyzed by amino-terminal (i.e., N-terminal) amino acid sequencing. The amino-terminal amino acid sequencing was carried out for bands B, C and D illustrated in FIG. 4.

TABLE 7

| Band | Amino acid Sequence |
|---|---|
| B | (L)TDE(E)KRLMVELHN |
| C | Ubiquitous immunoglobulin sequence |
| D | LTDEEKRLMVELHNLYRAQVSPTASDMLHM |

As seen in table 7 bands B and D have the same N-terminal amino acid sequences, so these are likely to be different forms of the same protein, with B possibly representing some form of aggregate (multi-mere), or alternatively, B and D being alternatively spliced, or processed.

### EXAMPLE 9

### Cloning of a PSP94-Binding Protein Gene Sequences

Total RNA was isolated from 2×10⁶ Jurkat clone E6-1 cells (TIB 152, American Type Culture Collection, Manassas, Va.) or from healthy blood donor peripheral blood mononuclear cells using Tri-reagent (Molecular Research Center Inc., Cincinnati, Ohio). RNA was ethanol-precipitated and resuspended in water. RNA was reverse transcribed into cDNA using the Thermoscript RT-PCR System (Life Technologies, Rockville, Md.). The cDNA was subsequently amplified by polymerase chain reaction (PCR) using Platinum Taq DNA Polymerase High Fidelity (Life Technologies) using a 5′-primer (5′-ATGCACGGCTCCTGCAGTTTCCTGATGCTT-3′) and a 3′-primer (5′-GCCCACGCGTCGACTAGTAC(T)₁₇-3′) (Life Technologies 3′Race adapter primer, Life Technologies). The 5′-primer DNA sequence was based on PSP94-binding protein amino acid sequence and partial cDNA sequence published in Gene Bank database (National Institute of Health, U.S.A.) G. B. Accession No. AA311654 (EST182514 Jurkat T-cells VI Homo sapiens cDNA 5′ mRNA sequence). Amplified DNA was resolved by agarose gel electrophoresis, excised from the gel and concentrated using Qiagen II DNA extraction kit (Qiagen, Mississauga, ON, Canada). Purified DNA was ligated into pCR2.1 plasmid (Invitrogen, Carlsbad, Calif.) and used to transform E. coli, strain TOP10 (Invitrogen). Ampicillin-resistant colonies were screened for cDNA-positive inserts by restriction enzyme analysis and DNA sequence analysis.

Blasting of DNA sequence of PSP94-binding protein into Gene Bank has identified some DNA sequence of unknown utility such as, for example, Gene Bank accession numbers XM 094933 (PRI Feb. 6, 2002), BC022399 (PRI Feb. 4, 2002), NM 153370 (PRI Apr. 7, 2003), BC035634 (PRI Sep. 23, 2002), etc.

### EXAMPLE 10

### Tissue Expression of PSP94-Binding Protein Messenger RNA

A PSP94-binding protein messenger RNA (mRNA) was isolated and the size and relative expression level in human tissues was determined by Northern blot. Commercial Northern blots containing 1 or 2 micrograms of human tissue poly-A RNA per lane (Multiple Tissue Northern (MTN™) Blot, Clontech, Palo Alto, Calif.) were hybridized as per the manufacture's recommendations with a [³²P]-labeled PSP94-binding Protein cDNA probe which spanned PSP94-binding Protein cDNA sequences 346 to 745. The intensity of the band was quantified with an alpha imager 2000, model 22595. The relative intensity of the band was determined and given an arbitrary score ranging from + to +++. This scoring was based on the lowest detectable 2.0 kb signal band seen.

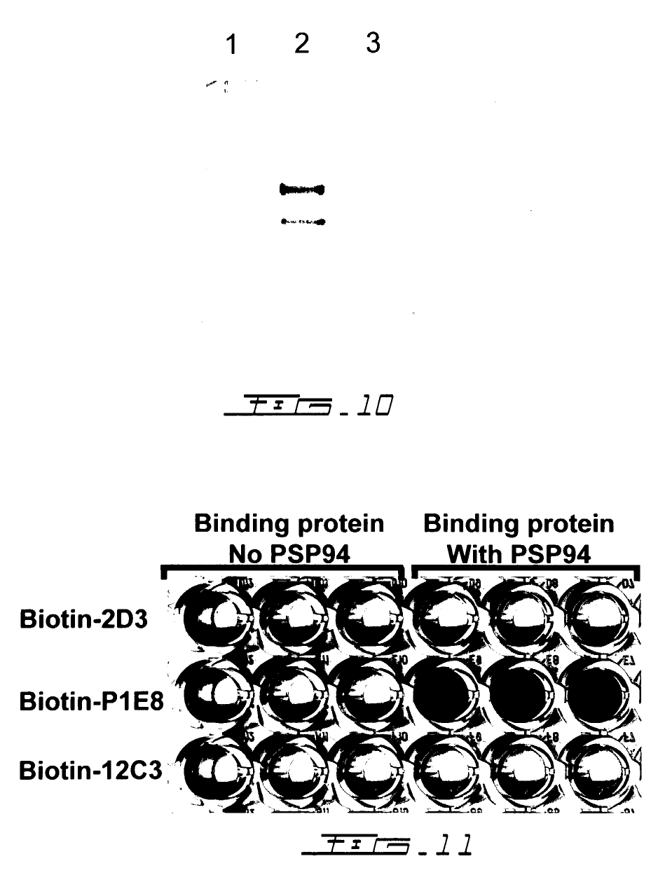

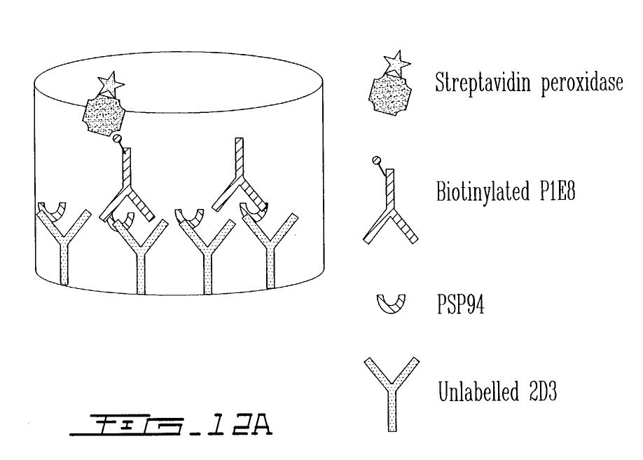

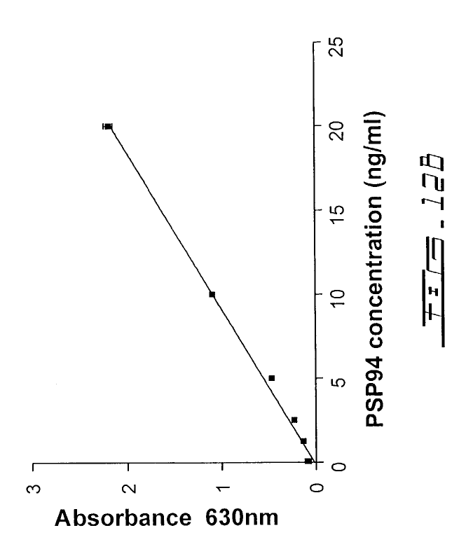

Quantification of the results illustrated in FIGS. 9a and 9b are summarized in tables 8 and 9 respectively. Briefly, RNA from brain, heart, skeletal muscle, colon, thymus, spleen, kidney, liver, small intestine, placenta, lung, prostate, testis, ovary, and peripheral blood lymphocytes (PBL) was analyzed for the expression of a PSP94-binding protein RNA expression.

TABLE 8

| Tissue | RNA signal (+) size kb | Relative intensity |
|---|---|---|
| Brain | 0 | |
| Heart | +2.0 | +++ |
| Skeletal muscle | +2.0 | ++ |
| Colon | +2.0 | + |
| Thymus | +2.0 | + |
| Spleen | | |
| Kidney | | |
| Liver | | |
| Small intestine | +2.0 | + |
| Placenta | | |
| Lung | | |
| Liver | | |

TABLE 9

| | RNA signal (+) and | |
|---|---|---|
| Tissue | size kb | Relative intensity |
| Spleen | | |
| Thymus | | |
| Prostate | +2.0 | +++ |
| Testis | +2.0 and 2.5 | ++ |
| Ovary | +2.0 | ++ |
| Small intestine | +2.0 | +++ |
| Colon | +2.0 | + |
| PBL | | |

### EXAMPLE 11

### Generation of Monoclonal Antibodies for Free PSP94, Bound PSP94 and PSP94-Binding Protein

Antibody Generation

The immunization scheme described herein was developed to promote the production of antibodies to epitopes of PSP94 that are exposed when bound to a PSP94-binding protein.

Four Balb/c mice (identified a, b, c and d) were immunized subcutaneously with 15 micrograms each of a (substantially) pure PSP94-binding protein (i.e., this preparation also contains PSP94) preparation in TiterMax™ adjuvant. Twenty-one days later, all mice were given a second boost and after a further 8 days, the mouse serum was tested for reactivity for both PSP94 and PSP94-binding protein in the ELISA screening assay described above. Since the purification of a PSP94-binding protein involves saturating all the binding sites with PSP94, the sera of the animals immunized with the substantially pure PSP94-binding protein preparation, tested positive for both antigens.

Mice a and b were boosted intra-peritoneally with a further 15 μg of a PSP94-binding protein with no adjuvant. The remaining two mice (c and d) were boosted subcutaneously with a further 15 μg of a PSP94-binding protein together with 15 μg of native PSP94 in Titer Max™ adjuvant in order to increase the likelihood of obtaining antibodies to exposed epitopes of PSP94.

After a further 4 days, the spleens of mice a and b were harvested, the B lymphocytes collected, and fused with NSO myeloma cells in order to generate hybridomas (Galfrè G. and Milstein C, Meth. Enzymol. 73:3-46, 1981). A hundred thousand splenocytes, in Iscove's MDM selection medium (supplemented with 20% FBS, HAT, 10 ng per ml interleukine-6, and antibiotics), were plated into each well of 96 well plates. Since antibodies are secreted from the cells, cell culture media (i.e., supernatant) may be harvested for characterization of the antibodies produced. After 10 days of incubation at 37° C., the supernatants of wells containing clones were assessed by an ELISA screening assay (see bellow). Clones producing antibodies showing a positive recognition (binding) of the PSP94 or PSP94-binding protein plates and free of unspecific binding to PBS coated plate, were selected for further investigation and characterization.

Desired (positive) clones were plated into 6 well plates. The supernatants were re-tested for the presence of the specific antibody, and those of the clones remaining positive were passed through successive cycles of cloning by limiting dilution. Cloning in such a manner insure that the hybridoma cell line produced is stable and pure. Typically, two cycles of cloning were necessary to achieve this goal. Multiple vials of frozen stocks were prepared, with one vial from each batch tested for viability and antibody production. Results of clone characterization are illustrated in table 10.

### EXAMPLE 12

### Antibody Characterization

ELISA-Based Hybridoma Screening Assay

In order to evaluate the titer and the specificity of the antibodies produced from mice or from the hybridoma generated from mouse B cells, an ELISA screening assay was developed.

Briefly, microtitre plates (Nunc, MaxiSorp) were coated with 100 μl aliquots of either native PSP94 (isolated from human seminal plasma; 5 μg/ml in 0.1 M sodium carbonate pH 9.6) or with a PSP94-binding protein (0.1 μg/ml in 0.1 M NaHCO₃) or phosphate buffered saline (PBS; 140 mM NaCl 10 mM sodium phosphate pH 7.5) overnight at 4° C. Plates were blocked for 1 hour with a solution of 1% bovine serum albumin (BSA) in phosphate buffered saline at 34° C. (BSA allows the saturation of the binding sites and limit unspecific binding to the plates). The plates (wells) were then washed in PBS containing 0.1% polyoxyethyylene-sorbitan monolaurate (PBS-Tween), prior to application of the mouse serum samples, or hybridoma supernatants diluted in 0.5% BSA. The plates were incubated for 1 hour at 34° C. prior to application of a 1:1000 dilution in PBS 0.5% BSA of peroxidase conjugated polyclonal rabbit immunoglobulins recognizing mouse immunoglobulins. (rabbit anti-mouse IgG peroxidase). After a further 1 hour incubation at 34° C. the plates were extensively washed in PBS Tween, prior to development of the peroxidase signal in 3,3′,5,5′-Tetramethylbenzidine (TMB). After 30 minutes the optical density at 630 nm was read in a micro plate reader.

Antibody Purification.

Mouse IgG1 monoclonal antibodies were purified using a high salt protein A procedure as detailed in Antibodies: A Laboratory Manual eds Harlow and Lane, Cold Spring Harbor Laboratory (for reference see above).

Antibody Isotyping

Isotyping was performed using a Mouse Monoclonal Antibody Isotyping Kit (Roche Diagnostics Corporation Indianapolis USA). This kit provides information relating to the class (IgG, IgA or IgM) the type of light chain (kappa or lambda) and IgG subtype (IgG1, IgG2a, IgG2b or IgG3). The antibodies tested were mainly of the IgG1 kappa subtype. However, one antibody was shown to be of the IgM kappa subtype (B26B10).

Relative Epitope Analysis

ELISA plates were coated either with a PSP94-binding protein or PSP94 and blocked as described above. Appropriate concentrations of the biotinylated antibodies prepared as described above were incubated with the coated plates in the presence or absence of a 50-fold excess of a panel of unlabelled antibodies. Competition with the unlabelled antibodies indicates epitopes that are shared between the two antibodies. Lack of competition indicates independent epitopes. Results of epitope analysis are illustrated in table 10.

TABLE 10

| | | Class and | Epitope shared | ATCC Patent |
|---|---|---|---|---|
| Clone | Specificity | subclass | with | Depository No. |
| 2B10 | Binding | IgG₁K | 9B6, 3F4 | — |
| | protein | | | |
| 1B11 | Binding | IgG₁K | Unique | — |
| | protein | | | |
| 9B6 | Binding | IgG₁K | 2B10, 3F4 | — |
| | protein | | | |
| 17G9 | Binding | IgG₁K | Unique | PTA-4243 |
| | protein | | | |
| 3F4 | Binding | IgG₁K | 2B10, 9B6 | PTA-4242 |
| | protein | | | |
| P8C2 | Binding | IgG₁K | Unique | — |
| | protein | | | |
| B3D1 | Binding | IgG₁K | — | — |
| | protein | | | |
| 26B10 | Binding | IgMK | — | — |
| | protein | | | |
| 2D3 | Free PSP94 | IgG₁K | Unique | PTA-4240 |
| P1E8 | Free and | IgG₁K | Unique | PTA-4241 |
| | bound (total) | | | |
| | PSP94 | | | |
| 12C3 | Free PSP94 | IgG₁K | Unique | — |

Antibody Biotinylation

The diluent (buffer) of the purified antibody was exchanged for 0.1 M NaHCO₃ buffer pH 8.0 and the protein concentration adjusted to 1 mg/ml. A 2 mg/ml solution of biotinamidocaproate N-hydroxysuccinimide ester was prepared in DMSO and an appropriate volume of this solution was added to the antibody to give either a 5, 10 or 20 fold excess of biotinylating agent. This was incubated on ice for 2 hours with occasional agitation before an equal volume of 0.2 M glycine in 0.1 M NaHCO₃ was added to give a final concentration of 0.1 M glycine.

After one further hour incubation on ice, the antibody was separated from the free biotinylating agent by gel filtration using a PD10 gel filtration column (Biorad). Biotinylated antibodies were stored at 4° C. in with 0.05% sodium azide added as preservative. The optimal extent of biotinylation and optimal usage concentration of the biotinylated antibodies was determined on antigen-coated plates.

Western Blots

Antibodies were assessed by Western blot. Briefly, 0.2 micrograms of (substantially) purified PSP94-binding protein, or 25 microliters of partially pure PSP94-binding protein were run on 7.5% SDS PAGE gels under non-reducing conditions. The proteins were transferred to PVDF membranes, the membranes were blocked with 1% BSA, probed with the hybridoma supernatants at a dilution of 1:5 (in PBS/0.5% BSA), and the bound antibody was detected with an anti-mouse immunoglobulin peroxidase-conjugate raised in rabbit. The signal was developed in 0.05% diaminobenzidine 0.01% hydrogen peroxide.

Specificity of PSP94 Antibodies for Free or Total PSP94

In order to further characterize the specificity of the antibodies generated herein, an assay was developed to determine if the monoclonal antibodies recognize PSP94 in its free form and/or when it is bound to a PSP94-binding protein.

In order to promote the formation of a PSP94/PSP94-binding protein complex, the two (substantially or partially) purified proteins were pre-incubated together. Briefly, a partially pure PSP94-binding protein preparation (see example 4), at a concentration of 1 mg/ml (total protein concentration) in PBS containing 0.5% BSA was pre-incubated for 1 hour at 34° C. with or without 5 μg/ml of native PSP94.

An ELISA plate (96 well plate) was coated with 17G9 monoclonal antibody at a concentration of 2 μg/ml (in 0.1 M NaHCO₃ pH 8.0) by an overnight incubation at 4° C. As described herein, this antibody recognizes a PSP94-binding protein. Wells of the plate were subsequently blocked with 1% BSA for 1 hour at 34° C. The PSP94/PSP94-binding protein complex generated above was incubated with the 17G9 coated plates for 1 hour at 34° C. before washing off any unbound material. The plates were then incubated with biotinylated PSP94-specific antibodies (2 μg/ml in PBS 0.5% BSA). Any positive binding of these antibodies would indicate that the PSP94 epitope that is recognized is exposed (available) even when bound to a PSP94-binding protein. These results are illustrated in table 10. Binding of the biotinylated PSP94-specific antibodies to the bound PSP94 was visualized with a streptavidin peroxidase system and developed with TMB giving a blue color.

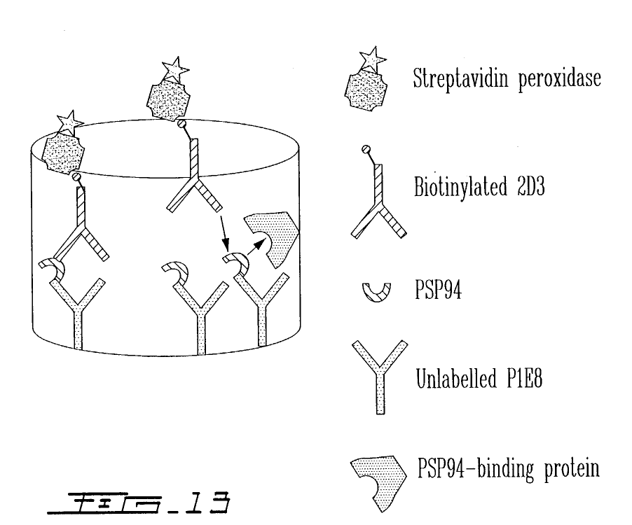

Results illustrated in FIG. 11 indicate that none of the antibodies tested react with captured PSP94-binding protein when the binding sites are not saturated with PSP94. When the binding sites are saturated with PSP94, P1E8 shows strong reactivity towards the complex. However, 2D3 and 12C3 do not. Thus, P1E8 recognize bound and free PSP94 and the other two antibodies (2D3 and 12C3) only recognize the free form of the protein. Antibodies 2D3 and 12C3 probably recognize a PSP94 epitope that is masked when it is bound to a PSP94-binding protein. Each of these antibodies detects native and recombinant PSP94 when coated onto ELISA plates. All three antibodies function as capture or detector antibodies in sandwich ELISA formats to produce a linear standard curve over a useful range of concentrations of PSP94. However, 12C3 appears to be of lower affinity than 2D3 or P1E8 toward PSP94.

The utility of these antibodies to detect PSP94 was illustrated in the following assay; an ELISA plate was coated with 5 μg/ml of PSP94 in pH 9.6 carbonate buffer and incubated overnight at 4° C. The plate was blocked with 1% BSA for 1 h at 34° C. Samples were then incubated in the plate overnight at 4° C. Biotinylated P1E8 was applied at 1 microgram/ml for 2 hrs at 34° C. and peroxidase streptavidin was applied for 1 h at 34° C. before development in TMB. The lower limit of quantification (LLQ) was shown to be in the range of 1 ng/ml. It is of particular interest that the assay (e.g., standard curve) may be performed with native PSP94 (i.e., PSP94 isolated from human serum) or recombinant PSP94.

### EXAMPLE 13

### Free PSP94 Immunodetection Assays

The three PSP94 monoclonal antibodies described above (2D3 (PTA-4240), P1E8 (PTA-4241), 12C3), may be used in competitive ELISA assays (i.e., coating plates with PSP94 (or sample), and using the PSP94 within the sample to inhibit the binding of the monoclonal antibody to the PSP94 coated plates). The use of 2D3 in a competitive ELISA format was investigated.

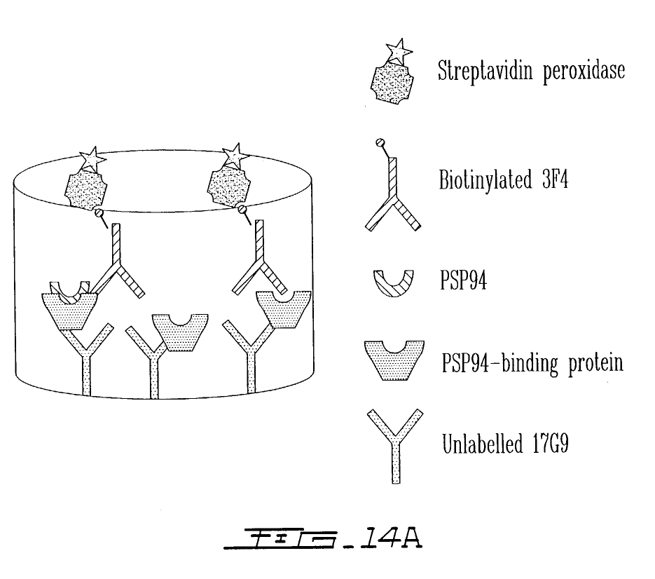

An example of an ELISA assay to measure free PSP94, involves coating the ELISA plates with the 2D3 antibody. The coated plates may then be incubated with samples, and PSP94 may be detected with biotinylated P1E8, since 2D3 and P1E8 recognize different PSP94 epitopes. FIG. 12b represent results of an ELISA assay using the method illustrated in FIG. 12a.

In order to limit the possible dissociation (e.g., promoted by 2D3) of the PSP94/PSP94-binding protein complex during the ELISA assay, improvements were introduced. Briefly, the improved assay involves pre-absorption (removal) of the PSP94/PSP94-binding protein complex with a PSP94-binding protein antibody before performing the assay. The PSP94-binding protein antibodies selectively remove PSP94-binding protein and the PSP94/PSP94-binding protein complex (i.e., bound PSP94). This is done without upsetting the kinetics of the equilibrium reaction between a PSP94-binding protein and PSP94. Pre-absorption can be done with, for example the 17G9 linked to a sepharose matrix, giving then a sample that is free of the complex (unbound PSP94 remains). The sample is then processed as described above (i.e., incubating the complex-free sample with the plate coated with 2D3 and detecting with biotinylated P1E8.

### EXAMPLE 14

### Total PSP94 Immunodetection Assays

Since the P1E8 antibody is able to recognize PSP94 both in its free and bound form, an assay to measure total PSP94 has been developed. For example, P1E8 is immobilized to the plate and a sample containing free PSP94 and PSP94 complexed with a PSP94-binding protein is added. The PSP94 and the complex remains bound to the antibody and an antibody having a different affinity (a different binding site on PSP94) than P1E8 may be added. An example of such an antibody is 2D3 or any other suitable PSP94-antibody. Detection is performed by using a label that may be conjugated to 2D3 or by a secondary molecules (antibody or protein) recognizing directly or indirectly (e.g., biotin/avidin or streptavidin system) the 2D3 antibody.

However, based on the observation that 2D3 might disturb the binding equilibrium between PSP94 and PSP94-binding protein, the assay to measure total PSP94 (bound and unbound) was improved.

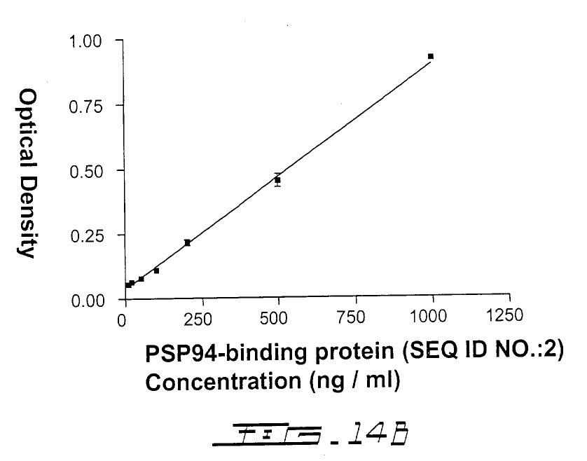

Particularly, the assay was performed as illustrated in FIG. 13. In FIG. 13, total PSP94 is captured with the P1E8 antibody, and a high concentration (excess) of biotinylated 2D3 is used to encourage the dissociation (displacement) of a PSP94-binding protein. In the previously described assay, the actual concentration of 2D3 for coating the plate is low as the plastic has a capacity of no more than 50 ng.

Note, that this assay may also measure free (unbound) PSP94, if the complex (PSP94/PSP94-binding protein) is adsorbed out from the serum prior to measurement.

### Example 15

### PSP94-Binding Protein Immunodetection Assays

Specificity for all the PSP94-binding protein antibodies has been confirmed in the ELISA assay discussed previously, and by Western blot. Each of them recognizes both the high and low molecular weight form of the binding protein by western blot.

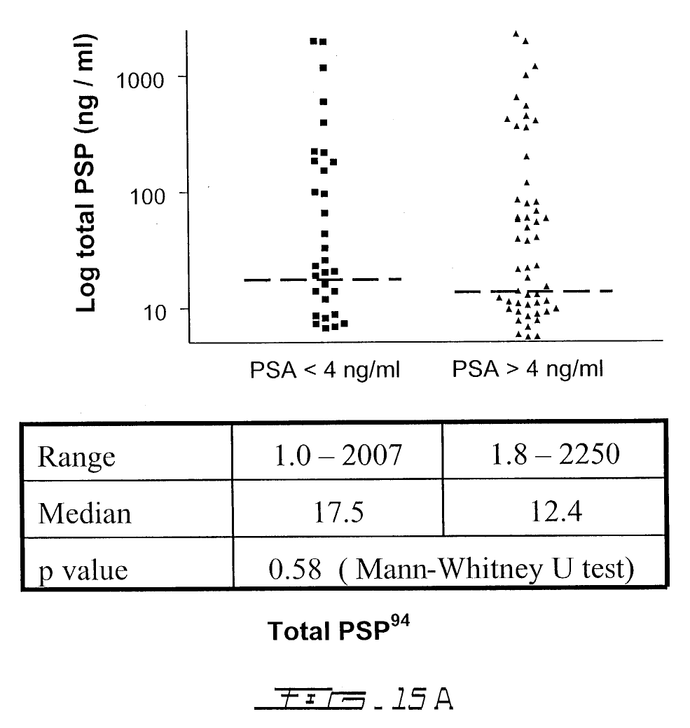

As shown in table 10, the antibody 17G9 recognize a different epitope than 3F4. Thus a sandwich ELISA assay, as illustrated in FIG. 14a, has been developed using these two antibodies. FIG. 14b illustrates a standard curve from the assays used to measure a PSP94-binding protein within serum samples. Note that these two antibodies may be interchanged. For example, the capture antibody can be switched to be used as detection reagent (when labeled).

Forty serum samples from male donors have been assessed with a PSP94-binding protein ELISA assay described above (illustrated in FIG. 14a). The PSP94-binding protein serum concentration was successfully measured. Values of PSP94-binding protein in these male donors ranged from about 1 μg/ml to about 10 μg/ml, with two cases having in excess of 20 μg/ml. Two cases from female donors have been assessed; one has about 3 μg/ml, the other about 7.8 μg/ml.

### EXAMPLE 16

### Immunodetection Assays Application

Male human serum samples with known total PSA values were obtained from a reference standard laboratory. Forty cases had low total PSA serum levels (<4 ng per ml) and 69 had high total PSA serum levels (>4 ng per ml). Analysis was performed on these low and high categories. There is no traceable link back to these patients, thus, there is no clinical information associated with the specimens, except for the total PSA value. The purpose of this analysis is to look for trends and patterns rather than determine the clinical relevance of PSP94 measurements. The distributions of the serum concentrations of total PSP94, PSP94-binding protein, free PSP94 and corrected free PSP94 are illustrated in additional figures described herein.

With respect to additional figures;

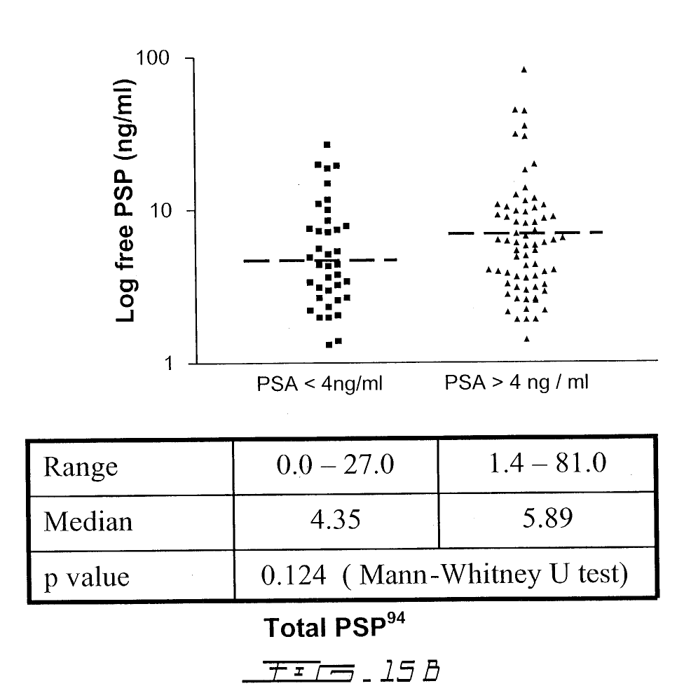

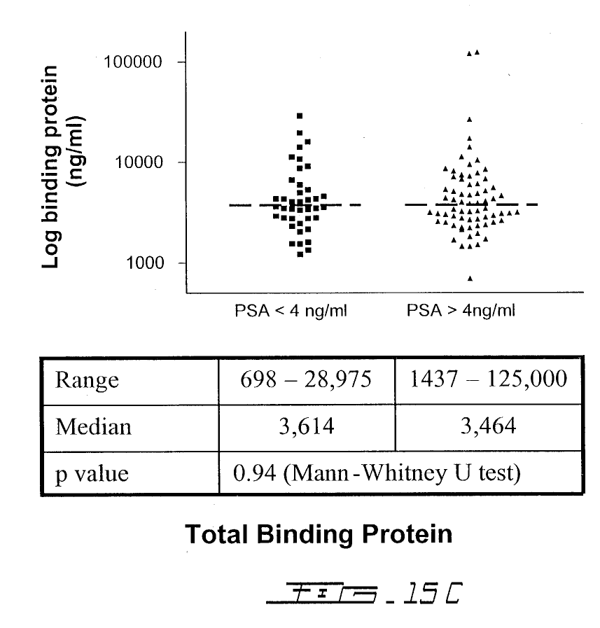

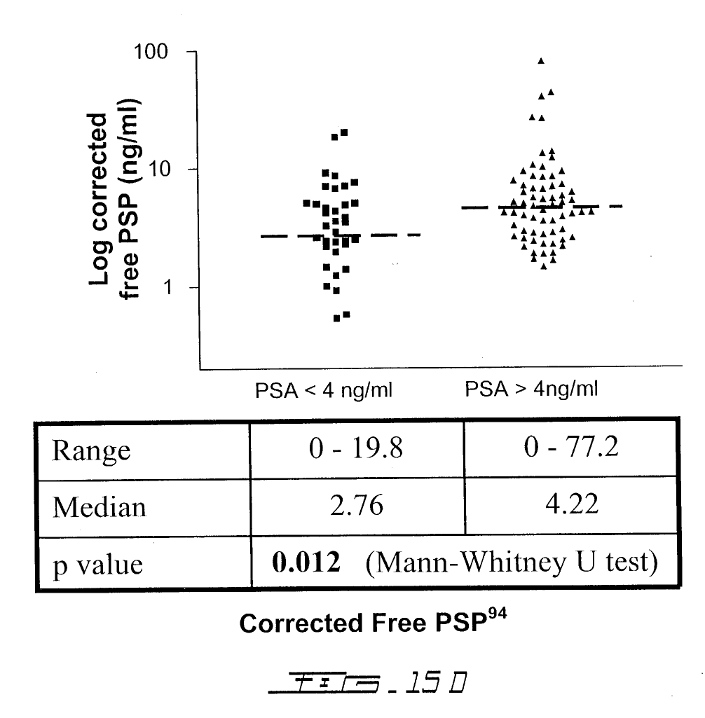

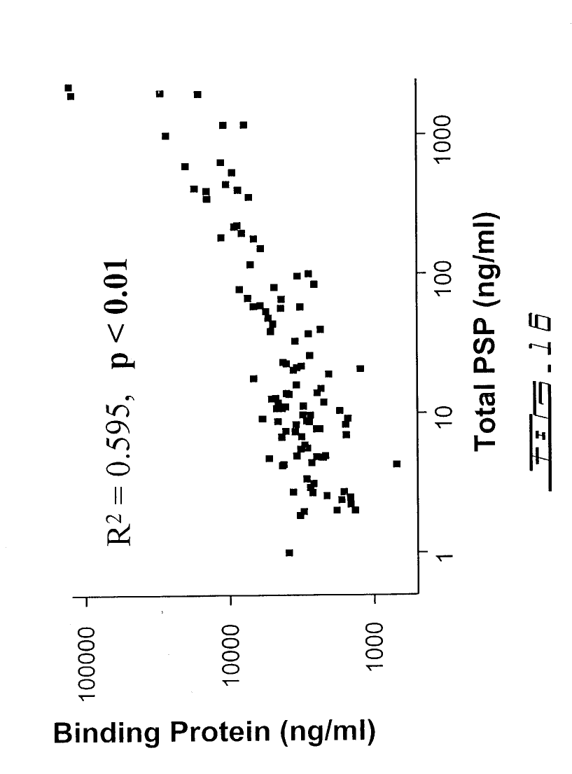

FIG. 15A, is a graph illustrating results obtained following measurement of total PSP94 in serum of individuals for which PSA values are known to be lower or higher than the cut-off value of 4 ng/ml and using an assay as illustrated in FIG. 13 and described in example 14. Results are expressed as the log of total PSP94 concentration (in ng/ml) measured for each individual. Each point represent results obtained for a specific individual. With respect to this figure, total PSP94 concentration of 1 to 2250 ng/ml were measured in serum of individuals.

With respect to FIG. 15B, this figure is a graph illustrating results obtained following measurement of free PSP94 in serum of individuals for which PSA values are known to be lower or higher than the cut-off value of 4 ng/ml. Results were obtained using an assay which is based on the removal (depletion) of PSP94-binding protein and PSP94/PSP94-binding protein complex from serum using an anti-PSP94-binding protein antibody as described herein prior to measurement of free PSP94 with the 2D3 and P1E8 monoclonal antibodies in a sandwich ELISA assay. Results are expressed as the log of free PSP94 concentration (in ng/ml) measured for each individual. Each point represent results obtained for a specific individual.

With respect to FIG. 15C, this figure is a graph illustrating results obtained following measurement of total PSP94-binding protein in serum of individuals for which PSA values are known to be lower or higher than the cut-off value of 4 ng/ml. Results were obtained using an assay which is illustrated in FIG. 14a and described in example 15. Results are expressed as the log of total PSP94-binding protein concentration (in ng/ml) measured for each individual. Each point represent results obtained for a specific individual. With respect to this figure, PSP94-binding protein concentration ranging from 0.7 to 125 micrograms/ml were measured in serum of individuals.

With respect to FIG. 15D, this figure is a graph illustrating results obtained following correction of the free PSP94 concentration obtained in serum of individuals for which PSA values are known to be lower or higher than the cut-off value of 4 ng/ml. Results were corrected by taking into account that 1 to 5% of residual PSP94/PSP94-binding protein complex remains in the serum even after depletion which may affect the results obtain, i.e., PSP94 may be dissociated from the complex after the 2D3 antibody is added, falsely increasing the “free PSP94” value. Results are again expressed as the log of corrected free PSP94 concentration (in ng/ml) measured for each individual. Each point represent results obtained for a specific individual. With respect to this figure, corrected free PSP94 levels were significantly elevated in the high PSA category (>4 ng/ml).

FIG. 16, is a graph illustrating the total PSP94-binding protein concentration (ng/ml) versus the total PSP94 concentration (ng/ml) measured in serum of individuals, where each point represent results obtained for a specific individual. With respect to this figure, a significant positive relationship between these two parameters may be observed.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

We claim:

1. A polynucleotide comprising a member selected from the group consisting of
a) a polynucleotide as set forth in SEQ ID NO.: 1,
b) a polynucleotide as set forth in SEQ ID NO.: 6,
c) a polynucleotide having sequence 1 to 1392 of SEQ ID NO.:6,
d) a polynucleotide having sequence 1 to 1653 of SEQ ID NO.:6,
e) a polynucleotide of a size between 10 and 2005 bases in length identical in sequence to a contiguous portion of at least 10 bases of the polynucleotide as set forth in SEQ ID NO.: 1, and
f) a polynucleotide of a size between 10 and 1876 bases in length identical in sequence to a contiguous portion of at least 10 bases of the polynucleotide as set forth in SEQ ID NO.: 6.

2. The polynucleotide as defined in claim 1, wherein said polynucleotide is as set forth in SEQ ID NO.: 1.

3. The polynucleotide as defined in claim 1, wherein said polynucleotide is as set forth in SEQ ID NO.: 6.

4. The polynucleotide as defined in claim 1, wherein said polynucleotide is the has sequence 1 to 1392 of SEQ ID NO.:6.

5. The polynucleotide as defined in claim 1, wherein said polynucleotide is the has sequence 1 to 1653 of SEQ ID NO.:6.

6. The polynucleotide as defined in claim 1, wherein said polynucleotide is selected from the group consisting of a polyribonucleotide, a polydeoxyribonucleotide, a modified polyribonucleotide, a modified polydeoxyribonucleotide and a complementary polynucleotide.

7. An isolated polypeptide selected from the group consisting of
a) a polypeptide as set forth in SEQ ID NO.: 2,
b) a polypeptide as set forth in SEQ ID NO.: 3,
c) a polypeptide as set forth in SEQ ID NO.: 7,
d) a polypeptide as set forth in SEQ ID NO.: 8,
e) a polypeptide as set forth in SEQ ID NO.: 9,
f) a polypeptide of a size between 10 and 505 amino acids in length identical to a contiguous portion of the same size of SEQ ID NO.:2,
g) a polypeptide of a size between 10 and 592 amino acids in length identical to a contiguous portion of the same size of SEQ ID NO.:3,
h) a polypeptide of a size between 10 and 624 amino acids in length identical to a contiguous portion of the same size of SEQ ID NO.:7,
i) a polypeptide analogue having at least 90% of its amino acid sequence identical to the amino acid sequence set forth in SEQ ID NO.:2, in SEQ ID NO.:3, in SEQ ID NO.:7 in SEQ ID NO.:8 or in SEQ ID NO.:9,
j) a polypeptide analog having at least 70% of its amino acid sequence identical to the amino acid sequence set forth in SEQ ID NO.: 2, in SEQ ID NO.:3, in SEQ ID NO.:7, in SEQ ID NO: 8 or in SEQ ID NO.:9,
k) a polypeptide analog having at least 50% of its amino acid sequence identical to the amino acid sequence set forth in SEQ ID NO: 2 in SEQ ID NO.:3, in SEQ ID NO.:7, in SEQ ID NO: 8 or in SEQ ID NO.:9,
l) a polypeptide analogue having at least 90% of its amino acid sequence identical to the amino acid sequence of
a polypeptide of a length from between 10 and 505 contiguous amino acids of SEQ ID NO.:2,
a polypeptide of a length from between 10 and 592 contiguous amino acids of SEQ ID NO.:3 or,
a polypeptide of a length from between 10 and 624 contiguous amino acids of SEQ ID NO.:7,
m) a polypeptide analogue having at least 70% of its amino acid sequence identical to the amino acid sequence of
a polypeptide of a length from between 10 and 505 contiguous amino acids of SEQ ID NO.:2,
a polypeptide of a length from between 10 and 592 contiguous amino acids of SEQ ID NO.:3 or,
a polypeptide of a length from between 10 and 624 contiguous amino acids of SEQ ID NO.:7,
n) a polypeptide analogue having at least 50% of its amino acid sequence identical to the amino acid sequence of
a polypeptide of a length from between 10 and 505 contiguous amino acids of SEQ ID NO.:2,
a polypeptide of a length from between 10 and 592 contiguous amino acids of SEQ ID NO.:3 or,
a polypeptide of a length from between 10 and 624 contiguous amino acids of SEQ ID NO.:7.

8. A polypeptide as defined in claim 7, wherein said polypeptide is as set forth SEQ ID NO.: 2.

9. A polypeptide as defined in claim 7, wherein said polypeptide is as set forth SEQ ID NO.: 3.

10. A polypeptide as defined in claim 7, wherein said polypeptide is as set forth SEQ ID NO.:7.

11. A polypeptide as defined in claim 7, wherein said polypeptide is as set forth SEQ ID NO.:8.

12. A polypeptide as defined in claim 7, wherein said polypeptide is as set forth SEQ ID NO.:9.

13. An immunizing composition including;
a) a vector comprising a polynucleotide as defined in claim 1 and;
b) a diluent or buffer.

14. An immunizing composition as defined in claim 13, further comprising an adjuvant.

15. An immunizing composition as defined in claim 14, further comprising PSP94, a PSP94 variant, a PSP94 fragment, a polynucleotide encoding PSP94, a polynucleotide encoding a PSP94 variant, a polynucleotide encoding a PSP94 fragment and combination thereof.

16. An immunizing composition comprising;
a) a polypeptide as defined in claim 7, and;
b) a diluent or buffer.

17. An immunizing composition as defined in claim 16, further comprising an adjuvant.

18. An immunizing composition as defined in claim 16, further comprising PSP94, a PSP94 variant, a PSP94 fragment, a polynucleotide encoding PSP94, a polynucleotide encoding a PSP94 variant, a polynucleotide encoding a PSP94 fragment and combination thereof.

19. A method of generating an antibody to a polypeptide, said method comprising administering to a mammal, an immunizing composition as defined in claim 15.

20. A method of generating an antibody to a polypeptide, said method comprising administering to a mammal, an immunizing composition as defined in claim 18.

21. A cell that has incorporated at least one of the polynucleotide defined in claim 1.

22. A cell that has incorporated at least one of the polypeptide defined in claim 7.

23. A cell expressing at least one of the polypeptide defined in claim 7.

24. The use of a polynucleotide as defined in claim 1, in the diagnosis or prognosis of a condition linked with elevated levels of PSP94 or PSP94-binding protein.

25. The use as defined in claim 24, wherein said polynucleotide is as set forth in SEQ ID NO.:1.

26. The use as defined in claim 24, wherein said polynucleotide is as set forth in SEQ ID NO.:6.

27. The use as defined in claim 24, wherein said polynucleotide has sequence 1 to 1392 of SEQ ID NO.:6.

28. The use as defined in claim 24, wherein said polynucleotide has sequence 1 to 1653 of SEQ ID NO.:6.

29. The use of a polypeptide as defined in claim 7 in the diagnosis or prognosis of a condition linked with elevated levels of PSP94 or PSP94-binding protein.

30. The use as defined in claim 29, wherein said polypeptide is selected from the group consisting of SEQ ID NO.: 2, SEQ ID NO.: 3, SEQ ID NO.:7, SEQ ID NO.:8 and SEQ ID NO.:9.

31. The monoclonal antibody produced by the hybridoma cell line deposited to the ATCC under Patent Deposit No.: PTA-4242 and antigen binding fragments thereof.

32. The monoclonal antibody produced by the hybridoma cell line deposited to the ATCC under Patent Deposit No.: PTA-4243 and antigen binding fragments thereof.

33. The hybridoma cell line deposited to the ATCC under Patent Deposit No.: PTA-4242.

34. The hybridoma cell line deposited to the ATCC under Patent Deposit No.: PTA-4243.

35. A method for measuring, in a sample, the amount of a polypeptide selected from the group consisting of SEQ ID NO.: 2, SEQ ID NO.: 3, SEQ ID NO.:7, SEQ ID NO.:8 and SEQ ID NO.:9 or combination thereof, said method comprising contacting said sample with a molecule able to recognize said polypeptide.

36. The method of claim 35, wherein said molecule is an antibody selected from the group consisting of the monoclonal antibody produced by the hybridoma cell line deposited to the ATCC under Patent Deposit No.: PTA-4242 and the monoclonal antibody produced by the hybridoma cell line deposited to the ATCC under Patent Deposit No.: PTA-4243.

37. The method of claim 35, wherein said molecule is PSP94 and analogues thereof.

38. The method of claim 35, further comprising detecting a signal from a label that is provided by said molecule or by a second molecule carrying said label.

39. The method of claim 38, wherein the signal obtained for the sample is compared with a signal obtained for a control sample containing a known amount of at least one polypeptide selected from the group consisting of SEQ ID NO.: 2, SEQ ID No.:3, SEQ ID NO.:7, SEQ ID NO.:8 and SEQ ID NO.:9 or combination thereof.

40. A method for measuring, in a sample, the amount of a polypeptide selected from the group consisting of SEQ ID NO.: 2, SEQ ID NO.:3, SEQ ID NO.:7, SEQ ID NO.:8 and SEQ ID NO.:9 or combination thereof, that is not bound to PSP94, said method comprising;
a) removing, from said sample, a complex formed by PSP94 and any one of the polypeptide selected from the group consisting of SEQ ID NO.: 2, SEQ ID NO.:3, SEQ ID NO.:7, SEQ ID NO.:8 and SEQ ID NO.:9, generating a complex-free sample, and;
b) contacting said complex-free sample with an antibody able to recognize any one of the polypeptide selected from the group consisting of SEQ ID NO.: 2, SEQ ID NO.:3, SEQ ID NO.:7, SEQ ID NO.:8 and SEQ ID NO.:9.

41. The method of claim 40, wherein said antibody is selected from the group consisting of the monoclonal antibody produced by the hybridoma cell line deposited to the ATCC under Patent Deposit No.: PTA-4242 and the monoclonal antibody produced by the hybridoma cell line deposited to the ATCC under Patent Deposit No.: PTA-4243.

42. The method of claim 40, further comprising detecting a signal from a label that is provided by said antibody or by a second molecule carrying said label.

43. The method of claim 42, wherein the signal obtained for the sample is compared with signal obtained for a control sample containing a known amount of a polypeptide selected from the group consisting of SEQ ID NO.: 2, SEQ ID NO.:3, SEQ ID NO.:7, SEQ ID NO.:8 and SEQ ID NO.:9.

44. The use of an antibody selected from the group consisting of a monoclonal antibody produced by the hybridoma cell line deposited to the ATCC under Patent Deposit No.: PTA-4240, a monoclonal antibody produced by the hybridoma cell line deposited to the ATCC under Patent Deposit No.: PTA-4241, a monoclonal antibody produced by the hybridoma cell line deposited to the ATCC under Patent Deposit No.: PTA-4242 and a monoclonal antibody produced by the hybridoma cell line deposited to the ATCC under Patent Deposit No.: PTA-4243, for evaluating the amount of SEQ ID NO.:2, SEQ ID NO.: 3 SEQ ID NO.:7, SEQ ID NO.:8 and SEQ ID NO.:9 or combination thereof.

45. The use of a molecule selected from the group consisting of a polypeptide as set forth in SEQ ID NO.:2, SEQ ID NO.: 3, SEQ ID NO.:7, SEQ ID NO.:8 and SEQ ID NO.:9, a monoclonal antibody produced by the hybridoma cell line deposited to the ATCC under Patent Deposit No.: PTA-4240, a monoclonal antibody produced by the hybridoma cell line deposited to the ATCC under Patent Deposit No.: PTA-4241, a monoclonal antibody produced by the hybridoma cell line deposited to the ATCC under Patent Deposit No.: PTA-4242 and a monoclonal antibody produced by the hybridoma cell line deposited to the. ATCC under Patent Deposit No.: PTA-4243, for evaluating the amount of PSP94 or for the diagnostic of a condition linked with abnormal or elevated levels of PSP94.

46. The use as defined in claim 45, wherein said condition is selected from the group consisting of prostate cancer, stomach cancer, breast cancer, endometrial cancer, ovarian cancer, other cancers of epithelial secretion and benign prostate hyperplasia.

47. An antibody conjugate comprising a first moiety and a second moiety, said first moiety being selected from the group consisting of a monoclonal antibody produced by the hybridoma cell line deposited to the ATCC under Patent Deposit No.: PTA-4240, a monoclonal antibody produced by the hybridoma cell line deposited to the ATCC under Patent Deposit No.: PTA-4241, a monoclonal antibody produced by the hybridoma cell line deposited to the ATCC under Patent Deposit No.: PTA-4242 and a monoclonal antibody produced by the hybridoma cell line deposited to the ATCC under Patent Deposit No.: PTA-4243 and said second moiety being selected from the group consisting of a pharmaceutical agent, a solid support, a reporter molecule, a group carrying a reporter molecule, a chelating agent, an acylating agent, a cross-linking agent, and a targeting group.

48. The conjugate of claim 47, wherein said solid support is selected from the group consisting of carbohydrates, liposomes, lipids, colloidal gold, microparticles, microcapsules, microemulsions, and the matrix of an affinity column.

49. The conjugate of claim 47, wherein said reporter molecule is selected from the group consisting of a fluorophore, a chromophore, a dye, an enzyme, a radioactive molecule and a molecule of a binding/ligand complex.

50. The conjugate of claim 47, wherein said pharmaceutical agent is selected from the group of a toxin, a drug and a pro-drug.

51. A kit for use in evaluating the amount of PSP94 or for the diagnosis of a condition linked with abnormal or elevated levels of PSP94 comprising a container having a molecule able to recognize PSP94.

52. The kit of claim 51, wherein said molecule is selected from the group consisting of a monoclonal antibody produced by the hybridoma cell line deposited to the ATCC under Patent Deposit No.: PTA-4240, a monoclonal antibody produced by the hybridoma cell line deposited to the ATCC under Patent Deposit No.: PTA-4241, a monoclonal antibody produced by the hybridoma cell line deposited to the ATCC under Patent Deposit No.: PTA-4242, a monoclonal antibody produced by the hybridoma cell line deposited to the ATCC under Patent Deposit No.: PTA-4243 and the antibody conjugate of claim 47.

53. The kit of claim 51, wherein said molecule is selected from the group consisting of the polypeptide set forth in SEQ ID NO.:2, the polypeptide set forth in SEQ ID NO.:3, the polypeptide set forth in SEQ ID NO.:7, the polypeptide set forth in SEQ ID NO.:8 and the polypeptide set forth in SEQ ID NO.:9.

54. The kit of claim 53, further comprising a container having an antibody able to recognize a polypeptide selected from the group consisting of the polypeptide set forth in SEQ ID NO.:2, the polypeptide set forth in SEQ ID NO.:3, the polypeptide set forth in SEQ ID NO.:7, the polypeptide set forth in SEQ ID NO.:8 and the polypeptide set forth in SEQ ID NO.:9.

55. The kit of claim 54, wherein said antibody is selected from the group consisting of a monoclonal antibody produced by the hybridoma cell line deposited to the ATCC under Patent Deposit No.: PTA-4243 and a monoclonal antibody produced by the hybridoma cell line deposited to the ATCC under Patent Deposit No.: PTA-4242

56. A method for preparing a polypeptide selected from the group consisting of the polypeptide set forth in SEQ ID NO.:2, the polypeptide set forth in SEQ ID NO.:3, the polypeptide set forth in SEQ ID NO.:7, the polypeptide set forth in SEQ ID NO.:8 and the polypeptide set forth in SEQ ID NO.:9 comprising:
a) cultivating a host cell under conditions which provide for the expression of said polypeptide by the cell; and
b) recovering the polypeptide by one or more purification step.

57. The method of claim 56, wherein said purification step either alone or in combination is selected from the group consisting of ammonium sulfate precipitation, size exclusion chromatography, affinity chromatography, and ion-exchange chromatography.

58. A method for preparing a polypeptide selected from the group consisting of the polypeptide set forth in SEQ ID NO.:2, the polypeptide set forth in SEQ ID NO.:3, the polypeptide set forth in SEQ ID NO.:7, the polypeptide set forth in SEQ ID NO.: 8, the polypeptide set forth in SEQ ID NO.:9 and combination thereof, comprising:
a) collecting one or more biological sample containing said polypeptide; and
b) recovering the polypeptide by one or more purification step.

59. The method of claim 58, wherein said purification step either alone or in combination is selected from the group consisting of ammonium sulfate precipitation, size exclusion chromatography, affinity chromatography, and ion-exchange chromatography.

60. The method of claim 58, wherein said purification step comprises;
a) adding ammonium sulfate to said biological sample,
b) performing ion-exchange chromatography,
c) performing affinity-chromatography using a PSP94-conjugated affinity matrix,
d) performing size-exclusion chromatography, and
e) recovering a fraction containing a substantially pure PSP94-binding protein.

61. The method of claim 58, wherein said biological sample is a serum sample, a plasma sample, a blood sample and a cell lyzate sample.

62. A process for the purification of a PSP94-binding protein from a sample comprising:
a) adding ammonium sulfate to said sample in a manner as to provide precipitation of a PSP94-binding protein,
b) centrifuging the mixture of step a) to recover precipitated proteins,
c) resuspending said precipitated proteins,
d) performing ion-exchange chromatography to recover a fraction of proteins containing a PSP94-binding protein,
e) performing affinity-chromatography using a PSP94-conjugated affinity matrix to recover a fraction of proteins containing a PSP94-binding protein,
f) performing size exclusion chromatography to recover a fraction of proteins containing a PSP94-binding protein and;
g) recovering a fraction containing a substantially pure PSP94-binding protein.

63. The process of claim 62, wherein said sample is human male serum.

64. The process of claim 62, wherein the precipitation of a PSP94-binding protein is effected by adding ammonium sulfate to a final concentration of up to 47%.

65. The process of claim 62, wherein said ion-exchange chromatography is performed by using an anion-exchange chromatography matrix.

66. The process of claim 62, wherein said PSP94-binding protein is a polypeptide selected from the group consisting of the polypeptide defined in SEQ ID NO.:2, the polypeptide defined in SEQ ID NO.:3, the polypeptide defined in SEQ ID NO.:7, the polypeptide defined in SEQ ID NO.:8 and the polypeptide defined in SEQ ID NO.:9.

67. The product obtained from the process of claim 62.

68. An antibody able to recognize a PSP94 epitope that is available even when PSP94 is bound to another polypeptide.

69. The antibody as defined in claim 68, wherein said polypeptide is selected from the group consisting of SEQ ID NO.: 2, SEQ ID NO.: 3, SEQ ID NO.: 7, SEQ ID NO.:8 and SEQ ID NO.:9.

70. An antibody as defined in claim 68, wherein said antibody is the monoclonal antibody produced by the hybridoma cell line deposited to the ATCC under Patent Deposit NO.: PTA-4241.

71. A hybridoma cell line producing the antibody defined in claim 68.

72. The monoclonal antibody produced by the hybridoma cell line deposited to the ATCC under Patent Deposit No.: PTA-4240 and antigen binding fragments thereof.

73. The monoclonal antibody produced by the hybridoma cell line deposited to the ATCC under Patent Deposit No.: PTA-4241 and antigen binding fragments thereof.

74. The hybridoma cell line deposited to the ATCC under Patent Deposit No.: PTA-4240.

75. The hybridoma cell line deposited to the ATCC under Patent Deposit No.: PTA-4241.

76. A method for removing PSP94 from a sample, said method comprising;
a) contacting said sample with a molecule able to bind to PSP94, and;
b) recuperating a sample free of PSP94.

77. The method of claim 76, wherein said molecule is selected from the group consisting of SEQ ID NO.: 2, SEQ ID NO.:3, SEQ ID NO.: 7, SEQ ID NO.:8, SEQ ID NO.:9, a monoclonal antibody produced by the hybridoma cell line deposited to the ATCC under Patent Deposit No.: PTA-4240 and a monoclonal antibody produced by the hybridoma cell line deposited to the ATCC under Patent Deposit No.: PTA-4241.

78. The method of claim 76, wherein said sample is selected from the group consisting of blood, plasma, serum, urine, seminal fluid, cell culture media and cell lyzate.

79. A method for removing a complex formed by PSP94 and any one of the polypeptide defined in SEQ ID NO: 2, SEQ ID NO.:3, SEQ ID NO.:7, SEQ ID NO.:8 or SEQ ID NO.: 9 and combination thereof from a sample, said method comprising;
a) contacting said sample with an antibody able to recognize an exposed epitope of said complex, and;
b) recuperating a sample free of said complex.

80. The method of claim 79, wherein said antibody is selected from the group consisting of a monoclonal antibody produced by the hybridoma cell line deposited to the ATCC under Patent Deposit No.: PTA-4241, a monoclonal antibody produced by the hybridoma cell line deposited to the ATCC under Patent Deposit No.: PTA-4242 and a monoclonal antibody produced by the hybridoma cell line deposited to the ATCC under Patent Deposit No.: PTA-4243.

81. The method of claim 79, wherein said antibody is the monoclonal antibody produced by the hybridoma cell line deposited to the ATCC under Patent Deposit No.: PTA-4243.

82. A method for measuring, in a sample, the total amount of PSP94, said method comprising contacting said sample with an antibody able to recognize PSP94 even when PSP94 is bound to another polypeptide.

83. The method of claim 82, wherein said antibody is the monoclonal antibody produced by the hybridoma cell line deposited to the ATCC under Patent Deposit No.: PTA-4241.

84. The method of claim 82, further comprising detecting a signal from a label that is provided by said antibody or by a second molecule carrying said label.

85. The method of claim 84, wherein the signal obtained for the sample is compared with signal obtained for a control sample containing a known amount of PSP94, PSP94 fragments, variants or analogues thereof.

86. An improved method for measuring the amount of free PSP94 in a sample, said method comprising;
a) removing a complex formed by PSP94 and any one of the polypeptide selected from the group consisting of SEQ ID NO.: 2, SEQ ID NO.:3, SEQ ID NO.:7, SEQ ID NO.:8 and SEQ ID NO.:9 and combination thereof, generating a complex-free sample
b) contacting said complex-free sample with an antibody able to recognize PSP94.

87. The method of claim 86, wherein said antibody is selected from the group consisting of the monoclonal antibody produced by the hybridoma cell line deposited to the ATCC under Patent Deposit No.: PTA-4240 and the monoclonal antibody produced by the hybridoma cell line deposited to the ATCC under Patent Deposit No.: PTA-4241.

88. The method of claim 86, further comprising detecting a signal from a label that is provided by said antibody or by a second molecule carrying said label.

89. The method of claim 88, wherein the signal obtained for the sample is compared with signal obtained for a control sample containing a known amount of PSP94.

90. An improved method for measuring the amount of free PSP94 in a sample, said method comprising contacting said sample with an antibody able to recognize PSP94.

91. The method of claim 90, wherein said antibody is selected from the group consisting of the monoclonal antibody produced by the hybridoma cell line deposited to the ATCC under Patent Deposit No.: PTA-4240 and the monoclonal antibody produced by the hybridoma cell line deposited to the ATCC under Patent Deposit No.: PTA-4241.

92. The method of claim 90, further comprising detecting a signal from a label that is provided by said antibody or by a second molecule carrying said label.

93. The method of claim 92, wherein the signal obtained for the sample is compared with signal obtained for a control sample containing a known amount of PSP94.

94. A method for measuring the level of total PSP94 in a sample, the method comprising using a first and a second antibody able to bind to PSP94 even when PSP94 is bound to a polypeptide and wherein said first and second antibody binds to a different PSP94 epitope.

95. A method for measuring the levels of PSP94 in a sample said method comprising contacting said sample with an antibody that is able to recognize PSP94 in its free and bound form.

96. The method of claim 95, wherein said antibody is the monoclonal antibody produced by the hybridoma cell line deposited to the ATCC under Patent Deposit NO.: PTA-4241.

97. A method for measuring total PSP94 in a sample, the method comprising using a first and a second antibody, wherein said first antibody is able to bind to PSP94 even when PSP94 is bound to another polypeptide and wherein said second antibody is able to bind to PSP94 and to displace any one of the polypeptide selected from the group consisting of SEQ ID NO.:2, SEQ ID NO.:3, SEQ ID NO.:7, SEQ ID NO.:8 and SEQ ID NO.:9 from a complex formed by PSP94 and said polypeptide.

98. The method of claim 97, wherein said first antibody is the monoclonal antibody produced by the hybridoma cell line deposited to the ATCC under Patent Deposit No.: PTA-4241.

99. The method of claim 97, wherein said second antibody is the monoclonal antibody produced by the hybridoma cell line deposited to the ATCC under Patent Deposit No.: PTA-4240.

100. The use of a molecule selected from the group consisting of the polypeptide as set forth in SEQ ID NO.:2, SEQ ID NO.:3, SEQ ID NO.:7, SEQ ID NO.:8 or SEQ ID NO.: 9, a monoclonal antibody produced by the hybridoma cell line deposited to the ATCC under Patent Deposit No.: PTA-4240, a monoclonal antibody produced by the hybridoma cell line deposited to the ATCC under Patent Deposit No.: PTA-4241, a monoclonal antibody produced by the hybridoma cell line deposited to the ATCC under Patent Deposit No.: PTA-4242 and a monoclonal antibody produced by the hybridoma cell line deposited to the ATCC under Patent Deposit No.: PTA-4243, for evaluating the amount of PSP94, PSP94 variants and analogues thereof in a sample.

101. The use of a PSP94 antibody for the treatment of a condition associated with elevated levels of PSP94.

102. The use as defined in claim 101, wherein said antibody is selected from the group consisting of a monoclonal antibody produced by the hybridoma cell line deposited to the ATCC under Patent Deposit No.: PTA-4240 and a monoclonal antibody produced by the hybridoma cell line deposited to the ATCC under Patent Deposit No.: PTA-4241.

103. The use of a PSP94 antibody in the manufacture of a medicament for the treatment of a condition associated with elevated levels o PSP94.

104. The use as defined in claim 103, wherein said antibody is selected from the group consisting of a monoclonal antibody produced by the hybridoma cell line deposited to the ATCC under Patent Deposit No.: PTA-4240 and a monoclonal antibody produced by the hybridoma cell line deposited to the ATCC under Patent Deposit No.: PTA-4241.


SEQUENCE LISTING (9 sequences)

SEQ ID NO: 1 - DNA, 2005 bp, Homo sapiens
atgcacggctcctgcagtttcctgatgcttctgctgccgctactgctactgctggtggccaccacaggccccgttggagccctcacagatgaggagaaacgtttgatggtggagctgcacaacctctaccgggcccaggtatccccgacggcctcagacatgctgcacatgagatgggacgaggagctggccgccttcgccaaggcctacgcacggcagtgcgtgtggggccacaacaaggagcgcgggcgccgcggcgagaatctgttcgccatcacagacgagggcatggacgtgccgctggccatggaggagtggcaccacgagcgtgagcactacaacctcagcgccgccacctgcagcccaggccagatgtgcggccactacacgcaggtggtatgggccaagacagagaggatcggctgtggttcccacttctgtgagaagctccagggtgttgaggagaccaacatcgaattactggtgtgcaactatgagcctccggggaacgtgaaggggaaacggccctaccaggaggggactccgtgctcccaatgtccctctggctaccactgcaagaactccctctgtggtgagtccacgggtggatggccccccacgcgcagccactttggcgccctgtcgttccaagtggccggatttcaacccttcaaagggaggatgttagaaagtctggcggcttcgggggggcccgcgcgagaacccatcggaagcccggaagatgctcaggatttgccttacctggtaactgaggccccatccttccgggcgactgaagcatcagactctaggaaaatgggtactccttcttccctagcaacggggattccggctttcttggtaacagaggtctcaggctccctggcaaccaaggctctgcctgctgtggaaacccaggccccaacttccttagcaacgaaagacccgccctccatggcaacagaggctccaccttgcgtaacaactgaggtcccttccattttggcagctcacagcctgccctccttggatgaggagccagttaccttccccaaatcgacccatgttcctatcccaaaatcagcagacaaagtgacagacaaaacaaaagtgccctctaggagcccagagaactctctggaccccaagatgtccctgacaggggcaagggaactcctaccccatgcccaggaggaggctgaggctgaggctgagttgcctccttccagtgaggtcttggcctcagtttttccagcccaggacaagccaggtgagctgcaggccacactggaccacacggggcacacctcctccaagtccctgcccaatttccccaatacctctgccaccgctaatgccacgggtgggcgtgccctggctctgcagtcgtccttgccaggtgcagagggccctgacaagcctagcgtcgtgtcagggctgaactcgggccctggtcatgtgtggggccctctcctgggactactgctcctgcctcctctggtgttggctggaatcttctgaaggggataccactcaaagggtgaagaggtcagctgtcctcctgtcatcttccccaccctgtccccagcccctaaacaagatacttcttggttaaggccctccggaagggaaaggctacggggcatgtgcctcatcacaccatccatcctggaggcacaaggcctggctggctgcgagctcaggaggccgcctgaggactgcacaccgggcccacacctctcctgcccctccctcctgagtcctgggggtgggaggatttgagggagctcactgcctacctggcctggggctgtctgcccacacagcatgtgcgctctccctgagtgcctgtgtagctggggatggggattcctaggggcagatgaaggacaagccccactggagtggggttctttgagtgggggaggcagggacgagggaaggaaagtaactcctgactctccaataaaaacctgtccaacctgtggcaaaaaaaaaaaaaaaa

SEQ ID NO: 2 - protein, 506 aa, Homo sapiens
MHGSCSFLMLLLPLLLLLVATTGPVGALTDEEKRLMVELHNLYRAQVSPTASDMLHMRWDEELAAFAKAYARQCVWGHNKERGRRGENLFAITDEGMDVPLAMEEWHHEREHYNLSAATCSPGQMCGHYTQVVWAKTERIGCGSHFCEKLQGVEETNIELLVCNYEPPGNVKGKRPYQEGTPCSQCPSGYHCKNSLCGESTGGWPPTRSHFGALSFQVAGFQPFKGRMLESLAASGGPAREPIGSPEDAQDLPYLVTEAPSFRATEASDSRKMGTPSSLATGIPAFLVTEVSGSLATKALPAVETQAPTSLATKDPPSMATEAPPCVTTEVPSILAAHSLPSLDEEPVTFPKSTHVPIPKSADKVTDKTKVPSRSPENSLDPKMSLTGARELLPHAQEEAEAEAELPPSSEVLASVFPAQDKPGELQATLDHTGHTSSKSLPNFPNTSATANATGGRALALQSSLPGAEGPDKPSVVSGLNSGPGHVWGPLLGLLLLPPLVLAGIF

SEQ ID NO: 3 - protein, 593 aa, Homo sapiens
  MISC_FEATURE (507)..(507): Xaa may be any amino acid  (e.g., Ala, Cys, Asp, Glu, Phe, Gly, His, Ile, Lys, Leu, Met, Asn, Pro, Gln, Arg, Ser, Thr, Val, Trp, Tyr)
MHGSCSFLMLLLPLLLLLVATTGPVGALTDEEKRLMVELHNLYRAQVSPTASDMLHMRWDEELAAFAKAYARQCVWGHNKERGRRGENLFAITDEGMDVPLAMEEWHHEREHYNLSAATCSPGQMCGHYTQVVWAKTERIGCGSHFCEKLQGVEETNIELLVCNYEPPGNVKGKRPYQEGTPCSQCPSGYHCKNSLCGESTGGWPPTRSHFGALSFQVAGFQPFKGRMLESLAASGGPAREPIGSPEDAQDLPYLVTEAPSFRATEASDSRKMGTPSSLATGIPAFLVTEVSGSLATKALPAVETQAPTSLATKDPPSMATEAPPCVTTEVPSILAAHSLPSLDEEPVTFPKSTHVPIPKSADKVTDKTKVPSRSPENSLDPKMSLTGARELLPHAQEEAEAEAELPPSSEVLASVFPAQDKPGELQATLDHTGHTSSKSLPNFPNTSATANATGGRALALQSSLPGAEGPDKPSVVSGLNSGPGHVWGPLLGLLLLPPLVLAGIFXRGYHSKGEEVSCPPVIFPTLSPAPKQDTSWLRPSGRERLRGMCLITPSILEAQGLAGCELRRPPEDCTPGPHLSCPSLLSPGGGRI

SEQ ID NO: 4 - DNA, 30 bp, Homo sapiens
atgcacggctcctgcagtttcctgatgctt

SEQ ID NO: 5 - DNA, 37 bp, Homo sapiens
gcccacgcgtcgactagtacttttttttttttttttt

SEQ ID NO: 6 - DNA, 1876 bp, Homo sapiens
atgcacggctcctgcagtttcctgatgcttctgctgccgctactgctactgctggtggccaccacaggccccgttggagccctcacagatgaggagaaacgtttgatggtggagctgcacaacctctaccgggcccaggtatccccgccggcctcagacatgctgcacatgagatgggacgaggagctggccgccttcgccaaggcctacgcacggcagtgcgtgtggggccacaacaaggagcgcgggcgccgcggcgagaatctgttcgccatcacagacgagggcatggacgtgccgctggccatggaggagtggcaccacgagcgtgagcactacaacctcagcgccgccacctgcagcccaggccagatgtgcggccactacacgcaggtggtatgggccaagacagagaggatcggctgtggttcccacttctgtgagaagctccagggtgttgaggagaccaacatcgaattactggtgtgcaactatgagcctccggggaacgtgaaggggaaacggccctaccaggaggggactccgtgctcccaatgtccctctggctaccactgcaagaactccctctgtgaacccatcggaagcccggaagatgctcaggatttgccttacctggtaactgaggccccatccttccgggcgactgaagcatcagactctaggaaaatgggtgctccttcttccctagcaacggggattccggctttcctggtcacaggggtgtcaggctcgctgccaaccctgggactgcctgctgtggaaacccaggccccaacttccttagcaacgaaagacccgccctccatggcaacagaggctccaccttgcgtaacaactgaggtcccttccattttggcagctcacagcctgccctccttggatgaggagccagttaccttccccaaatcgacccatgttcctatcccaaaatcagcagacaaagtgacagacaaaacaaaagtgccctctaggagcccagagaactctctggaccccaagatgtccctgacaggggcaagggaactcctaccccatgcccaggaggaggctgaggctgaggctgagttgcctccttccagtgaggtcttggcctcagtttttccagcccaggacaagccaggtgagctgcaggccacactggaccacacggggcacacctcctccaagtccctgcccaatttccccaatacctctgccaccgctaatgccacgggtgggcgtgccctggctctgcagtcgtccttgccaggtgcagagggccctgacaagcctagcgtcgtgtcagggctgaactcgggccctggtcatgtgtggggccctctcctgggactactgctcctgcctcctctggtgttggctggaatcttctgaaggggataccactcaaagggtgaagaggtcagctgtcctcctgtcatcttccccaccctgtccccagcccctaaacaagatacttcttggttaaggccctccggaagggaaaggctacggggcatgtgcctcatcacaccatccatcctggaggcacaaggcctggctggctgcgagctcaggaggccgcctgaggactgcacaccgggcccacacctctcctgcccctccctcctgagtcctgggggtgggaggatttgagggagctcactgcctacctggcctggggctgtctgcccacacagcatgtgcgctctccctgagtgcctgtgtagctggggatggggattcctaggggcagatgaaggacaagccccactggagtggggttctttgagtgggggaggcagggacgagggaaggaaagtaactcctgactctccaataaaaacctgtccaacctgtggcaaaaaaaaaaaaaaaa

SEQ ID NO: 7 - protein, 625 aa, Homo sapiens
  MISC_FEATURE (464)..(464): Xaa may be any amino acid (e.g., Ala, Cys, Asp, Glu, Phe, Gly, His, Ile, Lys, Leu, Met, Asn, Pro, Gln, Arg, Ser, Thr, Val, Trp, Tyr)
MHGSCSFLMLLLPLLLLLVATTGPVGALTDEEKRLMVELHNLYRAQVSPPASDMLHMRWDEELAAFAKAYARQCVWGHNKERGRRGENLFAITDEGMDVPLAMEEWHHEREHYNLSAATCSPGQMCGHYTQVVWAKTERIGCGSHFCEKLQGVEETNIELLVCNYEPPGNVKGKRPYQEGTPCSQCPSGYHCKNSLCEPIGSPEDAQDLPYLVTEAPSFRATEASDSRKMGAPSSLATGIPAFLVTGVSGSLPTLGLPAVETQAPTSLATKDPPSMATEAPPCVTTEVPSILAAHSLPSLDEEPVTFPKSTHVPIPKSADKVTDKTKVPSRSPENSLDPKMSLTGARELLPHAQEEAEAEAELPPSSEVLASVFPAQDKPGELQATLDHTGHTSSKSLPNFPNTSATANATGGRALALQSSLPGAEGPDKPSVVSGLNSGPGHVWGPLLGLLLLPPLVLAGIFXRGYHSKGEEVSCPPVIFPTLSPAPKQDTSWLRPSGRERLRGMCLITPSILEAQGLAGCELRRPPEDCTPGPHLSCPSLLSPGGGRIXGSSLPTWPGAVCPHSMCALPECLCSWGWGFLGADEGQAPLEWGSLSGGGRDEGRKVTPDSPIKTCPTCGKKKKK

SEQ ID NO: 8 - protein, 550 aa, Homo sapiens
  MISC_FEATURE (464)..(464): Xaa may be any amino acid (e.g., Ala, Cys, Asp, Glu, Phe, Gly, His, Ile, Lys, Leu, Met, Asn, Pro, Gln, Arg, Ser, Thr, Val, Trp, Tyr)
MHGSCSFLMLLLPLLLLLVATTGPVGALTDEEKRLMVELHNLYRAQVSPPASDMLHMRWDEELAAFAKAYARQCVWGHNKERGRRGENLFAITDEGMDVPLAMEEWHHEREHYNLSAATCSPGQMCGHYTQVVWAKTERIGCGSHFCEKLQGVEETNIELLVCNYEPPGNVKGKRPYQEGTPCSQCPSGYHCKNSLCEPIGSPEDAQDLPYLVTEAPSFRATEASDSRKMGAPSSLATGIPAFLVTGVSGSLPTLGLPAVETQAPTSLATKDPPSMATEAPPCVTTEVPSILAAHSLPSLDEEPVTFPKSTHVPIPKSADKVTDKTKVPSRSPENSLDPKMSLTGARELLPHAQEEAEAEAELPPSSEVLASVFPAQDKPGELQATLDHTGHTSSKSLPNFPNTSATANATGGRALALQSSLPGAEGPDKPSVVSGLNSGPGHVWGPLLGLLLLPPLVLAGIFXRGYHSKGEEVSCPPVIFPTLSPAPKQDTSWLRPSGRERLRGMCLITPSILEAQGLAGCELRRPPEDCTPGPHLSCPSLLSPGGGRI

SEQ ID NO: 9 - protein, 463 aa, Homo sapiens
MHGSCSFLMLLLPLLLLLVATTGPVGALTDEEKRLMVELHNLYRAQVSPPASDMLHMRWDEELAAFAKAYARQCVWGHNKERGRRGENLFAITDEGMDVPLAMEEWHHEREHYNLSAATCSPGQMCGHYTQVVWAKTERIGCGSHFCEKLQGVEETNIELLVCNYEPPGNVKGKRPYQEGTPCSQCPSGYHCKNSLCEPIGSPEDAQDLPYLVTEAPSFRATEASDSRKMGAPSSLATGIPAFLVTGVSGSLPTLGLPAVETQAPTSLATKDPPSMATEAPPCVTTEVPSILAAHSLPSLDEEPVTFPKSTHVPIPKSADKVTDKTKVPSRSPENSLDPKMSLTGARELLPHAQEEAEAEAELPPSSEVLASVFPAQDKPGELQATLDHTGHTSSKSLPNFPNTSATANATGGRALALQSSLPGAEGPDKPSVVSGLNSGPGHVWGPLLGLLLLPPLVLAGIF